(19) United States
(12) Patent Application Publication
(10) Pub. No.: US 2026/0048050 A1
(43) Pub. Date: Feb. 19, 2026

(54) PHARMACEUTICAL COMPOSITIONS AND METHODS OF TREATING ENTEROVIRUSES WITH VAPENDAVIR SULFATE

(71) Applicant: ALTESA BIOSCIENCES, INC., College Park, GA (US)

(72) Inventors: John Michael CLERICI, Reston, VA (US); Brett Paul GIROIR, College Station, TX (US); Melissa ALLABAND, Holly Springs, NC (US); Jeffrey Lee BRUM, Pottstown, PA (US); Katherine LAESSIG, Washington, DC (US); Alan Frank PARR, Acworth, GA (US); Paul Grant SPOORS, Downingtown, PA (US); Katherine Elizabeth SQUIRES, Atlanta, GA (US)

(73) Assignee: ALTESA BIOSCIENCES, INC., College Park, GA (US)

(21) Appl. No.: 19/304,285

(22) Filed: Aug. 19, 2025

Related U.S. Application Data
(60) Provisional application No. 63/684,652, filed on Aug. 19, 2024.
(60) Provisional application No. 63/684,655, filed on Aug. 19, 2024.
(60) Provisional application No. 63/684,661, filed on Aug. 19, 2024.
(60) Provisional application No. 63/694,305, filed on Sep. 13, 2024.
(60) Provisional application No. 63/715,172, filed on Nov. 1, 2024.
(60) Provisional application No. 63/715,272, filed on Nov. 1, 2024.
(60) Provisional application No. 63/715,299, filed on Nov. 1, 2024.
(60) Provisional application No. 63/715,323, filed on Nov. 1, 2024.
(60) Provisional application No. 63/743,420, filed on Jan. 9, 2025.
(60) Provisional application No. 63/804,845, filed on May 13, 2025.
(60) Provisional application No. 63/814,168, filed on May 29, 2025.

(51) Int. Cl. A61K 31/501; A61P 31/16

(52) U.S. Cl. CPC A61K 31/501; A61P 31/16

(57) ABSTRACT

The present disclosure describes a sulfate salt form of vapendavir and a pharmaceutical composition containing the same. Methods of treating a subject having a respiratory enterovirus by administering to the subject a pharmaceutical composition including a therapeutically effective amount of the vapendavir sulfate are also described, wherein the subject may or may not also have COPD or asthma.

### CROSS-REFERENCE TO RELATED APPLICATIONS

This application claims priority to U.S. Provisional Patent Application Nos. 63/684,652 filed on Aug. 19, 2024, 63/715,272 filed on Nov. 1, 2024, 63/804,845 filed on May 13, 2025, 63/715,172 filed on Nov. 1, 2024, 63/684,655 filed on Aug. 19, 2024, 63/715,299 filed on Nov. 1, 2024, 63/694,305 filed on Sep. 13, 2024, 63/684,661 filed on Aug. 19, 2024, 63/814,168 filed on May 29, 2025, 63/715,323 filed on Nov. 1, 2024, and 63/743,420 filed on Jan. 9, 2025, which are hereby incorporated by reference herein in their entirety.

### BACKGROUND

Rhinoviruses (RVs) are small RNA viruses that represent significant diversity within the Enterovirus (EV) genus, with over 180 distinct strains. RVs are the most common cause of upper respiratory tract infections (UTRI) and a frequent cause of community-acquired pneumonia in adults. While RVs are normally associated with mild and self-limiting cold-like symptoms, RVs can cause significant morbidity and mortality via the exacerbation of chronic respiratory diseases such as asthma, cystic fibrosis, primary ciliary dyskinesia, or COPD.

Acute exacerbations are the primary contributors to the morbidity and mortality associated with COPD. These exacerbations are associated with an impaired quality of life and accelerated deterioration of lung function. Approximately 40-50% of COPD exacerbations are linked to RV infection. However, current therapies for managing COPD and treating acute exacerbations are aimed at palliating the symptoms and, in many cases, have significant side effects. Current therapies such as long-acting muscarinic receptor antagonists (LAMAs), long acting β₂ receptor agonists (LABAs), inhaled corticosteroids (ICS), and antibacterial drugs are only partially effective and fail to address the underlying mechanisms of disease or triggers of exacerbation.

Vapendavir (VPV) is a potent, broad spectrum antiviral drug active against a majority of RV serotypes and other enteroviruses evaluated in cell-based assay systems and human clinical trials. Vapendavir has been tested in humans with no appreciable safety concerns noted following single and multiple doses. There remains a need for an effective treatment for RV infection, particularly in subjects having chronic respiratory conditions such as COPD.

### SUMMARY

In some aspects, the techniques described herein relate to a pharmaceutical composition including a therapeutically effective amount of vapendavir sulfate and one or more pharmaceutically acceptable excipients, wherein the vapendavir sulfate is in the form of Crystalline Form A, wherein the vapendavir sulfate has a particle size distribution with a D90 of about 35 μm to about 140 μm, a D50 of about 10 μm to about 25 μm, and a D10 of about 2.5 μm to about 10 μm, and wherein the pharmaceutical composition is a solid oral dosage form.

In some aspects, the techniques described herein relate to a pharmaceutical composition, wherein the therapeutically effective amount of vapendavir sulfate is about 100 mg to about 1000 mg.

In some aspects, the techniques described herein relate to a pharmaceutical composition, wherein the therapeutically effective amount of vapendavir sulfate is about 250 mg to about 800 mg.

In some aspects, the techniques described herein relate to a pharmaceutical composition, wherein the therapeutically effective amount of vapendavir sulfate is about 500 mg.

In some aspects, the techniques described herein relate to a pharmaceutical composition, wherein the vapendavir sulfate has a polymorphic purity of at least about 90%.

In some aspects, the techniques described herein relate to a pharmaceutical composition, wherein the vapendavir sulfate has a particle size distribution with a D90 of about 100 μm, a D50 of about 25 μm, and a D10 of about 10 μm.

In some aspects, the techniques described herein relate to a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days, wherein orally administering the loading dose to the subject results in a Cmax of about 2400 ng/mL to about 3750 ng/mL of vapendavir; and wherein the subject is in a fasted state when orally administering the loading dose and the maintenance doses; wherein the respiratory enterovirus in the subject is treated.

In some aspects, the techniques described herein relate to a method, wherein orally administering the loading dose to the subject results in a Cmax of about 3000 ng/mL.

In some aspects, the techniques described herein relate to a method, wherein the subject has COPD.

In some aspects, the techniques described herein relate to a method, wherein the subject has asthma.

In some aspects, the techniques described herein relate to a method, wherein a Tmax is about 2 hours.

In some aspects, the techniques described herein relate to a method, wherein the subject is not taking an acid reducer when orally administering the loading dose and the maintenance doses.

In some aspects, the techniques described herein relate to a method, wherein the loading dose is administered within about 1 to about 7 days of the subject displaying symptoms of the respiratory enterovirus.

In some aspects, the techniques described herein relate to a method, wherein treating the respiratory enterovirus in the subject includes decreasing a viral load in the sputum, nasal passages, or a combination thereof in the subject compared with a viral load in the sputum, nasal passages, or a combination thereof of the subject prior to treatment.

In some aspects, the techniques described herein relate to a method, wherein treating the respiratory enterovirus in the subject includes reducing the duration of viral shedding.

In some aspects, the techniques described herein relate to a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days, wherein orally administering the loading dose to the subject results in a Cmax of about 6400 ng/mL to about 10000 ng/mL of vapendavir; and wherein the subject is in a fed state when orally administering the loading dose and the maintenance doses; wherein the respiratory enterovirus in the subject is treated.

In some aspects, the techniques described herein relate to a method, wherein orally administering the loading dose to the subject results in a Cmax of about 8000 ng/mL.

In some aspects, the techniques described herein relate to a method, wherein the subject has COPD.

In some aspects, the techniques described herein relate to a method, wherein the subject has asthma.

In some aspects, the techniques described herein relate to a method, wherein a Tmax is about 4 hours.

In some aspects, the techniques described herein relate to a method, wherein the subject is not taking an acid reducer when orally administering the loading dose and the maintenance doses.

In some aspects, the techniques described herein relate to a method, wherein the loading dose is administered within about 1 to about 7 days of the subject displaying symptoms of the respiratory enterovirus.

In some aspects, the techniques described herein relate to a method, wherein treating the respiratory enterovirus in the subject includes decreasing a viral load in the sputum, nasal passages, or a combination thereof in the subject compared with a viral load in the sputum, nasal passages, or a combination thereof of the subject prior to treatment.

In some aspects, the techniques described herein relate to a method, wherein treating the respiratory enterovirus in the subject includes reducing the duration of viral shedding.

In some aspects, the techniques described herein relate to a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days, wherein orally administering the loading dose to the subject results in a Cmax of about 2400 ng/mL to about 3750 ng/mL of vapendavir; wherein the subject is in a fed state when orally administering the loading dose and the maintenance doses; and wherein the subject is taking an acid reducer when orally administering the loading dose and the maintenance doses; wherein the respiratory enterovirus in the subject is treated.

In some aspects, the techniques described herein relate to a method, wherein orally administering the loading dose to the subject results in a Cmax of about 3000 ng/mL.

In some aspects, the techniques described herein relate to a method, wherein the subject has COPD.

In some aspects, the techniques described herein relate to a method, wherein the subject has asthma.

In some aspects, the techniques described herein relate to a method, wherein a Tmax is about 6 hours.

In some aspects, the techniques described herein relate to a method, wherein the loading dose is administered within about 1 to about 7 days of the subject displaying symptoms of the respiratory enterovirus.

In some aspects, the techniques described herein relate to a method, wherein treating the respiratory enterovirus in the subject includes decreasing a viral load in the sputum, nasal passages, or a combination thereof in the subject compared with a viral load in the sputum, nasal passages, or a combination thereof of the subject prior to treatment.

In some aspects, the techniques described herein relate to a method, wherein treating the respiratory enterovirus in the subject includes reducing the duration of viral shedding.

In some aspects, the techniques described herein relate to a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days, wherein orally administering the loading dose to the subject results in a Cmax of about 1600 ng/mL to about 2500 ng/mL of vapendavir; wherein the subject is in a fasted state when orally administering the loading dose and the maintenance doses; and wherein the subject is taking an acid reducer when orally administering the loading dose and the maintenance doses; wherein the respiratory enterovirus in the subject is treated.

In some aspects, the techniques described herein relate to a method, wherein orally administering the loading dose to the subject results in a Cmax of about 2000 ng/mL.

In some aspects, the techniques described herein relate to a method, wherein the subject has COPD.

In some aspects, the techniques described herein relate to a method, wherein the subject has asthma.

In some aspects, the techniques described herein relate to a method, wherein a Tmax is about 2 hours.

In some aspects, the techniques described herein relate to a method, wherein the loading dose is administered within about 1 to about 7 days of the subject displaying symptoms of the respiratory enterovirus.

In some aspects, the techniques described herein relate to a method, wherein treating the respiratory enterovirus in the subject includes decreasing a viral load in the sputum, nasal passages, or a combination thereof in the subject compared with a viral load in the sputum, nasal passages, or a combination thereof of the subject prior to treatment.

In some aspects, the techniques described herein relate to a method, wherein treating the respiratory enterovirus in the subject includes reducing the duration of viral shedding.

In some aspects, the techniques described herein relate to a method of treating a respiratory enterovirus in a subject in need thereof, including orally administering to the subject a loading dose of vapendavir sulfate in an amount sufficient to provide about 1000 mg of vapendavir free base, followed by orally administering a maintenance dose of vapendavir sulfate in an amount sufficient to provide about 1000 mg of vapendavir free base daily for about 5 days to about 10 days, wherein the subject is in a fed state when administering the loading dose and the maintenance doses, and wherein the respiratory enterovirus in the subject is treated.

In some aspects, the techniques described herein relate to a method, wherein orally administering the loading dose to the subject results in a Cmax of about 6400 ng/mL to about 10000 ng/mL of vapendavir.

In some aspects, the techniques described herein relate to a method, wherein orally administering the loading dose to the subject results in a Cmax of about 8000 ng/mL of vapendavir.

In some aspects, the techniques described herein relate to a method, wherein a Tmax is about 4 hours.

In some aspects, the techniques described herein relate to a method, wherein the subject has COPD.

In some aspects, the techniques described herein relate to a method, wherein the subject has asthma.

In some aspects, the techniques described herein relate to a method, wherein the subject is not taking an acid reducer when orally administering the loading dose and the maintenance doses.

In some aspects, the techniques described herein relate to a method, wherein the loading dose is first administered within about 1 to about 7 days of the subject displaying symptoms of the respiratory enterovirus.

In some aspects, the techniques described herein relate to a method, wherein treating the respiratory enterovirus in the subject includes decreasing a viral load in the sputum, nasal passages, or a combination thereof in the subject compared with a viral load in the sputum, nasal passages, or a combination thereof of the subject prior to treatment.

In some aspects, the techniques described herein relate to a method, wherein treating the respiratory enterovirus in the subject includes reducing the duration of viral shedding.

In some aspects, the techniques described herein relate to a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base daily for about 5 to about 10 days, wherein the subject is in a fed state when administering the loading dose and the maintenance doses, and wherein the respiratory enterovirus is treated.

In some aspects, the techniques described herein relate to a method, wherein orally administering the loading dose to the subject results in a Cmax of about 6400 ng/mL to about 10000 ng/mL of vapendavir.

In some aspects, the techniques described herein relate to a method, wherein orally administering the loading dose to the subject results in a Cmax of about 8000 ng/mL of vapendavir.

In some aspects, the techniques described herein relate to a method, wherein a Tmax is about 4 hours.

In some aspects, the techniques described herein relate to a method, wherein the subject has COPD.

In some aspects, the techniques described herein relate to a method, wherein the subject has asthma.

In some aspects, the techniques described herein relate to a method, wherein treating the respiratory enterovirus in the subject includes decreasing a viral load in the sputum, nasal passages, or a combination thereof in the subject compared with a viral load in the sputum, nasal passages, or a combination thereof of the subject prior to treatment.

In some aspects, the techniques described herein relate to a method, wherein treating the respiratory enterovirus in the subject includes reducing the duration of viral shedding.

In some aspects, the techniques described herein relate to a method, wherein the subject is not taking an acid reducer when orally administering the loading dose and the maintenance doses.

In some aspects, the techniques described herein relate to a method, wherein the loading dose is administered within about 1 to about 7 days of the subject displaying symptoms of the respiratory enterovirus.

In some aspects, the techniques described herein relate to a method, wherein treating the respiratory enterovirus in the subject includes decreasing a viral load in the sputum, nasal passages, or a combination thereof in the subject compared with a viral load in the sputum, nasal passages, or a combination thereof of the subject prior to treatment.

In some aspects, the techniques described herein relate to a method, wherein treating the respiratory enterovirus in the subject includes reducing the duration of viral shedding.

### DRAWINGS

Aspects, features, benefits, and advantages of the embodiments described herein will be apparent with regard to the following description, appended claims, and accompanying drawings where:

FIG. 1 is an XRPD diffractogram of Crystalline Form A of vapendavir bisulfate, according to embodiments the present disclosure.

FIG. 2A is a bar graph showing the equilibrium solubility of vapendavir free base in FaSSIF, FeSSIF, FaSSGF and solutions of various pH. FIG. 2B is a bar graph showing the equilibrium solubility of vapendavir bisulfate in FaSSIF, FeSSIF, FaSSGF, and solutions of various pH, according to an embodiment of the present disclosure.

FIG. 3 is a thermal ellipsoid rendering of the molecular structure of vapendavir bisulfate single crystal structure, displayed at 50% confidence interval, showing atomic numbering.

FIG. 4A is the packing diagram of vapendavir bisulfate viewed along the a-axis. FIG. 4B is the packing diagram of vapendavir bisulfate viewed along the b-axis. FIG. 4C is the packing diagram of vapendavir bisulfate viewed along the c-axis. The intermolecular interactions which influence the packing arrangement are also shown. FIG. 4D shows the hydrogen-bonding network in vapendavir bisulfate, viewed down the a-axis, with hydrogen atoms omitted for clarity. FIG. 4E shows hydrogen-bonding network in vapendavir bisulfate, viewed down the c-axis, with hydrogen atoms omitted for clarity

FIG. 5 is a graph of absorption of vapendavir bisulfate when co-administered with famotidine or pentagastrin, according to an embodiment of the present disclosure.

FIG. 6 is a graph comparing the solubility of vapendavir free base, vapendavir phosphate, and vapendavir bisulfate in FaSSGF (pH 1.61) and FaSSIF (pH 6.52) solutions, according to an embodiment of the present disclosure.

FIG. 7 is a graph of the solubility of vapendavir bisulfate as produced and micronized vapendavir bisulfate, according to embodiments of the present disclosure.

FIG. 8 is a graph of absorption of vapendavir bisulfate and vapendavir free base when co-administered with famotidine or pentagastrin, according to an embodiment of the present disclosure.

FIG. 9A shows the concentration of vapendavir bisulfate administered to a fasted subject, according to an embodiment of the present disclosure, compared to the concentration of vapendavir free base administered to a fasted subject. FIG. 9B shows the concentration of vapendavir bisulfate administered to a fasted subject and a fed subject, according to embodiments of the present disclosure, compared to the concentration of vapendavir free base administered to a fasted subject. FIG. 9C shows the concentration of vapendavir bisulfate administered to a fasted subject and a fed subject, according to embodiments of the present disclosure, compared to the concentration of vapendavir free base administered to a fasted subject, plotted on a logarithmic scale. FIG. 9D shows the concentration of vapendavir bisulfate administered to a fasted subject, a fed subject, and a fed subject taking a PPI, according to embodiments of the present disclosure.

FIG. 9E shows the concentration of vapendavir bisulfate administered to a fed subject and a fed subject taking a PPI, according to embodiments of the present disclosure, plotted on a logarithmic scale. FIG. 9F shows the concentration of vapendavir bisulfate administered to a fasted subject and a fasted subject taking a PPI, according to embodiments of the present disclosure, plotted on a logarithmic scale.

FIG. 10 is a diagram of the study design in a clinical study of vapendavir for the treatment of respiratory enterovirus infections in subjects with COPD.

FIG. 11 depicts mean vapendavir plasma concentrations vs time profiles (Left=Linear Scale, Right=Log Scale) for normal healthy volunteers (Group A) (Periods 1 and 2 (Fast/Fed).

FIG. 12 depicts mean vapendavir plasma concentrations vs time profiles (Left=Linear Scale, Right=Log Scale) normal healthy volunteers (Group A) (Treatment C) (Days 1-7). Day 1=VPV 1000 mg am and 500 mg pm; Day 4=VPV 500 mg BID; Day 7=VPV 500 mg BID.

FIG. 13 depicts mean vapendavir plasma concentrations vs time profiles (Left=Linear Scale, Right=Log Scale) for participants with a diagnosis of COPD (Group B)—Treatment C (Days 1-7). SD=Standard deviation Day 1=VPV 1000 mg am and 500 mg pm; Day 4=VPV 500 mg BID; Day 7=VPV 500 mg BID.

FIG. 14 depicts the mean plasma concentration following administration of VPV for Group B—Treatment C (Days 1-7 (Day 1=VPV 1000 mg am and 500 mg pm; Day 4=VPV 500 mg BID; Day 7=VPV 500 mg BID)) and shows the average protein adjusted EC₅₀ for human rhinoviruses.

FIG. 15 depicts the mean plasma concentration in participants with COPD dosed with a 1,000 mg loading dose followed by 500 mg BID for a full 7-day course.

FIG. 16 depicts the mean Cₘₐₓ in fasted versus fed participants with COPD after 1, 4 and 7 days for the fasted group (Group B—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course).

FIG. 17 depicts the mean Cₘₐₓ in fasted versus fed participants with COPD as well as normal healthy volunteers after 1, 4 and 7 days for the fasted group (Group A (normal healthy volunteers) and Group B (COPD)—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course).

FIG. 18 depicts the mean Cₘᵢₙ (12 hours) in fasted versus fed participants with COPD after 1, 4 and 7 days for the fasted group (Group B—Treatment C (Days 1-7 (Day 1=VPV 1000 mg am and 500 mg pm; Day 4=VPV 500 mg BID; Day 7=VPV 500 mg BID))) and 1, 2, and 7 days for the fed group (1000 mg loading dose followed by 500 mg BID for a full 7-day course).

FIG. 19 depicts the mean Cₘᵢₙ (12 hours) in fasted versus fed participants with COPD as well as normal healthy volunteers after 1, 4 and 7 days for the fasted group (Group A (normal healthy volunteers) and Group B (COPD)—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course).

FIG. 20 depicts Cₘₐₓ for each day of treatment for each of the COPD subjects in the study where each subject was fed.

FIG. 21 depicts Cₘₐₓ for each day of treatment for each of the COPD subjects in the study where each subject was fasted (Group B).

FIG. 22 depicts the coefficient of variation for the Cₘₐₓ in fasted versus fed participants with COPD after 1, 4 and 7 days for the fasted group (Group B—Treatment C (Days 1-7 (Day 1=VPV 1000 mg am and 500 mg pm; Day 4=VPV 500 mg BID; Day 7=VPV 500 mg BID))) and 1, 2, and 7 days for the fed group (1000 mg loading dose followed by 500 mg BID for a full 7-day course).

FIG. 23 depicts the coefficient of variation for the Cₘᵢₙ in fasted versus fed participants with COPD after 1, 4 and 7 days for the fasted group (Group B—Treatment C (Days 1-7 (Day 1=VPV 1000 mg am and 500 mg pm; Day 4=VPV 500 mg BID; Day 7=VPV 500 mg BID))) and 1, 2, and 7 days for the fed group (1000 mg loading dose followed by 500 mg BID for a full 7-day course).

FIG. 24 depicts Kₑₗ (Elimination Rate Constant) for each of the 6 Participants for the fed group (1000 mg loading dose followed by 500 mg BID for a full 7-day course) at day 7.

FIG. 25 shows the Cₘᵢₙ results of a PK study vs. challenge study in fed COPD subjects.

FIG. 26 shows challenge study Cₘᵢₙ results for each of the 11 VPV participants at day 1 and day 2.

FIG. 27 shows the Cₘᵢₙ coefficient of variation for the PK and challenge studies.

FIG. 28 shows LRSS—Total Score AUC from day of treatment commencement to day 21 for VPV subjects and placebo subjects.

FIG. 29 shows EXACT-RS—Total Score Peak from day of treatment commencement to day 21 for VPV subjects and placebo subjects.

FIG. 30 shows EXACT-RS—Total Score AUC from day of treatment commencement to day 21 for VPV subjects and placebo subjects.

FIG. 31 shows EXACT-RS—Number of days that EXACT-RS total score was greater than baseline from day of treatment commencement to day 21 for VPV subjects and placebo subjects.

FIG. 32 shows EXACT-RS—Time to resolution from day of treatment commencement to day 21 for VPV subjects and placebo subjects.

FIG. 33 shows EXACT-RS—Number of days with change in total EXACT-RS>=2 points from baseline from day of treatment commencement to day 21 for VPV subjects and placebo subjects.

FIG. 34 shows URSS—Total Score Peak from day of treatment commencement to day 21 for VPV subjects and placebo subjects.

FIG. 35 shows URSS—Total Score AUC from day of treatment commencement to day 21 for VPV subjects and placebo subjects.

FIG. 36 shows URSS—Time to resolution from day of treatment commencement to day 21 for VPV subjects and placebo subjects.

FIG. 37 shows the peak viral load in nasal lavage for VPV and placebo.

FIG. 38 shows the viral load in sputum for VPV and placebo.

FIG. 39 shows the peak viral load in sputum for VPV and placebo.

FIG. 40 shows the URSS AUC mean change from baseline (day of treatment commencement to day 21, at 95% confidence intervals) of placebo vs. vapendavir, according to an embodiment of the present disclosure.

FIG. 41 shows the LRSS AUC mean change from baseline (day of treatment commencement to day 21, at 95% confidence intervals) of placebo vs. vapendavir, according to an embodiment of the present disclosure.

FIG. 42 shows the E-RS AUC mean change from baseline (day of treatment commencement to day 21, at 95% confidence intervals) of placebo vs. vapendavir, according to an embodiment of the present disclosure.

FIG. 43 shows the nasal swab viral load over time with placebo (n=8) vs. vapendavir (n=5), according to an embodiment of the present disclosure. As shown, vapendavir lowered viral load quickly after treatment.

FIG. 44 is a scatter plot of nasal swab viral load over time with placebo (n=8) vs. vapendavir (n=5), according to an embodiment of the present disclosure.

FIG. 45 is a bar graph of AUC virus load (log(copies/mL)*days) of placebo (n=8) compared to vapendavir (n=5), according to an embodiment of the present disclosure.

FIG. 46A and FIG. 46B show % virus as a function of vapendavir free base concentration in cells from asthmatic donors, according to an embodiment of the present disclosure.

### DETAILED DESCRIPTION

Before compounds, compositions and methods are described in detail, it is to be understood that this disclosure is not limited to the particular processes, compositions, or methodologies described, as these may vary. It is also to be understood that the terminology used in the description is for the purpose of describing the particular versions or embodiments only, and is not intended to limit the scope of the disclosure which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the disclosure, the preferred methods, devices, and materials are now described.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein, the term “about” means plus or minus up to 10% of the numerical value of the number with which it is being used. For example, “about 50%” means in the range of 45-55% and includes exactly 50%. The term “about” may refer to plus or minus 1%, 5%, or 10% of the numerical value of the number with which it is being used.

“Administering,” when used in conjunction with the compounds of the disclosure, means to administer a compound directly into or onto a target tissue or to administer a compound systemically or locally to a subject or other subject.

As used herein, the term “free base” refers to a non-salt form of a compound as described herein.

The term “salts” refers to the common meaning in the art. The salts, as described herein, may be pharmaceutically acceptable. Examples of acids which may be employed to form pharmaceutically acceptable acid addition salts include but are not limited to salts derived from nontoxic inorganic acids such as nitric, phosphoric, sulfuric, hydrobromic, hydroiodic, hydrofluoric, or phosphorus acids, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, hydrochloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Salts of amino acids are also contemplated, such as arginate, gluconate, galacturonate, and the like.

Those skilled in the art of organic chemistry will appreciate that many organic compounds can form complexes with solvents in which they are reacted or from which they are precipitated or crystallized. For example, a complex with water is known as a “hydrate.” Solvates of the compounds of the present disclosure are within the scope of this disclosure. The salts of the compound of any of the formulae described herein may form solvates (e.g., hydrates) and the present disclosure includes all such solvates. The meaning of the word “solvates” is well known to those skilled in the art as a compound formed by interaction of a solvent and a solute (i.e., solvation). Techniques for the preparation of solvates are well established in the art.

Reference is made herein to DXX values, such as D10, D50, and D90, to describe particle size distribution. As used herein, a population of particles having a D90 value of x μm means that 90% of the particles are equal to or smaller than x μm. For example, a D90 of 60 μm means that 90% of particles are equal to or smaller than 60 μm. Similarly, a D50 value of x μm means that 50% of the particles are equal to or smaller than x μm, and a D10 value of x μm means that 10% of the particles are equal to or smaller than x μm. It is contemplated that other units may be used in DXX values as appropriate, such as nm, mm, and so forth.

The phrase “pharmaceutically acceptable” refers to molecular entities and compositions that are generally regarded as safe and non-toxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a subject. In some embodiments, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The phrase “pharmaceutically acceptable salt(s)”, as used herein, includes those salts of compounds of the disclosure that are safe and effective for use in mammals and that possess the desired biological activity. Pharmaceutically acceptable salts include salts of acidic or basic groups present in compounds of the disclosure or in compounds identified pursuant to the methods of the disclosure.

As used herein, the term “therapeutic” means an agent utilized to treat, combat, ameliorate, protect against, or improve an unwanted condition or disease of a subject.

A “therapeutically effective amount” or “effective amount” of a compound, such as vapendavir, or composition of the disclosure is a predetermined amount which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change). The effect contemplated herein includes medical therapeutic treatment, as appropriate. The specific dose of a compound administered according to this disclosure to obtain therapeutic effects will, of course, be determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the subject; the time of administration, route of administration, and rate of excretion of the specific compound employed and the duration of the treatment. The effective amount administered will be determined by the physician in the light of the foregoing relevant circumstances and the exercise of sound medical judgment. A therapeutically effective amount of a compound of this disclosure, such as vapendavir, is typically an amount such that when it is administered in a physiologically tolerable excipient composition, it is sufficient to achieve an effective systemic concentration or local concentration in the tissue.

The terms “treat”, “treated”, or “treating” as used herein refers to therapeutic treatment measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

As used herein, the term “adverse event” refers to any untoward medical occurrence associated with the use of a drug in humans, whether or not considered drug related. The abbreviation “AE” may be used to refer to an adverse event.

As used herein, the abbreviation “ADL” means activities of daily living.

As used herein, the abbreviation “ALT” means alanine aminotransferase.

As used herein, the abbreviation “AUC” means area under the curve.

As used herein, the abbreviation “BID” means bis in de and refers to twice a day dosage.

As used herein, the abbreviation “BL” means baseline.

As used herein, the abbreviation “CF” means cystic fibrosis.

As used herein, the abbreviation “Cₘₐₓ” means peak drug concentration.

As used herein, the abbreviation “IPF” means Idiopathic Pulmonary Fibrosis.

As used herein, the abbreviation “CYP3A2” means Cytochrome P450 family 1 subfamily A member 2.

As used herein, the abbreviation “CYP3A4” means Cytochrome P450 family 3 subfamily A member 4.

As used herein, the abbreviation “CXCL8” means C—X—C motif chemokine ligand 8.

As used herein, the abbreviation “CXCL10” means C—X—C motif chemokine ligand 10.

As used herein, “EC₅₀” refers to half maximal effective concentration.

As used herein, “EC₉₀” refers to 90% effective concentration.

As used herein, “paEC₅₀” refers to the protein adjusted half maximal effective concentration.

As used herein, “paEC₉₀” refers to the protein adjusted 90% effective concentration.

As used herein, “FEV₁” refers to Forced Expiratory Volume in 1 second.

As used herein, “FVC” means Forced Vital Capacity.

Unless otherwise indicated, the abbreviation “h” refers to hours as a unit of time.

As used herein, the abbreviation “Scr” means screening.

As used herein, the abbreviation “IC” means informed consent.

As used herein, the abbreviation “NM” means medical history.

As used herein, the abbreviation “IE” means inclusion/exclusion criteria.

As used herein, the abbreviation “IC₅₀” refers to half maximal inhibitory concentration.

As used herein, the abbreviation “PT” means pregnancy test.

As used herein, the abbreviation “T” means training of diaries, unless otherwise indicated.

As used herein, the abbreviation “D” means diaries.

As used herein, the abbreviation “PE” means physical examination.

As used herein, the abbreviation “PEF” means peak expiratory flow.

As used herein, the abbreviation “PK” means pharmacokinetics.

As used herein, the abbreviation “LF” means lung function.

As used herein, the abbreviation “NL” means nasal lavage and nasosorption.

As used herein, the abbreviation “Bl” means blood chemistry and full blood count.

As used herein, the abbreviation “Bl-Ser” means RV-A16 serology, mediators.

As used herein, the abbreviation “Bl-PK” means pharmacodynamic testing of blood.

As used herein, the abbreviation “SAE” means serious adverse event.

As used herein, the abbreviation “Sp” means sputum.

As used herein, the abbreviation “SM” means safety assessment.

As used herein, the abbreviation “CM” means concomitant medications.

Reference is made herein to “polymorphs” and “crystalline forms.” It will be understood by those skilled in the art that compounds in a crystalline form can exhibit polymorphism, and multiple polymorphs may be observed for a single compound, either in a free base or salt form. It will be further understood that while the term “polymorph” suggests multiple crystalline forms, it is common in the art to refer to a crystalline form as “a polymorph” regardless of how many distinct crystalline forms have been observed. The free base and salt forms of a compound may each exhibit polymorphism, or polymorphism may only be observed in one of the free base or salt form. The polymorphs of a free base or a salt may exhibit distinct properties and may be distinguishable by characterization methods including but not limited to X-ray powder diffraction (XRPD) and Raman spectroscopy. Different polymorphs of a compound or salt may exhibit distinct melting points, solubility, physical characteristics, and biological or therapeutic activity, among other factors. It will be further understood by those skilled in the art that hydrates or solvates of a free base or salt can also exhibit polymorphism, and may have distinct properties from the anhydrous or non-solvated free base or salt.

As used herein, “vapendavir sulfate” or “the sulfate salt of vapendavir” may refer to sulfate salts of vapendavir, including vapendavir bisulfate (mono-protonated, HSO₄⁻ bisulfate counterion), vapendavir sulfate (bis-protonated, SO₄²⁻ sulfate counterion), or combinations thereof. The “vapendavir sulfate” or “the sulfate salt of vapendavir” described herein may include vapendavir bisulfate only, vapendavir sulfate only, or any ratio of vapendavir bisulfate and vapendavir sulfate. Use of the term “vapendavir bisulfate” is not intended to exclude the presence of bis-protonated vapendavir sulfate. While embodiments may be presented as “vapendavir sulfate” or “the sulfate salt of vapendavir”, each embodiment is also intended to be presented as vapendavir bisulfate (mono-protonated, HSO₄⁻ bisulfate counterion), vapendavir sulfate (bis-protonated, SO₄²⁻ sulfate counterion), or any combination thereof, despite the fact that such embodiment is not specifically repeated herein. When a specific sulfate salt is described in an example, the example is intended to refer to the specific sulfate salt described therein.

COPD is the third leading cause of death worldwide and fourth leading cause in the United States (US). Among adults with COPD in the US, there are approximately 1.5 million emergency department (ED) visits, 700,000 hospitalizations, and over 10 million office visits annually. According to the Center for Disease Control and Prevention (CDC), costs associated with COPD in the US were approximately $49 billion in 2020, increasing 52.6% from 2010.

Acute exacerbations resulting from rhinovirus (RV) and other enterovirus (EV) infections, are the primary contributors to the morbidity and mortality associated with COPD. Approximately 40 to 50% of COPD exacerbations (range 10 to 70%) are linked to RV infections and are associated with increased airway inflammation; increased mucus production, delayed or deficient antiviral host defenses, and increases in pathogenic respiratory bacteria.

In a RV challenge model in subjects with COPD, Mallia et al. demonstrated that RV infection induces the symptomatic, physiologic, and inflammatory features reported in naturally occurring COPD exacerbations, and that infection is associated with impaired IFN production and exaggerated neutrophilic inflammation, which may be important mechanisms for viral-induced exacerbations. This study also showed upper respiratory symptoms began shortly after viral challenge, with peak lower respiratory symptoms, neutrophil inflammation, and secondary bacterial infection following at time points >1 week from onset of initial symptoms. This progressive time course suggests a potential treatment window for interruption of acute COPD exacerbations associated with RVs by using potent specific antiviral treatments.

Vapendavir (3-Ethoxy-6-{2-[1-(6-methylpyridazin-3-yl)piperidin-4-yl]ethoxy}-benzo[d]isoxazol) is a potent and broad spectrum antiviral agent active against >97% of RV-A and RV-B serotypes of RV (no available assay for RV-C, but clinical data indicate sensitivity similar to A and B) and 89% of other EVs evaluated in cell based assay systems. Vapendavir acts by binding to the viral capsid, thereby inhibiting viral attachment to the target cell and, independently, preventing release of viral RNA into the cell. Vapendavir has been in development for the treatment of infections with RV and other respiratory EVs for approximately a decade, with over 640 healthy adults and participants with asthma receiving treatment across 7 clinical studies. Vapendavir reduced RV viral load compared with placebo in three Phase 2 clinical trials and reduced respiratory symptoms in a Phase 2 clinical trial, both in adult participants with asthma. An acceptable safety and tolerability profile has been demonstrated to date, with no appreciable safety concerns noted following single doses up to 1,056 milligram (mg) and multiple doses up to 528 mg bis in de/twice a day (BID) for up to 7 days, and 400 mg BID, up to 10 days in a RV challenge model. Vapendavir has also been tested against the RV-A16 human challenge strain and demonstrated complete protection against viral replication and de novo infection at ≥0.312 micromolar (μM) using in vitro HeLa cell assays. Vapendavir may also be referred to as VPV.

In some embodiments, the present disclosure describes a sulfate salt of a compound of Formula I:

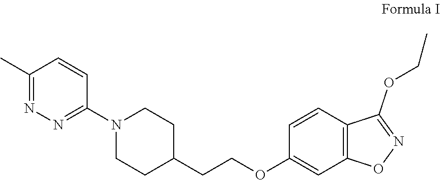 [SMILES: CCOc1noc2cc(OCCC3CCN(c4ccc(C)nn4)CC3)ccc12]

The sulfate salt of vapendavir (also referred to herein as vapendavir sulfate) may have a molecular weight of about 480 g/mol. Vapendavir free base may have a molecular weight of about 382 g/mol.

The solubility of a compound in solutions of various pH, as well as simulated gastric and intestinal fluid solutions, may, in some embodiments, be useful in determining how the compound will dissolve when administered to a subject, without wishing to be bound by theory. It is contemplated that different crystalline forms of vapendavir sulfate may exhibit different solubilities in various solvents and simulated fluids. In some embodiments, a change in crystalline form was observed in the vapendavir sulfate after evaluation of solubility in simulated gastric and intestinal fluid solutions. For example, after evaluation of solubility in simulated gastric and intestinal fluid solutions, the solution may be removed and residual solids evaluated, and in some embodiments, the vapendavir sulfate is in a different crystalline form than the crystalline form initially used in the solubility studies.

In some embodiments, the sulfate salt of vapendavir has a solubility of about 4.0 mg/mL to about 8.5 mg/mL in a hydrochloric acid buffer solution having a pH of about 1.2. For example, the vapendavir sulfate may have a solubility of about 4.0 mg/mL, about 4.1 mg/mL, about 4.2 mg/mL, about 4.3 mg/mL, about 4.4 mg/mL, about 4.5 mg/mL, about 4.6 mg/mL, about 4.7 mg/mL, about 4.8 mg/mL, about 4.9 mg/mL, about 5.0 mg/mL, about 5.1 mg/mL, about 5.2 mg/mL, about 5.3 mg/mL, about 5.4 mg/mL, about 5.5 mg/mL, about 5.6 mg/mL, about 5.7 mg/mL, about 5.8 mg/mL, about 5.9 mg/mL, about 6.0 mg/mL, about 6.1 mg/mL, about 6.2 mg/mL, about 6.3 mg/mL, about 6.4 mg/mL, about 6.5 mg/mL, about 6.6 mg/mL, about 6.7 mg/mL, about 6.8 mg/mL, about 6.9 mg/mL, about 7.0 mg/mL, about 7.1 mg/mL, about 7.2 mg/mL, about 7.3 mg/mL, about 7.4 mg/mL, about 7.5 mg/mL, about 8.0 mg/mL, about 8.5 mg/mL, or any value contained within a range formed by any two of the preceding values, in a solution having a pH of about 1.2.

In some embodiments, the sulfate salt of vapendavir has a solubility of about 1.0 mg/mL to about 3.0 mg/mL in a Fasted State Simulated Gastric Fluid (FaSSGF) solution, which has a pH of about 1.61, at a temperature of about 37° C. For example, in a FaSSGF solution, the vapendavir sulfate may have a solubility of about 1.0 mg/mL, about 1.5 mg/mL, about 2.0 mg/mL, about 2.1 mg/mL, about 2.2 mg/mL, about 2.3 mg/mL, about 2.4 mg/mL, 2.5 mg/mL, about 2.6 mg/mL, about 2.7 mg/mL, about 2.8 mg/mL, about 2.9 mg/mL, about 3.0 mg/mL, or any value contained within a range formed by any two of the preceding values. In some embodiments, vapendavir sulfate exhibits higher solubility in a FaSSGF solution than vapendavir in a free base form.

In some embodiments, the sulfate salt of vapendavir has a solubility of about 0.10 mg/mL to about 0.20 mg/mL in a citrate buffer solution having a pH of about 3.0, such as about 0.10 mg/mL, about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, about 0.19 mg/mL, about 0.20 mg/mL, or any value contained within a range formed by any two of the preceding values.

In some embodiments, the sulfate salt of vapendavir has a solubility of about 0.030 mg/mL to about 0.050 mg/mL in an acetate buffer solution having a pH of about 4.5, such as about 0.030 mg/mL, about 0.035 mg/mL, about 0.040 mg/mL, about 0.045 mg/mL, about 0.050 mg/mL, or any value contained within a range formed by any two of the preceding values.

In some embodiments, the sulfate salt of vapendavir has a solubility of about 0.030 mg/mL to about 0.060 mg/mL in a Fed State Simulated Intestinal Fluid (FeSSIF) solution, which has a pH of about 5.0, at a temperature of about 37° C. For example, in a FeSSIF solution, the vapendavir sulfate may have a solubility of about 0.030 mg/mL, about 0.035 mg/mL, about 0.040 mg/mL, about 0.045 mg/mL, about 0.050 mg/mL, about 0.055 mg/mL, about 0.060 mg/mL, or any value contained within a range formed by any two of the preceding values. In some embodiments, vapendavir sulfate exhibits higher solubility in a FeSSIF solution than vapendavir in a free base form.

In some embodiments, the sulfate salt of vapendavir has a solubility of about 0.005 mg/mL to about 0.020 mg/mL in a Fasted State Simulated Intestinal Fluid (FaSSIF) solution, which has a pH of about 6.50, at a temperature of about 37° C. For example, in a FaSSIF, the vapendavir sulfate may have a solubility of about 0.005 mg/mL, about 0.010 mg/mL, about 0.015 mg/mL, about 0.020 mg/mL, or any value contained within a range formed by any two of the preceding values. In some embodiments, vapendavir sulfate exhibits higher solubility in a FaSSIF solution than vapendavir in a free base form.

In some embodiments, the sulfate salt of vapendavir has a solubility of about 0.0002 mg/mL to about 0.0006 mg/mL in a phosphate buffer solution having a pH of about 6.8, such as about 0.0002 mg/mL, about 0.0003 mg/mL, about 0.0004 mg/mL, about 0.0005 mg/mL, about 0.0006 mg/mL, or any value contained within a range formed by any two of the preceding values.

In some embodiments, the sulfate salt of vapendavir has a solubility of about 0.0002 mg/mL to about 0.0006 mg/mL in a phosphate buffer solution having a pH of about 7.4 at a temperature of about 37° C. For example, in a solution having a pH of about 7.4, the vapendavir sulfate may have a solubility of about 0.0002 mg/mL, about 0.0003 mg/mL, about 0.0004 mg/mL, about 0.0005 mg/mL, about 0.0006 mg/mL, or any value contained within a range formed by any two of the preceding values. In some embodiments, vapendavir sulfate exhibits higher solubility in a phosphate buffer solution than vapendavir in a free base form.

In some embodiments, the sulfate salt of vapendavir has a solubility of about 0.0002 mg/mL to about 0.0006 mg/mL in a phosphate buffer solution having a pH of about 8.0, such as about 0.0002 mg/mL, about 0.0003 mg/mL, about 0.0004 mg/mL, about 0.0005 mg/mL, about 0.0006 mg/mL, or any value contained within a range formed by any two of the preceding values.

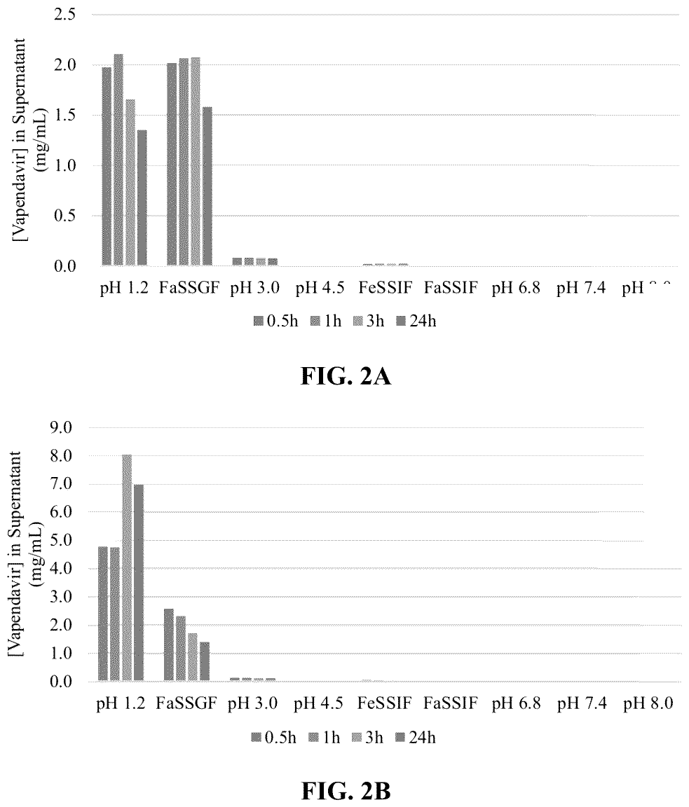

FIG. 2A is a bar graph showing the equilibrium solubility of vapendavir free base in FaSSIF, FeSSIF, FaSSGF and solutions of various pH. FIG. 2B is a bar graph showing the equilibrium solubility of vapendavir bisulfate in FaSSIF, FeSSIF, FaSSGF, and solutions of various pH, according to an embodiment of the present disclosure. As shown in FIG. 2A and FIG. 2B, vapendavir bisulfate exhibits higher solubility than vapendavir free base, particularly in low-pH solutions such as hydrochloric acid buffer having a pH of 1.2 and FaSSGF.

It is contemplated that the vapendavir sulfate may exhibit some solubility in various solvents and solvent combinations, including but not limited to methanol, isopropyl alcohol, tetrahydrofuran, water, or combinations thereof. For example, the vapendavir may exhibit a solubility of greater than about 9 mg/mL in methanol at 25° C., a solubility of greater than about 15 mg/mL in a mixture of isopropyl alcohol and water at 25° C., and a solubility of greater than about 20 mg/mL in a mixture of methanol and water at 25° C.

In some embodiments, a method of preparing vapendavir sulfate can include dissolving vapendavir, in a free base form, in a solvent such as tetrahydrofuran and adding a sulfate-containing compound, such as sulfuric acid. Other methods of preparing vapendavir sulfate may also be acceptable.

In some embodiments, the chemical purity of the vapendavir sulfate may be determined by an analytical method, including but not limited to high performance liquid chromatography (HPLC). In some embodiments, the vapendavir sulfate has a chemical purity of greater than or equal to about 95% by mass, such as about 95%, about 96%, about 97%, about 98%, about 99%, about 100%, or any value contained within a range formed by any two of the preceding values. In some embodiments, the vapendavir sulfate contains less than or equal to about 5% by mass of chemical impurities. The identity or identities of the chemical impurities which may be included is not limited. Chemical impurities may be identified and quantified by any method or technique available to those skilled in the art. Chemical impurities may be identified, reduced, or removed by methods available and familiar to those skilled in the art.

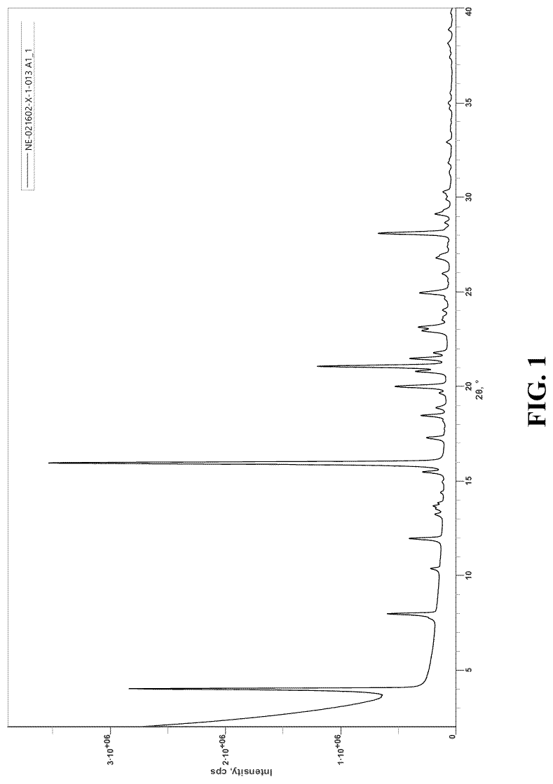

In some embodiments, the polymorphic purity of the vapendavir sulfate may be evaluated by an analytical method such as quantitative XRPD. In some embodiments, the polymorphic purity of the vapendavir sulfate may be determined by evaluating the XRPD pattern and observing the absence of characteristic, non-overlapping peaks of other known crystal forms, and if no such peaks are present, the vapendavir sulfate is considered to be polymorphically pure within the limit of detection of the instrument used to perform XRPD. In some embodiments, the vapendavir sulfate as described herein has a polymorphic purity of at least about 50%, at least about 60%, at least about 70%, at least about 80%, at least about 90%, at least about 95%, or any value contained within a range formed by any two of the preceding values. For example, in some embodiments, the vapendavir sulfate may have a polymorphic purity of at least about 90%, meaning that at least about 90% by mass of the crystalline vapendavir sulfate is a particular crystalline form, such as Crystal Form A. FIG. 1 is an XRPD diffractogram of Crystalline Form A of vapendavir bisulfate, according to embodiments the present disclosure.

### Pharmaceutical Compositions

There is provided, in some embodiments, a pharmaceutical composition which includes a therapeutically effective amount of vapendavir sulfate as described herein. The vapendavir sulfate which is included in the pharmaceutical composition may be in a crystalline form, an amorphous form, or combinations thereof. The vapendavir sulfate which is included in the pharmaceutical composition may be in any of the crystalline forms or combinations thereof as disclosed herein. In some embodiments, the pharmaceutical composition includes vapendavir sulfate which is in a crystalline form having an X-ray powder diffraction (XRPD) pattern which exhibits peaks at diffraction angles of 4.0°, 8.0°, 15.9°, 20.0°, 21.0°, and 28.1° (2θ+0.2°).

In some embodiments, the particle size distribution of the vapendavir sulfate may be measured using techniques and equipment available to those skilled in the art, including laser diffraction instruments.

In some embodiments, the vapendavir sulfate may have a particle size distribution with a D90 of about 35 μm to about 140 μm, such as about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, or any value contained within a range formed by any two of the preceding values. In some embodiments, the vapendavir sulfate may have a particle size distribution with a D50 of about 10 μm to about 25 μm, such as about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, or any value contained within a range formed by any two of the preceding values. In some embodiments, the vapendavir sulfate may have a particle size distribution with a D10 of about 2.5 μm to about 10 μm, such as about 2.5 μm, about 3.0 μm, about 3.5 μm, about 4.0 μm, about 4.5 μm, about 5.0 μm, about 5.5 μm, about 6.0 μm, about 6.5 μm, about 7.0 μm, about 7.5 μm, about 8.0 μm, about 8.5 μm, about 9.0 μm, about 9.5 μm, about 10.0 μm, or any value contained within a range formed by any two of the preceding values. In some embodiments, the vapendavir sulfate has a particle size distribution with a D90 of about 35 μm to about 140 μm, a D50 of about 10 μm to about 25 μm, and a D10 of about 2.5 μm to about 10 μm. In some embodiments, the vapendavir sulfate has a particle size distribution with a D90 of about 100 μm, a D50 of about 25 μm, and a D10 of about 10 μm.

The compounds of the disclosure may be formulated for administration in any convenient way for use in human or veterinary medicine and the disclosure therefore includes within its scope pharmaceutical compositions comprising a compound of the disclosure adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may include, in addition to the carrier, any suitable binder(s), lubricant(s), suspending agent(s), coating agent(s), and/or solubilizing agent(s).

The pharmaceutical compositions can be administered orally in the form of tablets containing excipients such as starch or lactose, or in capsules or ovules either alone or in admixture with excipients, or in the form of elixirs, solutions, or suspensions containing flavoring or coloring agents, or they can be injected parenterally, for example intravenously, intramuscularly, or subcutaneously. For buccal or sublingual administration, the compositions may be administered in the form of tablets or lozenges, which can be formulated in a conventional manner.

The pharmaceutical compositions of the present disclosure can be administered in the form of tablets, capsules, troches, ovules, elixirs, solutions, or suspensions, for immediate-, delayed-, modified-, sustained-, pulsed-, or controlled-release applications. The pharmaceutical compositions of the present disclosure may also be presented in the form of solutions, gels, syrups, or suspensions, or a dry powder for reconstitution with water or other suitable vehicle before use. Solid compositions such as tablets, capsules, lozenges, troches, pastilles, pills, boluses, powder, pastes, granules, bullets, or premix preparations may also be used. Solid and liquid compositions for oral use may be prepared according to methods well-known in the art. Such compositions may also contain one or more pharmaceutically acceptable carriers and excipients which may be in solid or liquid form.

The pharmaceutical composition may, in some embodiments, include vapendavir sulfate in an amount sufficient to provide about 200 mg to about 2000 mg of vapendavir free base. For example, in some embodiments, the pharmaceutical composition includes vapendavir sulfate in an amount sufficient to provide about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1,000 mg, about 1100 mg, about 1200 mg, about 1300 mg, about 1400 mg, about 1500 mg, about 1600 mg, about 1700 mg, about 1800 mg, about 1900 mg, or about 2000 mg of vapendavir free base, or any value contained within a range formed by any two of the preceding values. In some embodiments, the pharmaceutical composition includes about 250 mg to about 2500 mg of vapendavir sulfate, such as about 250 mg, about 500 mg, about 750 mg, about 1000 mg, about 1500 mg, about 2000 mg, about 2500 mg, or any value contained within a range formed by any two of the preceding values. In some embodiments, the pharmaceutical composition is a tablet. The pharmaceutical composition may further include a diluent, a disintegrant, a surfactant, a lubricant, a color coating, or any combination thereof.

Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents, and coatings. Some excipients may have multiple roles in the compositions, that is, act as both binders and disintegrants.

In some embodiments, the diluent includes microcrystalline cellulose (such as Cellulose Microcrystalline Silicified (PROSOLVHD90) (USP/NF)), anhydrous calcium phosphate, anhydrous lactose, calcium carbonate, calcium lactate, calcium sulfate dihydrate, corn starch, fructose, kaolin, lactose monohydrate, magnesium hydroxide, maltitol, maltose monohydrate, mannitol, sorbitol, sucrose, tribasic calcium phosphate, sodium citrate, glycine, croscarmellose sodium, ethanol, propylene glycol, glycerin, polyvinylpyrrolidone, hydroxypropylmethylcellulose (HPMC), hydroxypropylcellulose (HPC), sucrose, gelatin, acacia, or any combination thereof.

In some embodiments, the disintegrant includes Starch Glycolate Sodium (USP/NF/EP), corn starch, potato starch, tapioca starch, croscarmellose sodium, pre-gelatinized starch, sodium carboxymethylcellulose, alginates, resins, aqueous aluminum silicates, cross-linked polyvinylpyrrolidone, or any combination thereof.

In some embodiments, the surfactant includes Poloxamer Microprilled 188 (USP/NF/EP/JPE), sodium lauryl sulfate, polysorbates, or any combination thereof.

In some embodiments, the lubricant includes Magnesium Stearate 5712 (USP/NF/EP), stearic acid, glyceryl behenate, talc, carnauba wax, polymers of ethylene oxide, sodium lauryl sulfate, magnesium lauryl sulfate, sodium oleate, sodium stearyl fumarate, colloidal silicon dioxide, hydroxypropylmethylcellulose, hydroxypropylcellulose, acrylate-methacrylate copolymers, or any combination thereof.

In some embodiments, the color coating includes Opadry II White 85F18422 (Mfg. Std.), riboflavin, anthocyanin, paprika oleoresin, beet root, curcumin, indigo carmine, tartrazine, allura red, quinoline yellow, titanium dioxide, iron oxide, mica, betacarotene, or any combination thereof.

In some embodiments, the pharmaceutical composition includes vapendavir sulfate as described herein and further includes Cellulose Microcrystalline Silicified (PROSOLVHD90) (USP/NF), Starch Glycolate Sodium (USP/NF/EP), Poloxamer Microprilled 188 (USP/NF/EP/JPE), Magnesium Stearate 5712 (USP/NF/EP), and Opadry II White 85F18422 (Mfg. Std.).

In some embodiments, the pharmaceutical composition includes a diluent, such as Cellulose Microcrystalline Silicified (PROSOLVHD90) (USP/NF). In some embodiments, the pharmaceutical composition includes about 250 mg of a diluent, such as Cellulose Microcrystalline Silicified (PROSOLVHD90) (USP/NF). For example, the pharmaceutical composition may include about 200 mg, about 210 mg, about 220 mg, about 230 mg, about 240 mg, about 250 mg, about 260 mg, about 270 mg, about 280 mg, about 290 mg, about 300 mg of the diluent, or any value contained within a range formed by any two of the preceding values. In some embodiments, the pharmaceutical composition includes about 500 mg of a diluent, such as Cellulose Microcrystalline Silicified (PROSOLVHD90) (USP/NF). For example, the pharmaceutical composition may include about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg of the diluent, or any value contained within a range formed by any two of the preceding values. of the diluent, or any value contained within a range formed by any two of the preceding values.

In some embodiments, the pharmaceutical composition includes a disintegrant, such as Starch Glycolate Sodium (USP/NF/EP). In some embodiments, the pharmaceutical composition includes about 50 mg of a disintegrant, such as Starch Glycolate Sodium (USP/NF/EP). For example, the pharmaceutical composition may include about 40 mg, about 45 mg, about 50 mg, about 55 mg, about 60 of the disintegrant, or any value contained within a range formed by any two of the preceding values. In some embodiments, the pharmaceutical composition includes about 100 mg of a disintegrant, such as Starch Glycolate Sodium (USP/NF/EP). For example, the pharmaceutical composition may include about 80 mg, about 90 mg, about 100 mg, about 110 mg, about 120 of the disintegrant, or any value contained within a range formed by any two of the preceding values.

In some embodiments, the pharmaceutical composition includes a surfactant, such as Poloxamer Microprilled 188 (USP/NF/EP/JPE). In some embodiments, the pharmaceutical composition includes about 10 mg of a surfactant, such as Poloxamer Microprilled 188 (USP/NF/EP/JPE). For example, the pharmaceutical composition may include about 8 mg, about 9 mg, about 10 mg, about 11 mg, about 12 mg of the surfactant, or any value contained within a range formed by any two of the preceding values. In some embodiments, the pharmaceutical composition includes about 20 mg of a surfactant, such as Poloxamer Microprilled 188 (USP/NF/EP/JPE). For example, the pharmaceutical composition may include about 16 mg, about 18 mg, about 20 mg, about 22 mg, about 24 mg of the surfactant, or any value contained within a range formed by any two of the preceding values.

In some embodiments, the pharmaceutical composition includes a lubricant, such as Magnesium Stearate 5712 (USP/NF/EP). In some embodiments, the pharmaceutical composition includes about 10 mg of a lubricant, such as Magnesium Stearate 5712 (USP/NF/EP). For example, the pharmaceutical composition may include about 8 mg, about 9 mg, about 10 mg, about 11 mg, about 12 mg of the lubricant, or any value contained within a range formed by any two of the preceding values. In some embodiments, the pharmaceutical composition includes about 20 mg of a lubricant, such as Magnesium Stearate 5712 (USP/NF/EP). For example, the pharmaceutical composition may include about 16 mg, about 18 mg, about 20 mg, about 22 mg, about 24 mg of the lubricant, or any value contained within a range formed by any two of the preceding values.

In some embodiments, the pharmaceutical composition includes a color coating, such as Opadry II White 85F18422 (Mfg. Std.). In some embodiments, the pharmaceutical composition includes about 15 mg of a color coating, such as Opadry II White 85F18422 (Mfg. Std.). For example, the pharmaceutical composition may include about 12 mg, about 13 mg, about 14 mg, about 15 mg, about 16 mg, about 17 mg, about 18 mg of the color coating, or any value contained within a range formed by any two of the preceding values. In some embodiments, the pharmaceutical composition includes about 15 mg of a color coating, such as Opadry II White 85F18422 (Mfg. Std.). For example, the pharmaceutical composition may include about 24 mg, about 26 mg, about 28 mg, about 30 mg, about 32 mg, about 34 mg, about 36 mg of the color coating, or any value contained within a range formed by any two of the preceding values.

In some embodiments, the pharmaceutical composition includes about 250 mg of vapendavir or a sulfate salt thereof, about 250 mg of Cellulose Microcrystalline Silicified (PROSOLVHD90) (USP/NF), about 50 mg of Starch Glycolate Sodium (USP/NF/EP), about 10 mg of Poloxamer Microprilled 188 (USP/NF/EP/JPE), about 10 mg of Magnesium Stearate 5712 (USP/NF/EP), and about 15 mg of Opadry II White 85F18422 (Mfg. Std.).

In some embodiments, the pharmaceutical composition includes about 500 mg of vapendavir or a sulfate salt thereof, about 500 mg of Cellulose Microcrystalline Silicified (PROSOLVHD90) (USP/NF), about 100 mg of Starch Glycolate Sodium (USP/NF/EP), about 20 mg of Poloxamer Microprilled 188 (USP/NF/EP/JPE), about 20 mg of Magnesium Stearate 5712 (USP/NF/EP), and about 30 mg of Opadry II White 85F18422 (Mfg. Std.).

In some embodiments, there is provided a pharmaceutical composition, such as a solid oral composition, including: a therapeutically effective amount, such as about 250 mg to about 2500 mg, of Crystalline Form A of vapendavir sulfate; and one or more pharmaceutically acceptable excipients, such as a diluent, a disintegrant, a surfactant, a lubricant, a color coating, or any combination thereof.

In some embodiments, there is provided a solid oral dosage composition which includes a therapeutically effective amount of vapendavir sulfate and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%. As disclosed herein, chemical purity may be determined by methods familiar to those skilled in the art such as HPLC and polymorphic purity may be determined by, for example, quantitative XRPD.

In some embodiments, there is provided a solid oral dosage composition which includes vapendavir sulfate in an amount sufficient to provide about 100 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 200 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 300 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 400 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 500 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 600 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 700 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 800 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 900 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a solid oral dosage composition which includes vapendavir sulfate in an amount sufficient to provide about 1000 mg of vapendavir free base and one or more pharmaceutically acceptable excipients, wherein the vapendavir sulfate has a chemical purity of at least about 95%, wherein the vapendavir sulfate is in the form of Crystalline Form A, and wherein the vapendavir sulfate has a polymorphic purity of at least about 90%.

In some embodiments, there is provided a pharmaceutical composition which includes a therapeutically effective amount of vapendavir sulfate and one or more pharmaceutically acceptable excipients, wherein the vapendavir sulfate is in the form of Crystalline Form A, wherein the vapendavir sulfate has a particle size distribution with a D90 of about 35 μm to about 140 μm, a D50 of about 10 μm to about 25 μm, and a D10 of about 2.5 μm to about 10 μm, and wherein the pharmaceutical composition is a solid oral dosage form. In some embodiments, the therapeutically effective amount of vapendavir sulfate is about 100 mg to about 1000 mg, such as about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, or any value contained within a range formed by any two of the preceding values. In some embodiments, the vapendavir sulfate has a polymorphic purity of at least about 90%. In some embodiments, the vapendavir sulfate has a particle size distribution with a D90 of about 100 μm, a D50 of about 25 μm, and a D10 of about 10 μm.

### Methods of Treatment

In some embodiments, there is provided a method of treating a respiratory enterovirus infection in a human subject with COPD, with includes orally administering to the subject a therapeutically effective amount of vapendavir sulfate, wherein the respiratory enterovirus infection is treated.

In some embodiments, there is provided a method of treating a respiratory condition, wherein the respiratory condition is a respiratory enterovirus infection, an upper respiratory infection, a common cold, or combinations thereof, in a human subject with COPD, with includes orally administering to the subject a therapeutically effective amount of vapendavir sulfate, wherein the respiratory enterovirus infection is treated; wherein the therapeutically effective amount of vapendavir sulfate is between about 250 mg and about 2,000 mg per day

In some embodiments, the subject has a respiratory enterovirus infection. In some embodiments, the respiratory enterovirus infection is acute, and in other embodiments, the respiratory enterovirus infection is not acute. In some embodiments, the respiratory enterovirus is selected from a rhinovirus, echovirus, EV-68, EV-71, coxsackie virus, a non-polio enterovirus and in some embodiments, the subject has a common cold.

In some embodiments, there is provided a method of treating a patient in need thereof including administering to the patient a pharmaceutical composition, such as a solid oral composition, including: a therapeutically effective amount, such as about 250 mg to about 2000 mg, of vapendavir sulfate; and one or more pharmaceutically acceptable excipients, such as a diluent, a disintegrant, a surfactant, a lubricant, a color coating, or any combination thereof. In some embodiments, the patient has a respiratory condition as described herein.

In some embodiments, the therapeutically effective amount of vapendavir sulfate is between about 250 mg and about 2,000 mg per day, such as about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,650 mg, about 1,700 mg, about 1,750 mg, about 1,800 mg, about 1,850 mg, about 1,900 mg, about 1,950 mg, about 2,000 mg, or any range or value contained within these values.

In some embodiments, the therapeutically effective amount of vapendavir sulfate is a loading dose followed by one or more maintenance doses. The loading dose may be an initial dose on the first day of administration, and the maintenance doses may be administered once daily, twice daily, or at other intervals thereafter. In some embodiments, the loading dose of vapendavir sulfate can be about 600 mg to about 1500 mg, such as about 600 mg, about 675 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, or any value contained within a range formed by two of the preceding values. In some embodiments, the maintenance dose of vapendavir sulfate is about 250 mg to about 750 mg, such as about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, or any value contained within a range formed by two of the preceding values.

In some embodiments, the therapeutically effective amount of vapendavir sulfate is a loading dose of about 600 mg followed by one or more maintenance doses of about 250 mg. In some embodiments, the therapeutically effective amount of vapendavir sulfate is a loading dose of about 700 mg followed by one or more maintenance doses of about 350 mg. In some embodiments, the therapeutically effective amount of vapendavir sulfate is a loading dose of about 700 mg followed by one or more maintenance doses of about 500 mg. In some embodiments, the therapeutically effective amount of vapendavir sulfate is a loading dose of about 750 mg followed by one or more maintenance doses of about 500 mg. In some embodiments, the therapeutically effective amount of vapendavir sulfate is a loading dose of about 800 mg followed by one or more maintenance doses of about 300 mg. In some embodiments, the therapeutically effective amount of vapendavir sulfate is a loading dose of about 800 mg followed by one or more maintenance doses of about 400 mg. In some embodiments, the therapeutically effective amount of vapendavir sulfate is a loading dose of about 800 mg followed by one or more maintenance doses of about 500 mg. In some embodiments, the therapeutically effective amount of vapendavir sulfate is a loading dose of about 900 mg followed by one or more maintenance doses of about 500 mg. In some embodiments, the therapeutically effective amount of vapendavir sulfate is a loading dose of about 1000 mg followed by one or more maintenance doses of about 300 mg. In some embodiments, the therapeutically effective amount of vapendavir sulfate is a loading dose of about 1000 mg followed by one or more maintenance doses of about 500 mg.

In some embodiments, the therapeutically effective amount of vapendavir sulfate is between about 1 mg/kg and about 75 mg/kg, relative to the body weight of the subject. For example, the therapeutically effective amount of vapendavir sulfate is, in some embodiments, about 1 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or any range or value contained therein. In some embodiments, the therapeutically effective amount of vapendavir sulfate is about 14 mg/kg. In some embodiments, the therapeutically effective amount of vapendavir sulfate is about 7 mg/kg. In some embodiment, the therapeutically effective amount of vapendavir sulfate is about 3.5 mg/kg.

In some embodiments, the therapeutically effective amount of vapendavir sulfate is an amount that achieves a Cmax of about 2000 to about 12000 ng/mL. In some embodiments, the therapeutically effective amount of vapendavir sulfate is an amount that achieves a Cmax of about 2000 to about 6000 ng/mL. In some embodiments, the therapeutically effective amount of vapendavir sulfate is an amount that achieves a Cmax of about 2000 to about 4000 ng/mL. In some embodiments, the therapeutically effective amount of vapendavir sulfate is an amount that achieves a Cmax of about 3000 to about 6000 ng/mL. In some embodiments, the therapeutically effective amount of vapendavir sulfate is an amount that achieves a Cmax of about 6000 to about 8000 ng/mL. In some embodiments, the therapeutically effective amount of vapendavir sulfate is an amount that achieves a Cmax of about 8000 to about 10000 ng/mL. In some embodiments, the therapeutically effective amount of vapendavir sulfate is an amount that achieves a Cmax of about 10000 to about 12000 ng/mL.

In some embodiments, the therapeutically effective amount of vapendavir sulfate is a single loading dose of about 500 mg to about 1,000 mg, such as about 500 mg, about 600 mg, about 700 mg, about 800 mg, about 900 mg, about 1000 mg, or any value contained within a range formed by any two of the preceding values, followed by one or more maintenance doses, wherein the maintenance dose is smaller than the loading dose.

In some embodiments, the therapeutically effective amount of vapendavir sulfate is a single loading dose of about 1,000 mg followed by a maintenance dose of about 500 mg about every 12 hours. In some embodiments, the therapeutically effective amount of vapendavir sulfate is a single loading dose of about 1,000 mg followed by a maintenance dose of about 500 mg about every 12 hours for about 4 to about 10 days. In some embodiments, the therapeutically effective amount of vapendavir sulfate is a single loading dose of about 1,000 mg followed by a maintenance dose of about 500 mg about every 12 hours for about 5 to about 8 days. In some embodiments, the therapeutically effective amount of vapendavir sulfate is a single loading dose of about 1,000 mg followed by a maintenance dose of about 500 mg every 12 hours for about 6 to about 8 days. In some embodiments, the therapeutically effective amount of vapendavir sulfate is a single loading dose of about 1,000 mg followed by a maintenance dose of about 500 mg every 12 hours for about 7 days.

In some embodiments, the therapeutically effective amount of vapendavir sulfate is a single loading dose of about 1,000 mg followed by a maintenance dose of about 500 mg every 12 hours. In some embodiments, the therapeutically effective amount of vapendavir sulfate is a single loading dose of about 1,000 mg followed by a maintenance dose of about 500 mg every 12 hours for about 4 to about 10 days. In some embodiments, the therapeutically effective amount of vapendavir sulfate is a single loading dose of about 1,000 mg followed by a maintenance dose of about 500 mg every 12 hours for about 5 to about 8 days. In some embodiments, the therapeutically effective amount of vapendavir sulfate is a single loading dose of about 1,000 mg followed by a maintenance dose of about 500 mg every 12 hours for about 6 to about 8 days. In some embodiments, the therapeutically effective amount of vapendavir sulfate is a single loading dose of about 1,000 mg followed by a maintenance dose of about 500 mg every 12 hours for about 7 days.

In some embodiments, the therapeutically effective amount of vapendavir sulfate is a single loading dose of about 1000 mg followed by a maintenance dose of about 500 mg every 12 hours for about 4 days. In some embodiments, the therapeutically effective amount of vapendavir sulfate is a single loading dose of about 1000 mg followed by a maintenance dose of about 500 mg every 12 hours for about 5 days. In some embodiments, the therapeutically effective amount of vapendavir sulfate is a single loading dose of about 1000 mg followed by a maintenance dose of about 500 mg every 12 hours for about 6 days. In some embodiments, the therapeutically effective amount of vapendavir sulfate is a single loading dose of about 1000 mg followed by a maintenance dose of about 500 mg every 12 hours for about 7 days. In some embodiments, the therapeutically effective amount of vapendavir sulfate is a single loading dose of about 1000 mg followed by a maintenance dose of about 500 mg every 12 hours for about 8 days. In some embodiments, the therapeutically effective amount of vapendavir sulfate is a single loading dose of about 1000 mg every 12 hours for one day followed by a maintenance dose of about 500 mg every 12 hours for about 9 days. In some embodiments, the therapeutically effective amount of vapendavir sulfate is a single loading dose of about 1000 mg followed by a maintenance dose of about 500 mg every 12 hours for 10 days.

In some embodiments, the therapeutically effective amount of vapendavir sulfate is a single loading dose of about 1000 mg followed by a maintenance dose of about 500 mg about every 12 hours for about 4 days. In some embodiments, the therapeutically effective amount of vapendavir sulfate is a single loading dose of about 1000 mg followed by a maintenance dose of about 500 mg about every 12 hours for about 5 days. In some embodiments, the therapeutically effective amount of vapendavir sulfate is a single loading dose of about 1000 mg followed by a maintenance dose of about 500 mg every 12 hours for about 6 days. In some embodiments, the therapeutically effective amount of vapendavir sulfate is a single loading dose of about 1000 mg followed by a maintenance dose of about 500 mg about every 12 hours for about 7 days. In some embodiments, the therapeutically effective amount of vapendavir sulfate is a single loading dose of about 1000 mg followed by a maintenance dose of about 500 mg about every 12 hours for about 8 days. In some embodiments, the therapeutically effective amount of vapendavir sulfate is a single loading dose of about 1000 mg per day followed by a maintenance dose of about 500 mg about every 12 hours for about 9 days. In some embodiments, the therapeutically effective amount of vapendavir sulfate is a single loading dose of about 1000 mg followed by a maintenance dose of about 500 mg about every 12 hours for 10 days.

In some embodiments, the therapeutically effective amount of vapendavir sulfate is a single loading dose of about 1,000 mg followed a maintenance dose of about 500 mg every 12 hours for a total of about 13 maintenance doses. In some embodiments, the therapeutically effective amount of vapendavir sulfate is a single loading dose of about 1,000 mg followed a maintenance dose of about 500 mg about every 12 hours for a total of about 13 maintenance doses. In some embodiments, the therapeutically effective amount of vapendavir sulfate is a single loading dose of about 1,000 mg followed a maintenance dose of about 500 mg about every 12 hours for a total of 13 maintenance doses. In some embodiments, the therapeutically effective amount of vapendavir sulfate is a single loading dose of about 1,000 mg followed a maintenance dose of about 500 mg every 12 hours for a total of 13 maintenance doses.

In some embodiments, the therapeutically effective amount of vapendavir sulfate is a single loading dose of about 1000 mg followed by about 10 to about 15 maintenance doses as described herein, such as about 10, about 11, about 12, about 13, about 14, about 15 maintenance doses as described herein.

In some embodiments, the therapeutically effective amount of vapendavir sulfate is a single loading dose of about 14 mg/kg for one day followed by a maintenance dose of about 7 mg/kg every 12 hours for about 4 to about 10 days. In some embodiments, the therapeutically effective amount of vapendavir sulfate is a single loading dose of about 14 mg/kg for one day followed by a maintenance dose of about 7 mg/kg every 12 hours for about 4 days. In some embodiments, the therapeutically effective amount of vapendavir sulfate is a single loading dose of about 14 mg/kg for one day followed by a maintenance dose of about 7 mg/kg every 12 hours for about 5 days. In some embodiments, the therapeutically effective amount of vapendavir sulfate is a single loading dose of about 14 mg/kg for one day followed by a maintenance dose of about 7 mg/kg every 12 hours for about 6 days. In some embodiments, the therapeutically effective amount of vapendavir sulfate is a single loading dose of about 14 mg/kg for one day followed by a maintenance dose of about 7 mg/kg every 12 hours for about 7 days. In some embodiments, the therapeutically effective amount of vapendavir sulfate is a single loading dose of about 14 mg/kg for one day followed by a maintenance dose of about 7 mg/kg every 12 hours for about 8 days. In some embodiments, the therapeutically effective amount of vapendavir sulfate is a single loading dose of about 14 mg/kg for one day followed by a maintenance dose of about 7 mg/kg every 12 hours for about 9 days. In some embodiments, the therapeutically effective amount of vapendavir sulfate is a single loading dose of about 14 mg/kg for one day followed by a maintenance dose of about 7 mg/kg every 12 hours for about 10 days. In some embodiments, the therapeutically effective amount of vapendavir sulfate is a single loading dose of about 14 mg/kg for one day followed by a maintenance dose of about 7 mg/kg every 12 hours for a total of about 13 maintenance doses.

In some embodiments, the therapeutically effective amount of vapendavir sulfate is a single loading dose of about 14 mg/kg for about one day followed by a maintenance dose of about 7 mg/kg about every 12 hours for about 4 to about 10 days. In some embodiments, the therapeutically effective amount of vapendavir sulfate is a single loading dose of about 14 mg/kg for about one day followed by a maintenance dose of about 7 mg/kg about every 12 hours for about 4 days. In some embodiments, the therapeutically effective amount of vapendavir sulfate is a single loading dose of about 14 mg/kg for about one day followed by a maintenance dose of about 7 mg/kg about every 12 hours for about 5 days. In some embodiments, the therapeutically effective amount of vapendavir sulfate is a single loading dose of about 14 mg/kg for about one day followed by a maintenance dose of about 7 mg/kg about every 12 hours for about 6 days. In some embodiments, the therapeutically effective amount of vapendavir sulfate is a single loading dose of about 14 mg/kg for about one day followed by a maintenance dose of about 7 mg/kg every 12 hours for about 7 days. In some embodiments, the therapeutically effective amount of vapendavir sulfate is a single loading dose of about 14 mg/kg for about one day followed by a maintenance dose of about 7 mg/kg about every 12 hours for about 8 days. In some embodiments, the therapeutically effective amount of vapendavir sulfate is a single loading dose of about 14 mg/kg for about one day followed by a maintenance dose of about 7 mg/kg about every 12 hours for about 9 days. In some embodiments, the therapeutically effective amount of vapendavir sulfate is a single loading dose of about 14 mg/kg for about one day followed by a maintenance dose of about 7 mg/kg about every 12 hours for about 9 days. In some embodiments, the therapeutically effective amount of vapendavir sulfate is a single loading dose of about 14 mg/kg for about one day followed by a maintenance dose of about 7 mg/kg about every 12 hours for a total of about 13 maintenance doses.

In some embodiments, the dosage regimen of vapendavir sulfate could be varied. For instance, the single loading dose could be adjusted within a range of about 500 mg to about 1500 mg, followed by a maintenance dose of about 250 mg to about 750 mg every 12 hours for about 3 to about 14 days. The specific dosage could be adjusted based on the patient's weight, age, severity of the COPD, and the specific strain of the respiratory enterovirus infection.

In some embodiments, there is provided a method of treating a respiratory enterovirus in a subject in need thereof, which includes administering to the subject a solid oral dosage composition which includes a therapeutically effective amount of vapendavir sulfate and one or more pharmaceutically acceptable excipients, wherein the respiratory enterovirus in the subject is treated. In some embodiments, the respiratory enterovirus is acute, and in other embodiments, the respiratory enterovirus is not acute. In some embodiments, the respiratory enterovirus is selected from a rhinovirus, echovirus, EV-68, EV-71, coxsackie virus, a non-polio enterovirus and combinations thereof.

In some embodiments, there is provided a method of treating a respiratory enterovirus in a subject in need thereof, which includes administering to the subject a solid oral dosage composition which includes a therapeutically effective amount of vapendavir sulfate and one or more pharmaceutically acceptable excipients, wherein the vapendavir sulfate is in the form of Crystalline Form A and wherein the vapendavir sulfate has a polymorphic purity of at least about 90%, wherein the respiratory enterovirus in the subject is treated.

In some embodiments, there is provided a method of treating a respiratory enterovirus in a subject in need thereof, which includes administering to the subject a solid oral dosage composition which includes a therapeutically effective amount of vapendavir sulfate and one or more pharmaceutically acceptable excipients, wherein the vapendavir sulfate is amorphous, wherein the respiratory enterovirus in the subject is treated.

In some embodiments, there is provided a method of treating a respiratory enterovirus in a subject having COPD in need thereof, which includes administering to the subject a solid oral dosage composition which includes a therapeutically effective amount of vapendavir sulfate and one or more pharmaceutically acceptable excipients, wherein the vapendavir sulfate is in the form of Crystalline Form A wherein the vapendavir sulfate has a polymorphic purity of at least about 90%, wherein the respiratory enterovirus in the subject is treated.

In some embodiments, there is provided a method of treating a respiratory enterovirus in a subject having COPD in need thereof, which includes administering to the subject a solid oral dosage composition which includes a therapeutically effective amount of vapendavir sulfate and one or more pharmaceutically acceptable excipients, wherein the vapendavir sulfate is amorphous, wherein the respiratory enterovirus in the subject is treated.

In some embodiments, the therapeutically effective amount of vapendavir sulfate is an amount sufficient to provide between about 250 mg and about 2,500 mg per day, such as about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,650 mg, about 1,700 mg, about 1,750 mg, about 1,800 mg, about 1,850 mg, about 1,900 mg, about 1,950 mg, about 2,000 mg, about 2,050 mg, about 2,100 mg, about 2,150 mg, about 2,200 mg, about 2,250 mg, about 2,300 mg, about 2,350 mg, about 2,400 mg, about 2,450 mg, about 2,500 mg, of vapendavir free base or any value contained within a range formed by any two of the preceding values.

In some embodiments, the therapeutically effective amount of vapendavir sulfate is an amount sufficient to provide between about 1 mg/kg and about 75 mg/kg of vapendavir free base, relative to the body weight of the subject. For example, the therapeutically effective amount of vapendavir sulfate is, in some embodiments, an amount sufficient to provide about 1 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or any value contained within a range formed by any two of the preceding values, of vapendavir free base. In some embodiments, the therapeutically effective amount of vapendavir sulfate is an amount sufficient to provide about 14 mg/kg of vapendavir free base. In some embodiments, the therapeutically effective amount of vapendavir sulfate is an amount sufficient to provide about 7 mg/kg of vapendavir free base. In some embodiments, the therapeutically effective amount of vapendavir sulfate is an amount sufficient to provide about 3.5 mg/kg of vapendavir free base.

In some embodiments, the therapeutically effective amount of vapendavir sulfate is a loading dose followed by one or more maintenance doses. In some embodiments, the loading dose of vapendavir sulfate can be an amount sufficient to provide about 600 mg to about 1500 mg, such as about 600 mg, about 675 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, or any value contained within a range formed by two of the preceding values, of vapendavir free base. In some embodiments, the maintenance dose of vapendavir sulfate is an amount sufficient to provide about 250 mg to about 750 mg, such as about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, or any value contained within a range formed by two of the preceding values, of vapendavir free base.

In some embodiments, the therapeutically effective amount of vapendavir sulfate is a loading dose in an amount sufficient to provide about 600 mg of vapendavir free base followed by one or more maintenance doses in an amount sufficient to provide about 250 mg of vapendavir free base. In some embodiments, the therapeutically effective amount of vapendavir sulfate is a loading dose in an amount sufficient to provide about 700 mg of vapendavir free base followed by one or more maintenance doses in an amount sufficient to provide about 350 mg of vapendavir free base. In some embodiments, the therapeutically effective amount of vapendavir sulfate is a loading dose in an amount sufficient to provide about 700 mg of vapendavir free base followed by one or more maintenance doses in an amount sufficient to provide about 500 mg of vapendavir free base. In some embodiments, the therapeutically effective amount of vapendavir sulfate is a loading dose in an amount sufficient to provide about 750 mg followed by one or more maintenance doses in an amount sufficient to provide about 500 mg. In some embodiments, the therapeutically effective amount of vapendavir sulfate is a loading dose in an amount sufficient to provide about 800 mg of vapendavir free base followed by one or more maintenance doses in an amount sufficient to provide about 300 mg of vapendavir free base. In some embodiments, the therapeutically effective amount of vapendavir sulfate is a loading dose in an amount sufficient to provide about 800 mg of vapendavir free base followed by one or more maintenance doses in an amount sufficient to provide about 400 mg of vapendavir free base. In some embodiments, the therapeutically effective amount of vapendavir sulfate is a loading dose in an amount sufficient to provide about 800 mg of vapendavir free base followed by one or more maintenance doses in an amount sufficient to provide about 500 mg of vapendavir free base. In some embodiments, the therapeutically effective amount of vapendavir sulfate is a loading dose in an amount sufficient to provide about 900 mg of vapendavir free base followed by one or more maintenance doses in an amount sufficient to provide about 500 mg of vapendavir free base. In some embodiments, the therapeutically effective amount of vapendavir sulfate is a loading dose in an amount sufficient to provide about 1000 mg of vapendavir free base followed by one or more maintenance doses in an amount sufficient to provide about 300 mg of vapendavir free base. In some embodiments, the therapeutically effective amount of vapendavir sulfate is a loading dose in an amount sufficient to provide about 1000 mg of vapendavir free base followed by one or more maintenance doses in an amount sufficient to provide about 500 mg of vapendavir free base.

In some embodiments, the therapeutically effective amount of vapendavir sulfate is a single loading dose in an amount sufficient to provide about 1000 mg per day for one day followed by a maintenance dose in an amount sufficient to provide about 500 mg twice per day (BID) for about 7 to about 10 days. In some embodiments, the therapeutically effective amount of vapendavir sulfate is a single loading dose in an amount sufficient to provide about 14 mg/kg for one day followed by a maintenance dose in an amount sufficient to provide about 7 mg/kg for about 7 to about 10 days.

In some embodiments, the therapeutically effective amount of vapendavir sulfate is a single loading dose in an amount sufficient to provide about 1000 mg per day for one day followed by a maintenance dose in an amount sufficient to provide about 500 mg once per day (BID) for about 7 to about 10 days. In some embodiments, the therapeutically effective amount of vapendavir sulfate is a single loading dose in an amount sufficient to provide about 14 mg/kg for one day followed by a maintenance dose in an amount sufficient to provide about 7 mg/kg for about 7 to about 10 days.

In some embodiments, the therapeutically effective amount of vapendavir sulfate is a once daily dose in an amount sufficient to provide about 1000 mg per day for 5 to 7 days.

In some embodiments, the therapeutically effective amount of vapendavir sulfate is a single loading dose in an amount sufficient to provide about 1000 mg followed by a maintenance dose in an amount sufficient to provide about 500 mg twice per day (BID) for about 7 to about 10 days. In some embodiments, the therapeutically effective amount of vapendavir sulfate is a single loading dose in an amount sufficient to provide about 1000 mg followed by 12 to 20 maintenance doses in an amount sufficient to provide about 500 mg each, such as 12 maintenance doses, 13 maintenance doses, 14 maintenance doses, 15 maintenance doses, 16 maintenance doses, 17 maintenance doses, 18 maintenance doses, 19 maintenance doses, or 20 maintenance doses. In some embodiments, the maintenance doses are administered about 12 hours apart.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by 12 to 20 maintenance doses in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 600 mg of vapendavir free base followed by 12 to 20 maintenance doses in an amount sufficient to provide about 250 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 600 mg of vapendavir free base followed by 12 to 20 maintenance doses in an amount sufficient to provide about 250 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 600 mg of vapendavir free base followed by 12 to 20 maintenance doses of vapendavir sulfate in an amount sufficient to provide about 250 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 350 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 700 mg of vapendavir free base followed by 12 to 20 maintenance doses of vapendavir sulfate in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having COPD in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is in the form of Crystalline Form A, wherein the vapendavir sulfate has a polymorphic purity of at least about 90%, and wherein the maintenance doses are administered to the subject about 12 hours apart, wherein the respiratory enterovirus in the subject is treated.

In some embodiments, there is provided a method of treating a respiratory enterovirus in a subject having asthma in need thereof, which includes administering to the subject a loading dose of vapendavir sulfate in an amount sufficient to provide about 800 mg of vapendavir free base followed by 12 to 20 maintenance doses of vapendavir sulfate in an amount sufficient to provide about 500 mg each of vapendavir free base, wherein the vapendavir sulfate is amorphous and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the subject is not taking an acid reducer and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the subject is taking an acid reducer and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the subject is in a fed state and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the subject is in a fasted state and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by 12 to 20 maintenance doses of vapendavir sulfate in an amount sufficient to provide about 500 mg each of vapendavir free base, wherein the subject is taking an acid reducer, wherein the subject is in a fasted state, and wherein the maintenance doses are administered to the subject about 12 hours apart, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the vapendavir sulfate is in the form of Crystalline Form A, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the vapendavir sulfate is in the form of Crystalline Form A and wherein the vapendavir sulfate has a polymorphic purity of at least about 90%, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the vapendavir sulfate is amorphous, wherein the respiratory enterovirus in the subject is treated.

In some embodiments, there is provided a method of treating a respiratory enterovirus in a subject having COPD in need thereof, which includes administering to the subject vapendavir sulfate in an amount sufficient to provide about 500 mg of vapendavir free base twice per day for about 7 to 10 days, wherein the respiratory enterovirus in the subject is treated.

In some embodiments, there is provided a method of treating a respiratory enterovirus in a subject having COPD in need thereof, which includes administering to the subject vapendavir sulfate in an amount sufficient to provide about 500 mg of vapendavir free base twice per day for about 7 to 10 days, wherein the vapendavir sulfate is in the form of Crystalline Form A, wherein the respiratory enterovirus in the subject is treated.

In some embodiments, there is provided a method of treating a respiratory enterovirus in a subject having COPD in need thereof, which includes administering to the subject vapendavir sulfate in an amount sufficient to provide about 500 mg of vapendavir free base twice per day for about 7 to 10 days, wherein the vapendavir sulfate is in the form of Crystalline Form A and wherein the vapendavir sulfate has a polymorphic purity of at least about 90%, wherein the respiratory enterovirus in the subject is treated.

In some embodiments, there is provided a method of treating a respiratory enterovirus in a subject having COPD in need thereof, which includes administering to the subject vapendavir sulfate in an amount sufficient to provide about 500 mg of vapendavir free base twice per day for about 7 to 10 days, wherein the vapendavir sulfate is amorphous, wherein the respiratory enterovirus in the subject is treated.

In some embodiments, there is provided a method of treating a respiratory enterovirus in a subject having asthma in need thereof, which includes administering to the subject vapendavir sulfate in an amount sufficient to provide about 500 mg of vapendavir free base twice per day for about 7 to 10 days, wherein the respiratory enterovirus in the subject is treated.

In some embodiments, there is provided a method of treating a respiratory enterovirus in a subject having asthma in need thereof, which includes administering to the subject vapendavir sulfate in an amount sufficient to provide about 500 mg of vapendavir free base twice per day for about 7 to 10 days, wherein the vapendavir sulfate is in the form of Crystalline Form A, wherein the respiratory enterovirus in the subject is treated.

In some embodiments, there is provided a method of treating a respiratory enterovirus in a subject having asthma in need thereof, which includes administering to the subject vapendavir sulfate in an amount sufficient to provide about 500 mg of vapendavir free base twice per day for about 7 to 10 days, wherein the vapendavir sulfate is in the form of Crystalline Form A and wherein the vapendavir sulfate has a polymorphic purity of at least about 90%, wherein the respiratory enterovirus in the subject is treated.

In some embodiments, there is provided a method of treating a respiratory enterovirus in a subject having asthma in need thereof, which includes administering to the subject vapendavir sulfate in an amount sufficient to provide about 500 mg of vapendavir free base twice per day for about 7 to 10 days, wherein the vapendavir sulfate is amorphous, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the subject is not taking an acid reducer, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the subject is taking an acid reducer, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the subject is in a fed state, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the subject is in a fasted state, wherein the respiratory enterovirus in the subject is treated.

In some embodiments, there is provided a method of treating a respiratory enterovirus in a subject in need thereof, which includes administering to the subject vapendavir sulfate in an amount sufficient to provide about 500 mg of vapendavir free base twice per day for about 7 to 10 days, wherein the subject is taking an acid reducer and wherein the subject is in a fasted state, wherein the respiratory enterovirus in the subject is treated.

In some embodiments, there is provided a method of treating a respiratory enterovirus in a subject in need thereof, which includes administering to the subject a loading dose of vapendavir sulfate in an amount sufficient to provide about 1000 mg of vapendavir free base followed by about 5 to about 10 maintenance doses in an amount sufficient to provide about 500 mg each of vapendavir free base, wherein the maintenance doses are administered to the subject about 24 hours apart, wherein the respiratory enterovirus in the subject is treated. In some embodiments, there is provided a method of treating a respiratory enterovirus in a subject in need thereof, which includes administering to the subject a loading dose of vapendavir sulfate in an amount sufficient to provide about 1000 mg of vapendavir free base followed by about 5 to about 10 maintenance doses in an amount sufficient to provide about 500 mg each of vapendavir free base, wherein the maintenance doses are administered once per day. In some embodiments, there is provided a method of treating a respiratory enterovirus in a subject in need thereof, which includes administering to the subject a loading dose of vapendavir sulfate in an amount sufficient to provide about 1000 mg of vapendavir free base followed by maintenance doses in an amount sufficient to provide about 500 mg each of vapendavir free base, wherein the maintenance doses are administered once per day for about 5 days to about 10 days, and wherein the subject is in a fed state when administering the loading dose and the maintenance doses. In some embodiments, the subject is not taking an acid reducer when administering the loading and maintenance doses. In some embodiments, orally administering the loading dose to the subject results in a Cₘₐₓ of about 6400 ng/mL to about 10000 ng/mL of vapendavir, such as about 6400 ng/mL, about 7000 ng/mL, about 8000 ng/mL, about 9000 ng/mL, about 10000 ng/mL, or any value contained within a range formed by any two of the preceding values. In some embodiments, a Tₘₐₓ is about 4 hours. In some embodiments, the subject has COPD. In some embodiments, the subject has asthma.

In some embodiments, there is provided a method of treating a respiratory enterovirus in a subject in need thereof, which includes administering to the subject a loading dose of vapendavir sulfate in an amount sufficient to provide about 1000 mg of vapendavir free base, followed by a maintenance dose of vapendavir sulfate in an amount sufficient to provide about 1000 mg of vapendavir free base once per day for about 5 days to about 10 days, wherein the respiratory enterovirus in the subject is treated. In some embodiments, there is provided a method of treating a respiratory enterovirus in a subject in need thereof, which includes administering to the subject a first dose of vapendavir sulfate in an amount sufficient to provide about 1000 mg of vapendavir free base, followed by an additional dose of vapendavir sulfate in an amount sufficient to, provide about 1000 mg of vapendavir free base once per day for about 5 days to about 10 days, wherein the subject is in a fed state when administering the dose of vapendavir sulfate and wherein the respiratory enterovirus in the subject is treated. In some embodiments, the subject is not taking an acid reducer when administering the loading dose and the maintenance doses. In some embodiments, orally administering the loading dose to the subject results in a Cₘₐₓ of about 6400 ng/mL to about 10000 ng/mL of vapendavir, such as about 6400 ng/mL, about 7000 ng/mL, about 8000 ng/mL, about 9000 ng/mL, about 10000 ng/mL, or any value contained within a range formed by any two of the preceding values. In some embodiments, a Tₘₐₓ is about 4 hours. In some embodiments, the subject has COPD. In some embodiments, the subject has asthma.

In some embodiments, the single loading dose is administered within about 24 hours of the subject displaying symptoms of a respiratory enterovirus infection. In some embodiments, the single loading dose is administered within about 48 hours of the subject displaying symptoms of a respiratory enterovirus infection. In some embodiments, the single loading dose is administered within about 72 hours of the subject displaying symptoms of a respiratory enterovirus infection. In some embodiments, the single loading dose is administered within about 96 hours of the subject displaying symptoms of a respiratory enterovirus infection. In some embodiments, the single loading dose is administered within about 72 hours to about 96 hours of the subject displaying symptoms of a respiratory enterovirus infection. In some embodiments, the single loading dose is administered within about 1 to about 5 days of the subject displaying symptoms of a respiratory enterovirus infection. In some embodiments, the single loading dose is administered within about 1 to about 7 days of the subject displaying symptoms of a respiratory enterovirus infection. In some embodiments, the single loading dose is administered greater than 48 hours after the subject first displays symptoms of a respiratory enterovirus infection. In some embodiments, the single loading dose is administered prior to the subject achieving a peak viral load. In some embodiments, the maintenance dose is first administered about 6 to about 12 hours following administering the single loading dose. In some embodiments, the maintenance dose may be administered about 6 hours, about 7 hours, about 8 hours, about 9 hours, about 10 hours, about 11 hours, about 12 hours, or any value contained within a range formed by any two of the preceding values, after administration of the single loading dose. In some embodiments, the at least one maintenance dose is administered for about 7 days to about 10 days, such as about 7 days, about 8 days, about 9 days, or about 10 days.

In some embodiments, the single loading dose and the maintenance dose are administered within about 30 minutes of the subject consuming a meal with solid food (e.g. the subject is in a fed state). In some embodiments, the single loading dose and the maintenance dose are administered within about 30 minutes of the subject consuming a high fat meal with solid food (e.g. the subject is in a fed state). In some embodiments, administering the single loading dose and maintenance dose to a subject within about 30 minutes of the subject consuming a meal with solid food results in a higher plasma concentration of vapendavir sulfate than administering the single loading dose and maintenance dose to a subject in a fasted state. For example, administration of 500 mg vapendavir in normal healthy volunteers that were in a fed state prior to administration results in a Cmax of 2060 ng/mL (CV % 5 ng/mL) compared to 687 ng/mL (CV % 93 ng/mL) in normal healthy volunteers that were in fasted state prior to administration.

In some embodiments, the single loading dose is administered after the subject has been diagnosed with a respiratory enterovirus infection, wherein the diagnosis is achieved by a clinical diagnosis, multiplex PCR testing, rapid antigen testing, direct fluorescent antibody testing, arterial blood gas testing, chest x-rays, peak flow meter testing, pleural fluid cultures, lung CT scanning, lung function tests, pulse oximetry testing, spirometry testing, sputum testing, nasal swab testing, throat swab testing, or any combination thereof.

In some embodiments, the symptoms of a respiratory enterovirus infection are selected from a cough, increased mucus production, sneezing, nasal congestion, runny nose, sore throat, headaches, muscle aches, breathlessness, tight chest, wheezing, fever, red eyes, swelling of the lymph nodes, fatigue, hoarse voice, rhinorrhea, malaise, nasal obstruction, sputum production, inability to function at normal level of activity, and any combinations thereof. In some embodiments, the subject is asymptomatic but has been diagnosed with a respiratory enterovirus infection.

In some embodiments, the subject is a human. In some embodiments, the subject is a human between about 40 and about 85 years of age. In some embodiments, the subject been diagnosed with Global Initiative for Chronic Obstructive Lung Disease (GOLD) stage 1 COPD, GOLD stage 2 COPD, GOLD stage 3 COPD, or GOLD stage 4 COPD. In some embodiments, the subject has been diagnosed with asthma.

In some embodiments, the subject is a female receiving an oral contraceptive, such as birth control. In such embodiments, administering a therapeutically effective amount of vapendavir sulfate further includes administering additional birth control during treatment. In some embodiments, the subject is a female that is not receiving an oral contraceptive, such as birth control.

In some embodiments, the subject is receiving stable maintenance therapy for COPD. In some embodiments, stable maintenance therapy for COPD may include short-acting bronchodilators such as, but not limited to Albuterol, Levalbuterol, Ipratropium, or any combination thereof; long-acting bronchodilators such as, but not limited to Aclidinium, Arformoterol, Formoterol, Glycopyrrolate, Indacaterol, Olodaterol, Revefenacin, Salmeterol, Tiotropium, Umeclidinium, or any combination thereof; corticosteroids such as, but not limited to Fluticasone, Budesonide, Prednisolone, Beclometasone or any combination thereof; LABA and LAMA combination bronchodilator therapies such as, but not limited to Aclidinium/formoterol, Glycopyrrolate/formoterol, Tiotropium/olodaterol, Umeclidinium/vilanterol, Glycopyrronium/indacaterol or any combination thereof; Combinations of an ICS and a long-acting bronchodilator such as, but not limited to Budesonide/formoterol, Fluticasone/salmeterol, Fluticasone/vilanterol, Beclometasone/formoterol or any combination thereof; Triple Combination Therapies (ICS/LAMA/LABA) such as but not limited to Fluticasone/umeclidinium/vilanterol, Budesonide/glycopironium/formoterol, Beclometasone/formoterol/glycopyrronium or any combination thereof; Methylxanthines such as, but not limited to Theophylline; Phosphodiesterase-4 inhibitors such as, but not limited to Roflumilast; Mucoactive drugs such as but not limited to Carbocysteine, Erdosteine, N-acetylcysteine or any combination thereof; Biologics/anti-eosinophilia drugs such as, but not limited to Mepolizumab, Benralizumab, Reslizumab, Dupilumab or any combination thereof; or any other drug prescribed for the treatment of COPD. In some embodiments, the subject may receive the same stable maintenance therapy for COPD than when the subject does not have a respiratory enterovirus infection. In some embodiments, the subject may receive a larger dose, a more frequent dosage, or any combination thereof, of the stable maintenance therapy for COPD than when the subject does not have a respiratory enterovirus infection. In some embodiments, the subject may receive a smaller dose, a less frequent dosage, or any combination thereof, of the stable maintenance therapy for COPD than when the subject does not have a respiratory enterovirus infection. In some embodiments, the method further includes administering a therapeutically effective amount of pleconaril, or another capsid inhibitor, to the subject in combination with vapendavir sulfate.

The subject may, in some embodiments, have had at least one respiratory enterovirus infection in the 12 months immediately prior to administering the therapeutically effective amount of vapendavir sulfate. In some embodiments, the subject has not had a sore throat, sneezing, rhinorrhea, malaise, nasal obstruction, cough, or any combination thereof, in the 30 days prior to administering the therapeutically effective amount of vapendavir sulfate. In some embodiments, the subject does not have an active diagnosed infection or consistent symptoms thereof, with a viral or bacterial pathogen in addition to respiratory enterovirus infection.

In some embodiments, the subject has not been diagnosed with asthma; cystic fibrosis (CF); bronchiolitis obliterans; fibrosis such as tuberculosis (TB), idiopathic pulmonary fibrosis (IPF), other major respiratory diagnosis (e.g., pneumonia, aspergillosis), non-CF bronchiectasis, COPD due to alpha-1 antitrypsin deficiency, active allergic rhinitis, nasal disease, (e.g. nasal polyposis, significant septal deviation, chronic rhinosinusitis, etc.), any cardiovascular, gastrointestinal, hepatic, renal, neurological, musculoskeletal, infectious, endocrine, metabolic, hematological, psychiatric impairment that is not medically stable, or other major physical impairment that is not considered medically stable or controlled, or any combination thereof.

In some embodiments, the subject is not taking any medications that are known to be impacted by or significantly metabolized by CYP3A4. Such medications include but are not limited to Apalutamide, Amiodarone, Carbamazepine, Amprenavir, Dexamethasone, Atazanavir, Enzalutamide, Boceprevir, Fosphenytoin, Clarithromycin, Lumacaftor, Cobicistat, Midostaurin, Conivaptan, Mitotane, Curcumin, Pentobarbital, Danazol, Phenobarbital, Danoprevir, Phenytoin, Darunavir, Primidone, Delavirdine, Rifampicin, Diltiazem, Rifamycin, Ditiocarb, Rifapentine, Econazole, Rifaximin, Efavirenz, Rimexolone, Elvitegravir, St. John's Wort, Ergotamine, Idelalisib, Levoketoconazole, Naloxone, Indinavir, Itraconazole, Ketoconazole, Lonafarnib, Loperamide, Lopinavir, Methimazole, Midostaurin, Nefazodone, Nelfinavir, Nilotinib, Posaconazole, Ribociclib, Ritonavir, Saquinavir, Stiripentol, Telaprevir, Telithromycin, Terfenadine, Tipranavir, Troleandomycin, Voriconazole, or combinations thereof. Such medications may also include H2 blockers such as Famotidine, Cimetidine, Nizatidine and Ranitidine. Such medications may also include proton pump inhibitors such as but not limited to Omeprazole, Esomeprazole, Lansoprazole, Rabeprazole, Pantoprazole, Dexlansoprazole and Zegerid. Such medications may also antacids such a Melox (Aluminum hydroxide, Magnesium hydroxide and Simethicone), Alginate, Aluminum hydroxide, Calcium carbonate, Magnesium carbonate, Magnesium hydroxide, Magnesium trisilicate, and Sodium bicarbonate. In some embodiments, the subject is not taking an acid reducer.

In some embodiments, the subject is not taking an acid reducer, such as a proton pump inhibitor (PPI), a histimine-2 (H2) blocker, an antacid, or a combination thereof. In some embodiments, the subject is taking an acid reducer, such as a proton pump inhibitor (PPI), a histimine-2 (H2) blocker, an antacid, or a combination thereof. In some embodiments, the efficacy of the compositions and methods described herein is not affected by the subject taking an acid reducer, such as a proton pump inhibitor (PPI), a histimine-2 (H2) blocker, an antacid, or a combination thereof, in embodiments wherein the subject is taking an acid reducer. In some embodiments, the subject may be taking an acid reducer and be in a fed or fasted state. In some embodiments, the subject may not be taking an acid reducer and be in a fed or fasted state.

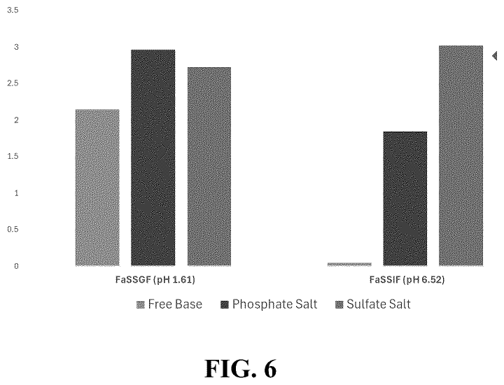

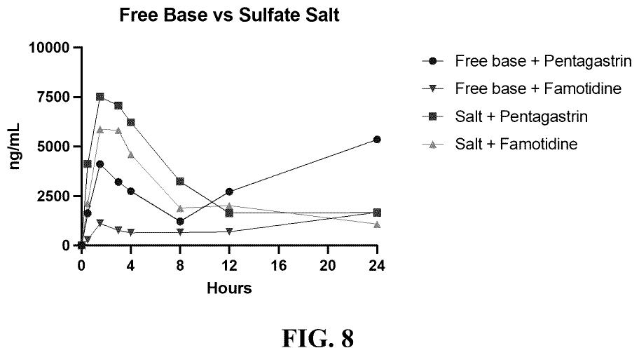

Without wishing to be bound by theory, it is contemplated that the solubility of vapendavir sulfate is less dependent upon pH than vapendavir free base or other salts such as vapendavir phosphate. FIG. 6 is a graph comparing the solubility of vapendavir free base, vapendavir phosphate, and vapendavir sulfate in FaSSGF (pH 1.61) and FaSSIF (pH 6.52) solutions, according to an embodiment of the present disclosure. As shown in FIG. 6, the solubility of vapendavir free base decreases significantly from FaSSGF solution (pH 1.61) to FaSSIF solution (pH 6.52). Vapendavir phosphate similarly shows a decrease in solubility at higher pH. The solubility of vapendavir sulfate, however, remains the same or even increases at higher pH. This unexpected pH-independence is not suggested by prior work on vapendavir free base or other vapendavir salts. The data in FIG. 8 further support this finding. FIG. 8 is a graph of absorption of vapendavir sulfate and vapendavir free base when co-administered with famotidine or pentagastrin, according to an embodiment of the present disclosure. As described further in the Examples, famotidine raises pH while pentagastrin lowers pH, and vapendavir sulfate achieves the same maximum absorption with both famotidine and pentagastrin. This data is supportive of an effective therapy in which vapendavir sulfate can be effectively administered to subjects taking acid reducers without an impact on efficacy, contrary to vapendavir free base and vapendavir phosphate, which have been previously found to be ineffective for subjects on acid reducers. This surprising finding by the inventors of the present disclosure offers a substantial benefit for COPD subjects suffering from respiratory conditions, as COPD subjects often also regularly take acid reducers.

In some embodiments, the method may include administering to the subject a therapeutically effective amount of vapendavir sulfate while the subject is in a fed state. In some embodiments, the method may include administering to the subject a therapeutically effective amount of vapendavir sulfate while the subject is in a fasted state. In some embodiments, any of the methods disclosed herein may include administering to the subject a therapeutically effective amount of vapendavir sulfate regardless of the subject's fed or fasted state. In some embodiments, the method may include administering to the subject a therapeutically effective amount of vapendavir sulfate while the subject is in a fed state and wherein the subject is not taking an acid reducer. In some embodiments, the method may include administering to the subject a therapeutically effective amount of vapendavir sulfate while the subject is in a fasted state and wherein the subject is not taking an acid reducer. In some embodiments, the method may include administering to the subject a therapeutically effective amount of vapendavir sulfate while the subject is in a fed state and wherein the subject is taking an acid reducer. In some embodiments, the method may include administering to the subject a therapeutically effective amount of vapendavir sulfate while the subject is in a fasted state and wherein the subject is taking an acid reducer.

In some embodiments, the subject has no clinically relevant rhinovirus load in nasal lavage or sputum at a baseline visit (such as about 5 to about 10 days prior to beginning treatment, for example about 5 days, about 6 days, about 7 days, about 8 days, about 9 days, or about 10 days), has no clinically relevant rhinovirus load in nasal lavage or sputum at day 0 of treatment, or a combination thereof. In some embodiments, a clinically relevant rhinovirus load may be less than about 20 copies per microliter of cDNA, such as about 20 copies, about 15 copies, about 10 copies, about 5 copies, and so forth, or any value contained within a range formed by any two of the preceding values.

In some embodiments, treating the respiratory condition in the subject includes decreasing and/or preventing an increase in one or more parameters or measurements known to those skilled in the art to be indicative of a subject's health.

In some embodiments, administering the therapeutically effective amount of vapendavir sulfate to the subject results in an individual exposure of about 6000 ng/mL to about 10,000 ng/mL at 4 hours, such as about 6000 ng/mL, about 7000 ng/mL, about 8000 ng/mL, about 9000 ng/mL, about 10,000 ng/mL, or any value contained within a range formed by any two of the preceding values. In some embodiments, administering the therapeutically effective amount of vapendavir sulfate to the subject results in an individual exposure of about 2000 ng/mL to about 6000 ng/mL at 8 hours, such as about 2000 ng/mL, about 3000 ng/mL, about 4000 ng/mL, about 5000 ng/mL, about 6000 ng/mL, or any value contained within a range formed by any two of the preceding values. In some embodiments, administering the therapeutically effective amount of vapendavir sulfate to the subject results in an individual exposure of about 1000 ng/mL to about 6000 ng/mL at 12 hours, such as about 1000 ng/mL, about 2000 ng/mL, about 3000 ng/mL, about 4000 ng/mL, about 5000 ng/mL, about 6000 ng/mL, or any value contained within a range formed by any two of the preceding values.

In some embodiments, administering the therapeutically effective amount of vapendavir sulfate to the subject results in a mean exposure of about 6000 ng/mL to about 8,000 ng/mL at 4 hours, such as about 6000 ng/mL, about 7000 ng/mL, about 8000 ng/mL, or any value contained within a range formed by any two of the preceding values. In some embodiments, administering the therapeutically effective amount of vapendavir sulfate to the subject results in an individual exposure of about 2000 ng/mL to about 4000 ng/mL at 8 hours, such as about 2000 ng/mL, about 3000 ng/mL, about 4000 ng/mL, or any value contained within a range formed by any two of the preceding values. In some embodiments, administering the therapeutically effective amount of vapendavir sulfate to the subject results in an individual exposure of about 1000 ng/mL to about 3000 ng/mL at 12 hours, such as about 1000 ng/mL, about 2000 ng/mL, about 3000 ng/mL, or any value contained within a range formed by any two of the preceding values.

In some embodiments, administering the therapeutically effective amount of vapendavir sulfate to the subject results in an AUC value of about 50,000 ng*h/mL to about 80,000 ng*h/mL, such as about 50,000 ng*h/mL, about 60,000 ng*h/mL, about 70,000 ng*h/mL, about 80,000 ng*h/mL, or any value contained within a range formed by any two of the preceding values.

In some embodiments, administering the therapeutically effective amount of vapendavir sulfate to the subject results in a Cₘₐₓ value of about 5000 ng/mL to about 8500 ng/mL, such as about 5000 ng/mL, about 6000 ng/mL, about 7000 ng/mL, about 8000 ng/mL, about 8500 ng/mL, or any value contained within a range formed by any two of the preceding values.

In some embodiments, treating the respiratory enterovirus in the subject includes a reduction in symptoms, a decrease in viral load in the sputum, nasal passages, or a combination thereof, an improvement in a health parameter of the subject, or combinations thereof.

In some embodiments, treating the respiratory enterovirus infection in the subject includes decreasing a viral load in the sputum, nasal passages, or a combination thereof in the subject compared with a viral load in the sputum, nasal passages, or a combination thereof of the subject prior to treatment. In some embodiments, treating the respiratory enterovirus infection in the subject includes decreasing and/or preventing an increase in one or more parameters or measurements known to those skilled in the art to be indicative of a subject's health.

Treating the respiratory enterovirus infection in the subject may include, in some embodiments, decreasing the subjects' Evaluating Respiratory Symptoms in COPD Score (E-RS), including the subjects' Evaluating Respiratory Symptoms in COPD Score (E-RS), compared to the subjects' Evaluating Respiratory Symptoms in COPD Score (E-RS) prior to treatment. In some embodiments, treating the respiratory enterovirus infection in the subject includes preventing an increase in the subjects' Evaluating Respiratory Symptoms in COPD Score (E-RS) compared to the subjects' Evaluating Respiratory Symptoms in COPD Score (E-RS) score prior to treatment.

In some embodiments, treating the respiratory enterovirus infection in the subject includes decreasing the subjects' peak lower respiratory symptom score (LRSS), such as peak lower respiratory symptom score (LRSS), compared to the subjects' peak lower respiratory symptom score (LRSS) prior to treatment. Similarly, treating the respiratory enterovirus infection in the subject may include preventing an increase in the subjects' LRSS compared to the subjects' LRSS prior to treatment. In some embodiments, treating the respiratory enterovirus infection in the subject includes decreasing and/or preventing an increase in the subjects' peak upper respiratory symptom score (URSS), such as URSS, compared to the subjects' peak lower respiratory symptom score (URSS) prior to treatment.

In some embodiments, treating the respiratory enterovirus infection in the subject includes decreasing the subjects' EXAcerbation of Chronic Pulmonary Disease Tool—Subject Reported Outcome (EXACT-PRO), such as the subjects' EXAcerbation of Chronic Pulmonary Disease Tool—Subject Reported Outcome (EXACT-PRO) score, compared to the subjects' EXAcerbation of Chronic Pulmonary Disease Tool—Subject Reported Outcome (EXACT-PRO) prior to treatment. Similarly, treating the respiratory enterovirus infection in the subject may include preventing an increase in the subjects' EXACT-PRO, such as the subjects' EXACT-PRO score.

In some embodiments, treating the respiratory enterovirus infection in the subject includes decreasing the subjects' EXAcerbation of Chronic Pulmonary Disease Tool—Subject Respiratory Symptoms (EXACT-RS), such as EXAcerbation of Chronic Pulmonary Disease Tool—Subject Respiratory Symptoms (EXACT-RS) score, compared to the subjects' EXAcerbation of Chronic Pulmonary Disease Tool—Subject Respiratory Symptoms (EXACT-RS) prior to treatment. In some embodiments, treating the respiratory enterovirus infection in the subject includes preventing an increase in the subjects' EXACT-RS score.

In some embodiments, treating the respiratory enterovirus infection in the subject includes decreasing and/or preventing an increase in the subjects' COPD assessment tool (CAT) score compared to the subjects' COPD assessment tool (CAT) score prior to treatment.

In some embodiments, treating the respiratory enterovirus infection in the subject includes decreasing the subjects' St. George's Respiratory Questionnaire (SGRQ) score compared to the subjects' St. George's Respiratory Questionnaire (SGRQ) score prior to treatment. In some embodiments, treating the respiratory enterovirus infection in the subject includes preventing an increase in the subjects' St. George's Respiratory Questionnaire (SGRQ) score compared to the subjects' St. George's Respiratory Questionnaire (SGRQ) score prior to treatment.

In some embodiments, treating the respiratory enterovirus infection in the subject includes decreasing and/or preventing an increase in the subjects' Wisconsin Upper Respiratory Symptom Survey (WURSS) score compared to the subjects' Wisconsin Upper Respiratory Symptom Survey (WURSS) score prior to treatment.

In some embodiments, treating the respiratory enterovirus in the subject includes decreasing the subject's Asthma Control Questionnaire (ACQ) score compared to the subject's ACQ score prior to treatment. In some embodiments, treating the respiratory enterovirus in the subject includes preventing an increase in the subject's Asthma Control Questionnaire (ACQ) score compared to the subject's ACQ score prior to treatment. Different versions of the ACQ, including but not limited to versions 5, 6, or 7, may be used.

In some embodiments, treating the respiratory enterovirus in the subject includes decreasing the subject's Asthma Control Test (ACT) score compared to the subject's ACT score prior to treatment. In some embodiments, treating the respiratory enterovirus in the subject includes preventing an increase in the subject's Asthma Control Test (ACT) score compared to the subject's ACT score prior to treatment.

In some embodiments, treating the respiratory enterovirus in the subject includes decreasing the subject's Pediatric Asthma Symptom Scale (PASS) score compared to the subject's PASS score prior to treatment. In some embodiments, treating the respiratory enterovirus in the subject includes preventing an increase in the subject's Pediatric Asthma Symptom Scale (PASS) score compared to the subject's PASS score prior to treatment.

In some embodiments, treating the respiratory enterovirus in the subject includes decreasing the subject's Asthma Severity Scoring System (ASSESS) score compared to the subject's ASSESS score prior to treatment. In some embodiments, treating the respiratory enterovirus in the subject includes preventing an increase in the subject's Pediatric Asthma Severity Scoring System (ASSESS) score compared to the subject's ASSESS score prior to treatment.

In some embodiments, treating the respiratory enterovirus in the subject includes decreasing the subject's Pediatric Asthma Score (PAS) score compared to the subject's PAS score prior to treatment. In some embodiments, treating the respiratory enterovirus in the subject includes preventing an increase in the subject's Pediatric Asthma Score (PAS) score compared to the subject's PAS score prior to treatment.

In some embodiments, treating the respiratory enterovirus in the subject includes decreasing the subject's Pediatric Respiratory Assessment Measure (PRAM) score compared to the subject's PRAM score prior to treatment. In some embodiments, treating the respiratory enterovirus in the subject includes preventing an increase in the subject's Pediatric Respiratory Assessment Measure (PRAM) compared to the subject's PRAM score prior to treatment.

In some embodiments, treating the respiratory enterovirus in the subject includes decreasing the subject's Pediatric Asthma Risk Score (PARS) score compared to the subject's PARS score prior to treatment. In some embodiments, treating the respiratory enterovirus in the subject includes preventing an increase in the subject's Pediatric Asthma Risk Score (PARS) compared to the subject's PARS score prior to treatment.

In some embodiments, treating the respiratory enterovirus infection in the subject includes improving the subject's lung function compared to the subjects' lung function prior to treatment. The subjects' lung function may, in some embodiments, be measured by methods familiar to those skilled in the art, including Forced Expiratory Volume −1 (FEV₋₁; in liters and % predicted), Forced Vital Capacity (FVC; in liters and % predicted) FEV₁/FVC ratio and peak expiratory flow (PEF), or any combination thereof.

In some embodiments, treating the respiratory enterovirus infection in the subject includes preventing a decrease in the subjects' lung function compared to the subjects' lung function prior to treatment, wherein the subjects' lung function is measured by Forced Expiratory Volume −1 (FEV₋₁; in liters and % predicted), Forced Vital Capacity (FVC; in liters and % predicted) FEV₁/FVC ratio and peak expiratory flow (PEF), or any combination thereof.

In some embodiments, treating the respiratory enterovirus infection in the subject includes reducing the subjects' peak nasal lavage viral load compared to the subjects' peak nasal lavage viral load prior to treatment. In some embodiments, treating the respiratory enterovirus infection in the subject includes preventing an increase in the subjects' peak nasal lavage viral load compared to the subjects' peak nasal lavage viral load prior to treatment.

In some embodiments, treating the respiratory enterovirus infection in the subject includes reducing and/or preventing an increase in the subjects' peak sputum lavage viral load compared to the subjects' peak sputum lavage viral load prior to treatment. In some embodiments, treating the respiratory enterovirus infection in the subject includes reducing and/or preventing bacterial bronchitis, pneumonia, or combinations thereof.

In some embodiments, treating the respiratory enterovirus infection in the subject includes reducing the subjects' AUC nasal viral load compared to the subjects' AUC nasal viral load prior to treatment, and in some embodiments, treating the respiratory enterovirus infection in the subject includes preventing an increase in the subjects' AUC nasal viral load compared to the subjects' AUC nasal viral load prior to treatment. Treating the respiratory enterovirus infection in the subject may, in some embodiments, include reducing and/or preventing an increase in the subjects' AUC sputum viral load compared to the subjects' AUC sputum viral load prior to treatment.

In some embodiments, treating the respiratory enterovirus infection in the subject includes reducing the duration of viral shedding, reducing the number of days wherein the subject is positive for bacteria in the sputum, or combinations thereof.

In some embodiments, treating the respiratory enterovirus infection in the subject includes reducing and/or preventing an increase in the subjects' peak sputum bacterial load compared to the subjects' peak sputum lavage bacterial load prior to treatment. In some embodiments, treating the respiratory enterovirus infection in the subject includes reducing and/or preventing bacterial bronchitis, pneumonia, or combinations thereof. In some embodiments, treating the respiratory enterovirus infection in the subject includes reducing and/or preventing an increase in the subjects' AUC sputum bacterial load compared to the subjects' AUC sputum bacterial load prior to treatment.

In some embodiments, treating the respiratory enterovirus infection in the subject includes preventing, or reducing, acute exacerbations of COPD in the subject caused by the respiratory enterovirus infection. Treating the respiratory enterovirus infection in the subject may in some embodiments include a reduction in the frequency of acute COPD exacerbations in the subject, a reduction in the severity of acute COPD exacerbations in the subject, a reduction in the duration of acute COPD exacerbations in the subject or combinations thereof, caused by the respiratory enterovirus infection. In some embodiments, treating the respiratory enterovirus infection in the subject includes preventing an increase in the frequency of acute COPD exacerbations in the subject, an increase in the severity of acute COPD exacerbations in the subject, an increase in the duration of acute COPD exacerbations in the subject or combinations thereof, caused by the respiratory enterovirus infection. In some embodiments, treating the respiratory condition in the subject as described herein may eliminate the need for a chronic immune therapy to be administered to the subject. In some embodiments, elimination of the need for chronic immune therapy is due to the compositions and methods of the present disclosure minimizing a Type 2 immune response phenotype in the subject. In some embodiments, elimination of the need for chronic immune therapy is due to direct anti-viral treatment of the exacerbation trigger, wherein the exacerbation trigger may be a respiratory enterovirus.

In some embodiments, administering to the subject a therapeutically effective amount of vapendavir sulfate exceeds the paEC₅₀ for human rhinoviruses. In some embodiments, the paEC₅₀ for human rhinoviruses is about 75.4 ng/mL. In some embodiments, the therapeutically effective amount of vapendavir sulfate is a single loading dose of about 1,000 mg followed by a maintenance dose of about 500 mg every 12 hours for about 4 to about 10 days. In some embodiments, the therapeutically effective amount of vapendavir sulfate is a single loading dose of about 1,000 mg followed a maintenance dose of about 500 mg every 12 hours for a total of about 13 maintenance doses. In some embodiments, the therapeutically effective amount of vapendavir sulfate is an amount that achieves a Cmax of about 2000 to about 12000 ng/mL. In some embodiments, the paEC₅₀ for human rhinoviruses is exceeded for about 4 to about 10 days or until treatment is stopped.

In some embodiments, administering to the subject a therapeutically effective amount of vapendavir sulfate exceeds the paEC₉₀ for human rhinoviruses. In some embodiments, the paEC₉₀ for human rhinoviruses is about 678.6 ng/mL. In some embodiments, the therapeutically effective amount of vapendavir sulfate is a single loading dose of about 1,000 mg followed by a maintenance dose of about 500 mg every 12 hours for about 4 to about 10 days. In some embodiments, the therapeutically effective amount of vapendavir sulfate is a single loading dose of about 1,000 mg followed a maintenance dose of about 500 mg every 12 hours for a total of about 13 maintenance doses. In some embodiments, the therapeutically effective amount of vapendavir sulfate is an amount that achieves a Cmax of about 2000 to about 12000 ng/mL. In some embodiments, the paEC₉₀ for human rhinoviruses is exceeded for about 4 to about 10 days or until treatment is stopped.

In some embodiments, administering to the subject a therapeutically effective amount of vapendavir sulfate results in a Cₘₐₓ that exceeds the paEC₅₀ for human rhinoviruses. In some embodiments, the paEC₅₀ for human rhinoviruses is about 75.4 ng/mL. In some embodiments, the therapeutically effective amount of vapendavir sulfate is a single loading dose of about 1,000 mg followed by a maintenance dose of about 500 mg every 12 hours for about 4 to about 10 days. In some embodiments, the therapeutically effective amount of vapendavir sulfate is a single loading dose of about 1,000 mg followed a maintenance dose of about 500 mg every 12 hours for a total of about 13 maintenance doses. In some embodiments, the therapeutically effective amount of vapendavir sulfate is an amount that achieves a Cmax of about 2000 to about 12000 ng/mL. In some embodiments, the paEC₅₀ for human rhinoviruses is exceeded for about 4 to about 10 days or until treatment is stopped.

In some embodiments, administering to the subject a therapeutically effective amount of vapendavir sulfate results in a Cₘₐₓ that exceeds the paEC₉₀ for human rhinoviruses. In some embodiments, the paEC₉₀ for human rhinoviruses is about 678.6 ng/mL. In some embodiments, the therapeutically effective amount of vapendavir sulfate is a single loading dose of about 1,000 mg followed by a maintenance dose of about 500 mg every 12 hours for about 4 to about 10 days. In some embodiments, the therapeutically effective amount of vapendavir sulfate is a single loading dose of about 1,000 mg followed a maintenance dose of about 500 mg every 12 hours for a total of about 13 maintenance doses. In some embodiments, the therapeutically effective amount of vapendavir sulfate is an amount that achieves a Cmax of about 2000 to about 12000 ng/mL. In some embodiments, the paEC₉₀ for human rhinoviruses is exceeded for about 4 to about 10 days or until treatment is stopped.

In some embodiments, administering to the subject a therapeutically effective amount of vapendavir sulfate exceeds the paEC₅₀ for human rhinoviruses following a single dose. In some embodiments, the paEC₅₀ for human rhinoviruses is about 75.4 ng/mL. In some embodiments, the therapeutically effective amount of vapendavir sulfate is a single loading dose of about 1,000 mg followed by a maintenance dose of about 500 mg every 12 hours for about 4 to about 10 days. In some embodiments, the therapeutically effective amount of vapendavir sulfate is a single loading dose of about 1,000 mg followed a maintenance dose of about 500 mg every 12 hours for a total of about 13 maintenance doses. In some embodiments, the therapeutically effective amount of vapendavir sulfate is an amount that achieves a Cmax of about 2000 to about 12000 ng/mL. In some embodiments, the paEC₅₀ for human rhinoviruses is exceeded for about 4 to about 10 days or until treatment is stopped.

In some embodiments, administering to the subject a therapeutically effective amount of vapendavir sulfate exceeds the paEC₉₀ for human rhinoviruses following a single dose. In some embodiments, the paEC₉₀ for human rhinoviruses is about 678.6 ng/mL. In some embodiments, the therapeutically effective amount of vapendavir sulfate is a single loading dose of about 1,000 mg followed by a maintenance dose of about 500 mg every 12 hours for about 4 to about 10 days. In some embodiments, the therapeutically effective amount of vapendavir sulfate is a single loading dose of about 1,000 mg followed a maintenance dose of about 500 mg every 12 hours for a total of about 13 maintenance doses. In some embodiments, the therapeutically effective amount of vapendavir sulfate is an amount that achieves a Cmax of about 2000 to about 12000 ng/mL. In some embodiments, the paEC₉₀ for human rhinoviruses is exceeded for about 4 to about 10 days or until treatment is stopped.

In some embodiments, administering to the subject a therapeutically effective amount of vapendavir sulfate results in the subject having a plasma concentration above paEC₉₀ and paEC₅₀ after 1 day of treatment, after 2 days of treatment, after 5 days of treatment, or a combination thereof.

In some embodiments, the method includes administering to the subject a solid oral dosage composition which includes a therapeutically effective amount of vapendavir sulfate, wherein the vapendavir sulfate may be in a crystalline form. The vapendavir sulfate which is included in the solid oral dosage composition may be in any of the crystalline forms or combinations thereof as disclosed herein. In some embodiments, the method includes administering vapendavir sulfate which is in a crystalline form having an X-ray powder diffraction (XRPD) pattern which exhibits peaks at diffraction angles of 4.0°, 8.0°, 15.9°, 20.0°, 21.0°, and 28.1° (2θ±0.2°).

In some embodiments, there is provided a of treating a subject in need thereof, the method including administering to the subject a solid oral dosage composition, such as a solid oral composition including a therapeutically effective amount, such as about 250 mg to about 2000 mg, of Crystalline Form A of vapendavir sulfate, and one or more pharmaceutically acceptable excipients, such as a diluent, a disintegrant, a surfactant, a lubricant, a color coating, or any combination thereof. In some embodiments, the subject has a respiratory condition as described herein.

In some embodiments, there is provided a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base every about 12 hours thereafter for about 12 to about 20 maintenance doses, wherein the subject is in a fasted state when orally administering the loading dose and the maintenance doses, wherein the respiratory enterovirus in the subject is treated. In some embodiments, orally administering the loading dose to the subject results in a Cₘₐₓ of about 2400 ng/mL to about 3750 ng/mL of vapendavir, such as about 2400 ng/mL, about 2600 ng/mL, about 2800 ng/mL, about 3000 ng/mL, about 3200 ng/mL, about 3400 ng/mL, about 3600 ng/mL, about 3750 ng/mL, or any value contained within a range formed by any two of the preceding values. In some embodiments, orally administering the loading dose to the subject results in the Cₘₐₓ value being reached in a time of about 2 hours, that, is a Tₘₐₓ value may be about 2 hours. In some embodiments, the subject has COPD. In some embodiments, the subject has asthma. In some embodiments, the subject is not taking an acid reducer when orally administering the loading dose.

In some embodiments, there is provided a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base every about 12 hours thereafter for about 12 to about 20 maintenance doses, wherein the subject is in a fed state when orally administering the loading dose and the maintenance doses, wherein the respiratory enterovirus in the subject is treated. In some embodiments, orally administering the loading dose to the subject results in a Cₘₐₓ of about 6400 ng/mL to about 10000 ng/mL of vapendavir, such as about 6400 ng/mL, about 7000 ng/mL, about 7500 ng/mL, about 8000 ng/mL, about 8500 ng/mL, about 9000 ng/mL, about 9500 ng/mL, about 10000 ng/mL, or any value contained within a range formed by any two of the preceding values. In some embodiments, orally administering the loading dose to the subject results in the Cₘₐₓ value being reached in a time of about 4 hours, that, is a Tₘₐₓ value may be about 4 hours. In some embodiments, the subject has COPD. In some embodiments, the subject has asthma. In some embodiments, the subject is not taking an acid reducer when orally administering the loading dose.

In some embodiments, there is provided a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base every about 12 hours thereafter for about 12 to about 20 maintenance doses, wherein the subject is in a fed state when orally administering the loading dose and the maintenance doses, and wherein the subject is taking an acid reducer when orally administering the loading dose and the maintenance doses, wherein the respiratory enterovirus in the subject is treated. In some embodiments, orally administering the loading dose to the subject results in a Cₘₐₓ of about 2400 ng/mL to about 3750 ng/mL of vapendavir, such as about 2400 ng/mL, about 2600 ng/mL, about 2800 ng/mL, about 3000 ng/mL, about 3200 ng/mL, about 3400 ng/mL, about 3600 ng/mL, about 3750 ng/mL, or any value contained within a range formed by any two of the preceding values. In some embodiments, orally administering the loading dose to the subject results in the Cₘₐₓ value being reached in a time of about 6 hours, that, is a Tₘₐₓ value may be about 6 hours. In some embodiments, the subject has COPD. In some embodiments, the subject has asthma.

In some embodiments, there is provided a method of treating a respiratory enterovirus in a subject in need thereof, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base every about 12 hours thereafter for about 12 to about 20 maintenance doses, wherein the subject is in a fasted state when orally administering the loading dose and the maintenance doses, and wherein the subject is taking an acid reducer when orally administering the loading dose and the maintenance doses, wherein the respiratory enterovirus in the subject is treated. In some embodiments, orally administering the loading dose to the subject results in a Cₘₐₓ of about 1600 ng/mL to about 2500 ng/mL of vapendavir, such as about 1600 ng/mL, about 1700 ng/mL, about 1800 ng/mL, about 1900 ng/mL, about 2000 ng/mL, about 2100 ng/mL, about 2200 ng/mL, about 2300 ng/mL, about 2400 ng/mL, about 2500 ng/mL, or any value contained within a range formed by any two of the preceding values. In some embodiments, orally administering the loading dose to the subject results in the Cₘₐₓ value being reached in a time of about 2 hours, that, is a Tₘₐₓ value may be about 2 hours. In some embodiments, the subject has COPD. In some embodiments, the subject has asthma.

The ranges for Cₘₐₓ and other pharmacokinetic parameters disclosed herein are intended to reflect the ranges for bioequivalence set by the Food and Drug Administration (FDA). It is generally understood that to establish bioequivalence of a particular drug, such as a generic, the calculated confidence interval should typically fall within 80-125% of the referenced drug. Further details on acceptable ranges for bioequivalence can be found in “Guidance for Industry: Statistical Approaches to Establishing Bioequivalence”, January 2001, from U.S. Department of Health and Human Services, Food and Drug Administration, Center for Drug Evaluation and Research (CDER), which is incorporated by reference herein in its entirety. The December 2022 “Statistical Approaches to Establishing Bioequivalence” Draft Guidance, which is also incorporated by reference herein in its entirety, may also be consulted.

In some embodiments, there is provided a method of treating a common cold in a human subject, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base every about 12 hours thereafter for about 12 to about 20 maintenance doses. In some embodiments, the human subject is otherwise healthy; that is, the human subject does not have any preexisting conditions.

In some embodiments, there is provided a method of treating a rhinovirus infection in a human subject, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base every about 12 hours thereafter for about 12 to about 20 maintenance doses, wherein treating decreases the risk of a rhinovirus infection in the human subject. The pharmaceutical compositions and methods disclosed herein may be used as a prophylactic in a human subject. In some embodiments, the human subject is considered at-risk; for example, the human subject may be elderly, have asthma, COPD, or other lung disease, or any combination thereof.

In some embodiments, there is provided a method of treating a rhinovirus infection, an enterovirus infection, a respiratory syncytial virus infection, or any combination thereof in a human subject, including orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base every about 12 hours thereafter for about 12 to about 20 maintenance doses. In some embodiments, the human subject is a child. In some embodiments, treating the rhinovirus infection, the enterovirus infection, the respiratory syncytial virus infection, or any combination thereof in the human subject prevents bronchiolitis, sepsis, meningitis, polio-like paralysis, or any combination thereof in the human subject.

The methods and compositions of the present disclosure may be employed in the following embodiments, which may be combined as appropriate to form new embodiments.

The present disclosure includes but is not limited to the following embodiments.
1. A pharmaceutical composition comprising a therapeutically effective amount of vapendavir sulfate and one or more pharmaceutically acceptable excipients,
   wherein the vapendavir sulfate is in the form of Crystalline Form A,
   wherein the vapendavir sulfate has a particle size distribution with a D90 of about 35 μm to about 140 μm, a D50 of about 10 μm to about 25 μm, and a D10 of about 2.5 μm to about 10 μm, and
   wherein the pharmaceutical composition is a solid oral dosage form.
2. The pharmaceutical composition of embodiment 1, wherein the therapeutically effective amount of vapendavir sulfate is about 100 mg to about 1000 mg.
3. The pharmaceutical composition of embodiment 1, wherein the therapeutically effective amount of vapendavir sulfate is about 250 mg to about 800 mg.
4. The pharmaceutical composition of embodiment 1, wherein the therapeutically effective amount of vapendavir sulfate is about 500 mg.
5. The pharmaceutical composition of embodiment 1, wherein the vapendavir sulfate has a polymorphic purity of at least about 90%.
6. The pharmaceutical composition of embodiment 1, wherein the vapendavir sulfate has a particle size distribution with a D90 of about 100 μm, a D50 of about 25 μm, and a D10 of about 10 μm.
7. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days,
   wherein orally administering the loading dose to the subject results in a Cₘₐₓ of about 2400 ng/mL to about 3750 ng/mL of vapendavir; and
   wherein the subject is in a fasted state when orally administering the loading dose and the maintenance doses;
   wherein the respiratory enterovirus in the subject is treated.
8. The method of embodiment 7, wherein orally administering the loading dose to the subject results in a Cₘₐₓ of about 3000 ng/mL.
9. The method of embodiment 7, wherein the subject has COPD.
10. The method of embodiment 7, wherein the subject has asthma.
11. The method of embodiment 7, wherein a Tₘₐₓ is about 2 hours.
12. The method of embodiment 7, wherein the subject is not taking an acid reducer when orally administering the loading dose and the maintenance doses.
13. The method of embodiment 7, wherein the loading dose is administered within about 1 to about 7 days of the subject displaying symptoms of the respiratory enterovirus.
14. The method of embodiment 7, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.
15. The method of embodiment 7, wherein the subject has COPD, and wherein treating the respiratory condition in the subject comprises decreasing a Evaluating Respiratory Symptoms in COPD Score (E-RS) in the subject compared to a Evaluating Respiratory Symptoms in COPD Score (E-RS) in the subject prior to treatment.
16. The method of embodiment 7, wherein the subject has COPD, and wherein treating the respiratory condition in the subject comprises preventing an increase in a Evaluating Respiratory Symptoms in COPD Score (E-RS) in the subject compared to a Evaluating Respiratory Symptoms in COPD Score (E-RS) in the subject prior to treatment.
17. The method of embodiment 7, wherein the subject has COPD, and wherein treating the respiratory condition in the subject comprises decreasing a peak lower respiratory symptom score (LRSS) in the subject compared to a peak lower respiratory symptom score (LRSS) in the subject prior to treatment.
18. The method of embodiment 7, wherein the subject has COPD, and wherein treating the respiratory condition in the subject comprises preventing an increase in a peak lower respiratory symptom score (LRSS) in the subject compared to a peak lower respiratory symptom score (LRSS) in the subject prior to treatment.
19. The method of embodiment 7, wherein the subject has COPD, and wherein treating the respiratory condition in the subject comprises decreasing a peak upper respiratory symptom score (URSS) in the subject compared to a peak upper respiratory symptom score (URSS) in the subject prior to treatment.
20. The method of embodiment 7, wherein the subject has COPD, and wherein treating the respiratory condition in the subject comprises preventing an increase in a peak upper respiratory symptom score (URSS) in the subject compared to a peak upper respiratory symptom score (URSS) in the subject prior to treatment.
21. The method of embodiment 7, wherein the subject has COPD, and wherein treating the respiratory condition in the subject comprises decreasing or preventing an increase in an EXAcerbation of Chronic Pulmonary Disease Tool—Patient Reported Outcome (EXACT-PRO) in the subject compared to an EXAcerbation of Chronic Pulmonary Disease Tool—Patient Reported Outcome (EXACT-PRO) in the subject prior to treatment.
22. The method of embodiment 7, wherein the subject has COPD, and wherein treating the respiratory condition in the subject comprises decreasing or preventing an increase in an EXAcerbation of Chronic Pulmonary Disease Tool—Patient Respiratory Symptoms (EXACT-RS) in the subject compared to an EXAcerbation of Chronic Pulmonary Disease Tool—Patient Respiratory Symptoms (EXACT-RS) in the subject prior to treatment.
23. The method of embodiment 7, wherein the subject has asthma, and wherein treating the respiratory enterovirus in the subject comprises decreasing the subject's Asthma Control Questionnaire (ACQ) score compared to the subject's ACQ score prior to treatment.
24. The method of embodiment 7, wherein the subject has asthma, and wherein treating the respiratory enterovirus in the subject comprises preventing an increase in the subject's Asthma Control Questionnaire (ACQ) score compared to the subject's ACQ score prior to treatment.
25. The method of embodiment 7, wherein the subject has asthma, and wherein treating the respiratory enterovirus in the subject comprises decreasing the subject's Asthma Control Test (ACT) score compared to the subject's ACT score prior to treatment.
26. The method of embodiment 7, wherein the subject has asthma, and wherein treating the respiratory enterovirus in the subject comprises preventing an increase in the subject's Asthma Control Test (ACT) score compared to the subject's ACT score prior to treatment.
27. The method of embodiment 7, wherein the subject has asthma, and wherein treating the respiratory enterovirus in the subject comprises decreasing the subject's Pediatric Asthma Symptom Scale (PASS) score compared to the subject's PASS score prior to treatment.
28. The method of embodiment 7, wherein the subject has asthma, and wherein treating the respiratory enterovirus in the subject comprises preventing an increase in the subject's Pediatric Asthma Symptom Scale (PASS) score compared to the subject's PASS score prior to treatment.
29. The method of embodiment 7, wherein the subject has asthma, and wherein treating the respiratory enterovirus in the subject comprises decreasing the subject's Asthma Severity Scoring System (ASSESS) score compared to the subject's ASSESS score prior to treatment.
30. The method of embodiment 7, wherein the subject has asthma, and wherein treating the respiratory enterovirus in the subject comprises preventing an increase in the subject's Asthma Severity Scoring System (ASSESS) score compared to the subject's ASSESS score prior to treatment.
31. The method of embodiment 7, wherein the subject has asthma, and wherein treating the respiratory enterovirus in the subject comprises decreasing the subject's Pediatric Asthma Score (PAS) score compared to the subject's PAS score prior to treatment.
32. The method of embodiment 7, wherein the subject has asthma, and wherein treating the respiratory enterovirus in the subject comprises preventing an increase in the subject's Pediatric Asthma Score (PAS) score compared to the subject's PAS score prior to treatment.
33. The method of embodiment 7, wherein the subject has asthma, and wherein treating the respiratory enterovirus in the subject comprises decreasing the subject's Pediatric Respiratory Assessment Measure (PRAM) score compared to the subject's PRAM score prior to treatment.
34. The method of embodiment 7, wherein the subject has asthma, and wherein treating the respiratory enterovirus in the subject comprises preventing an increase in the subject's Pediatric Respiratory Assessment Measure (PRAM) score compared to the subject's PRAM score prior to treatment.
35. The method of embodiment 7, wherein the subject has asthma, and wherein treating the respiratory enterovirus in the subject comprises decreasing the subject's Pediatric Asthma Risk Score (PARS) score compared to the subject's PARS score prior to treatment.
36. The method of embodiment 7, wherein the subject has asthma, and wherein treating the respiratory enterovirus in the subject comprises preventing an increase in the subject's Pediatric Asthma Risk Score (PARS) score compared to the subject's PARS score prior to treatment.
37. The method of embodiment 7, wherein treating the respiratory enterovirus in the subject comprises preventing a decrease in the subject's lung function compared to the subject's lung function prior to treatment.
38. The method of embodiment 37, wherein the subject's lung function is measured by Forced Expiratory Volume −1 (FEV₋₁; in liters and % predicted), Forced Vital Capacity (FVC; in liters and % predicted) FEV₁/FVC ratio and peak expiratory flow (PEF) or any combination thereof.
39. The method of embodiment 7, wherein treating the respiratory enterovirus in the subject comprises reducing or preventing an increase in the subject's peak nasal lavage viral load compared to the subject's peak nasal lavage viral load prior to treatment.
40. The method of embodiment 7, wherein treating the respiratory enterovirus in the subject comprises reducing or preventing an increase in the subject's peak sputum lavage viral load compared to the subject's peak sputum lavage viral load prior to treatment.
41. The method of embodiment 7, wherein treating the respiratory enterovirus in the subject comprises reducing or preventing an increase in the subject's AUC nasal viral load compared to the subject's AUC nasal viral load prior to treatment.
42. The method of embodiment 7, wherein treating the respiratory enterovirus in the subject comprises reducing or preventing an increase in the subject's AUC sputum viral load compared to the subject's AUC sputum viral load prior to treatment.
43. The method of embodiment 7, wherein treating the respiratory enterovirus in the subject comprises reducing the duration of viral shedding.
44. The method of embodiment 7, wherein treating the respiratory enterovirus in the subject comprises reducing or preventing an increase in the subject's peak sputum bacterial load compared to the subject's peak sputum lavage bacterial load prior to treatment.
45. The method of embodiment 7, wherein treating the respiratory enterovirus in the subject comprises reducing or preventing an increase in the subject's AUC sputum bacterial load compared to the subject's AUC sputum bacterial load prior to treatment.
46. The method of embodiment 7, wherein treating the respiratory enterovirus in the subject comprises reducing the number of days wherein the subject is positive for bacteria in the sputum.
47. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days,
   wherein orally administering the loading dose to the subject results in a Cₘₐₓ of about 6400 ng/mL to about 10000 ng/mL of vapendavir; and
   wherein the subject is in a fed state when orally administering the loading dose and the maintenance doses;
   wherein the respiratory enterovirus in the subject is treated.
48. The method of embodiment 47, wherein orally administering the loading dose to the subject results in a Cₘₐₓ of about 8000 ng/mL.
49. The method of embodiment 47, wherein the subject has COPD.
50. The method of embodiment 47, wherein the subject has asthma.
51. The method of embodiment 47, wherein a Tₘₐₓ is about 4 hours.
52. The method of embodiment 47, wherein the subject is not taking an acid reducer when orally administering the loading dose and the maintenance doses.
53. The method of embodiment 47, wherein the loading dose is administered within about 1 to about 7 days of the subject displaying symptoms of the respiratory enterovirus.
54. The method of embodiment 47, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.
55. The method of embodiment 47, wherein the subject has COPD, and wherein treating the respiratory condition in the subject comprises decreasing a Evaluating Respiratory Symptoms in COPD Score (E-RS) in the subject compared to a Evaluating Respiratory Symptoms in COPD Score (E-RS) in the subject prior to treatment.
56. The method of embodiment 47, wherein the subject has COPD, and wherein treating the respiratory condition in the subject comprises preventing an increase in a Evaluating Respiratory Symptoms in COPD Score (E-RS) in the subject compared to a Evaluating Respiratory Symptoms in COPD Score (E-RS) in the subject prior to treatment.
57. The method of embodiment 47, wherein the subject has COPD, and wherein treating the respiratory condition in the subject comprises decreasing a peak lower respiratory symptom score (LRSS) in the subject compared to a peak lower respiratory symptom score (LRSS) in the subject prior to treatment.
58. The method of embodiment 47, wherein the subject has COPD, and wherein treating the respiratory condition in the subject comprises preventing an increase in a peak lower respiratory symptom score (LRSS) in the subject compared to a peak lower respiratory symptom score (LRSS) in the subject prior to treatment.
59. The method of embodiment 47, wherein the subject has COPD, and wherein treating the respiratory condition in the subject comprises decreasing a peak upper respiratory symptom score (URSS) in the subject compared to a peak upper respiratory symptom score (URSS) in the subject prior to treatment.
60. The method of embodiment 47, wherein the subject has COPD, and wherein treating the respiratory condition in the subject comprises preventing an increase in a peak upper respiratory symptom score (URSS) in the subject compared to a peak upper respiratory symptom score (URSS) in the subject prior to treatment.
61. The method of embodiment 47, wherein the subject has COPD, and wherein treating the respiratory condition in the subject comprises decreasing or preventing an increase in an EXAcerbation of Chronic Pulmonary Disease Tool—Patient Reported Outcome (EXACT-PRO) in the subject compared to an EXAcerbation of Chronic Pulmonary Disease Tool—Patient Reported Outcome (EXACT-PRO) in the subject prior to treatment.
62. The method of embodiment 47, wherein the subject has COPD, and wherein treating the respiratory condition in the subject comprises decreasing or preventing an increase in an EXAcerbation of Chronic Pulmonary Disease Tool—Patient Respiratory Symptoms (EXACT-RS) in the subject compared to an EXAcerbation of Chronic Pulmonary Disease Tool—Patient Respiratory Symptoms (EXACT-RS) in the subject prior to treatment.
63. The method of embodiment 47, wherein the subject has asthma, and wherein treating the respiratory enterovirus in the subject comprises decreasing the subject's Asthma Control Questionnaire (ACQ) score compared to the subject's ACQ score prior to treatment.
64. The method of embodiment 47, wherein the subject has asthma, and wherein treating the respiratory enterovirus in the subject comprises preventing an increase in the subject's Asthma Control Questionnaire (ACQ) score compared to the subject's ACQ score prior to treatment.
65. The method of embodiment 47, wherein the subject has asthma, and wherein treating the respiratory enterovirus in the subject comprises decreasing the subject's Asthma Control Test (ACT) score compared to the subject's ACT score prior to treatment.
66. The method of embodiment 47, wherein the subject has asthma, and wherein treating the respiratory enterovirus in the subject comprises preventing an increase in the subject's Asthma Control Test (ACT) score compared to the subject's ACT score prior to treatment.
67. The method of embodiment 47, wherein the subject has asthma, and wherein treating the respiratory enterovirus in the subject comprises decreasing the subject's Pediatric Asthma Symptom Scale (PASS) score compared to the subject's PASS score prior to treatment.
68. The method of embodiment 47, wherein the subject has asthma, and wherein treating the respiratory enterovirus in the subject comprises preventing an increase in the subject's Pediatric Asthma Symptom Scale (PASS) score compared to the subject's PASS score prior to treatment.
69. The method of embodiment 47, wherein the subject has asthma, and wherein treating the respiratory enterovirus in the subject comprises decreasing the subject's Asthma Severity Scoring System (ASSESS) score compared to the subject's ASSESS score prior to treatment.
70. The method of embodiment 47, wherein the subject has asthma, and wherein treating the respiratory enterovirus in the subject comprises preventing an increase in the subject's Asthma Severity Scoring System (ASSESS) score compared to the subject's ASSESS score prior to treatment.
71. The method of embodiment 47, wherein the subject has asthma, and wherein treating the respiratory enterovirus in the subject comprises decreasing the subject's Pediatric Asthma Score (PAS) score compared to the subject's PAS score prior to treatment.
72. The method of embodiment 47, wherein the subject has asthma, and wherein treating the respiratory enterovirus in the subject comprises preventing an increase in the subject's Pediatric Asthma Score (PAS) score compared to the subject's PAS score prior to treatment.
73. The method of embodiment 47, wherein the subject has asthma, and wherein treating the respiratory enterovirus in the subject comprises decreasing the subject's Pediatric Respiratory Assessment Measure (PRAM) score compared to the subject's PRAM score prior to treatment.
74. The method of embodiment 47, wherein the subject has asthma, and wherein treating the respiratory enterovirus in the subject comprises preventing an increase in the subject's Pediatric Respiratory Assessment Measure (PRAM) score compared to the subject's PRAM score prior to treatment.
75. The method of embodiment 47, wherein the subject has asthma, and wherein treating the respiratory enterovirus in the subject comprises decreasing the subject's Pediatric Asthma Risk Score (PARS) score compared to the subject's PARS score prior to treatment.
76. The method of embodiment 47, wherein the subject has asthma, and wherein treating the respiratory enterovirus in the subject comprises preventing an increase in the subject's Pediatric Asthma Risk Score (PARS) score compared to the subject's PARS score prior to treatment.
77. The method of embodiment 47, wherein treating the respiratory enterovirus in the subject comprises preventing a decrease in the subject's lung function compared to the subject's lung function prior to treatment.
78. The method of embodiment 77, wherein the subject's lung function is measured by Forced Expiratory Volume −1 (FEV₋₁; in liters and % predicted), Forced Vital Capacity (FVC; in liters and % predicted) FEV₁/FVC ratio and peak expiratory flow (PEF) or any combination thereof.
79. The method of embodiment 47, wherein treating the respiratory enterovirus in the subject comprises reducing or preventing an increase in the subject's peak nasal lavage viral load compared to the subject's peak nasal lavage viral load prior to treatment.
80. The method of embodiment 47, wherein treating the respiratory enterovirus in the subject comprises reducing or preventing an increase in the subject's peak sputum lavage viral load compared to the subject's peak sputum lavage viral load prior to treatment.
81. The method of embodiment 47, wherein treating the respiratory enterovirus in the subject comprises reducing or preventing an increase in the subject's AUC nasal viral load compared to the subject's AUC nasal viral load prior to treatment.
82. The method of embodiment 47, wherein treating the respiratory enterovirus in the subject comprises reducing or preventing an increase in the subject's AUC sputum viral load compared to the subject's AUC sputum viral load prior to treatment.
83. The method of embodiment 47, wherein treating the respiratory enterovirus in the subject comprises reducing the duration of viral shedding.
84. The method of embodiment 47, wherein treating the respiratory enterovirus in the subject comprises reducing or preventing an increase in the subject's peak sputum bacterial load compared to the subject's peak sputum lavage bacterial load prior to treatment.
85. The method of embodiment 47, wherein treating the respiratory enterovirus in the subject comprises reducing or preventing an increase in the subject's AUC sputum bacterial load compared to the subject's AUC sputum bacterial load prior to treatment.
86. The method of embodiment 47, wherein treating the respiratory enterovirus in the subject comprises reducing the number of days wherein the subject is positive for bacteria in the sputum.
87. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days,
   wherein orally administering the loading dose to the subject results in a Cₘₐₓ of about 2400 ng/mL to about 3750 ng/mL of vapendavir;
   wherein the subject is in a fed state when orally administering the loading dose and the maintenance doses; and
   wherein the subject is taking an acid reducer when orally administering the loading dose and the maintenance doses;
   wherein the respiratory enterovirus in the subject is treated.
88. The method of embodiment 87, wherein orally administering the loading dose to the subject results in a Cₘₐₓ of about 3000 ng/mL.
89. The method of embodiment 87, wherein the subject has COPD.
90. The method of embodiment 87, wherein the subject has asthma.
91. The method of embodiment 87, wherein a Tₘₐₓ is about 6 hours.
92. The method of embodiment 87, wherein the loading dose is administered within about 1 to about 7 days of the subject displaying symptoms of the respiratory enterovirus.
93. The method of embodiment 87, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.
94. The method of embodiment 87, wherein the subject has COPD, and wherein treating the respiratory condition in the subject comprises decreasing a Evaluating Respiratory Symptoms in COPD Score (E-RS) in the subject compared to a Evaluating Respiratory Symptoms in COPD Score (E-RS) in the subject prior to treatment.
95. The method of embodiment 87, wherein the subject has COPD, and wherein treating the respiratory condition in the subject comprises preventing an increase in a Evaluating Respiratory Symptoms in COPD Score (E-RS) in the subject compared to a Evaluating Respiratory Symptoms in COPD Score (E-RS) in the subject prior to treatment.
96. The method of embodiment 87, wherein the subject has COPD, and wherein treating the respiratory condition in the subject comprises decreasing a peak lower respiratory symptom score (LRSS) in the subject compared to a peak lower respiratory symptom score (LRSS) in the subject prior to treatment.
97. The method of embodiment 87, wherein the subject has COPD, and wherein treating the respiratory condition in the subject comprises preventing an increase in a peak lower respiratory symptom score (LRSS) in the subject compared to a peak lower respiratory symptom score (LRSS) in the subject prior to treatment.
98. The method of embodiment 87, wherein the subject has COPD, and wherein treating the respiratory condition in the subject comprises decreasing a peak upper respiratory symptom score (URSS) in the subject compared to a peak upper respiratory symptom score (URSS) in the subject prior to treatment.
99. The method of embodiment 87, wherein the subject has COPD, and wherein treating the respiratory condition in the subject comprises preventing an increase in a peak upper respiratory symptom score (URSS) in the subject compared to a peak upper respiratory symptom score (URSS) in the subject prior to treatment.
100. The method of embodiment 87, wherein the subject has COPD, and wherein treating the respiratory condition in the subject comprises decreasing or preventing an increase in an EXAcerbation of Chronic Pulmonary Disease Tool—Patient Reported Outcome (EXACT-PRO) in the subject compared to an EXAcerbation of Chronic Pulmonary Disease Tool—Patient Reported Outcome (EXACT-PRO) in the subject prior to treatment.
101. The method of embodiment 87, wherein the subject has COPD, and wherein treating the respiratory condition in the subject comprises decreasing or preventing an increase in an EXAcerbation of Chronic Pulmonary Disease Tool—Patient Respiratory Symptoms (EXACT-RS) in the subject compared to an EXAcerbation of Chronic Pulmonary Disease Tool—Patient Respiratory Symptoms (EXACT-RS) in the subject prior to treatment.
102. The method of embodiment 87, wherein the subject has asthma, and wherein treating the respiratory enterovirus in the subject comprises decreasing the subject's Asthma Control Questionnaire (ACQ) score compared to the subject's ACQ score prior to treatment.
103. The method of embodiment 87, wherein the subject has asthma, and wherein treating the respiratory enterovirus in the subject comprises preventing an increase in the subject's Asthma Control Questionnaire (ACQ) score compared to the subject's ACQ score prior to treatment.
104. The method of embodiment 87, wherein the subject has asthma, and wherein treating the respiratory enterovirus in the subject comprises decreasing the subject's Asthma Control Test (ACT) score compared to the subject's ACT score prior to treatment.
105. The method of embodiment 87, wherein the subject has asthma, and wherein treating the respiratory enterovirus in the subject comprises preventing an increase in the subject's Asthma Control Test (ACT) score compared to the subject's ACT score prior to treatment.
106. The method of embodiment 87, wherein the subject has asthma, and wherein treating the respiratory enterovirus in the subject comprises decreasing the subject's Pediatric Asthma Symptom Scale (PASS) score compared to the subject's PASS score prior to treatment.
107. The method of embodiment 87, wherein the subject has asthma, and wherein treating the respiratory enterovirus in the subject comprises preventing an increase in the subject's Pediatric Asthma Symptom Scale (PASS) score compared to the subject's PASS score prior to treatment.
108. The method of embodiment 87, wherein the subject has asthma, and wherein treating the respiratory enterovirus in the subject comprises decreasing the subject's Asthma Severity Scoring System (ASSESS) score compared to the subject's ASSESS score prior to treatment.
109. The method of embodiment 87, wherein the subject has asthma, and wherein treating the respiratory enterovirus in the subject comprises preventing an increase in the subject's Asthma Severity Scoring System (ASSESS) score compared to the subject's ASSESS score prior to treatment.
110. The method of embodiment 87, wherein the subject has asthma, and wherein treating the respiratory enterovirus in the subject comprises decreasing the subject's Pediatric Asthma Score (PAS) score compared to the subject's PAS score prior to treatment.
111. The method of embodiment 87, wherein the subject has asthma, and wherein treating the respiratory enterovirus in the subject comprises preventing an increase in the subject's Pediatric Asthma Score (PAS) score compared to the subject's PAS score prior to treatment.
112. The method of embodiment 87, wherein the subject has asthma, and wherein treating the respiratory enterovirus in the subject comprises decreasing the subject's Pediatric Respiratory Assessment Measure (PRAM) score compared to the subject's PRAM score prior to treatment.
113. The method of embodiment 87, wherein the subject has asthma, and wherein treating the respiratory enterovirus in the subject comprises preventing an increase in the subject's Pediatric Respiratory Assessment Measure (PRAM) score compared to the subject's PRAM score prior to treatment.
114. The method of embodiment 87, wherein the subject has asthma, and wherein treating the respiratory enterovirus in the subject comprises decreasing the subject's Pediatric Asthma Risk Score (PARS) score compared to the subject's PARS score prior to treatment.
115. The method of embodiment 87, wherein the subject has asthma, and wherein treating the respiratory enterovirus in the subject comprises preventing an increase in the subject's Pediatric Asthma Risk Score (PARS) score compared to the subject's PARS score prior to treatment.
116. The method of embodiment 87, wherein treating the respiratory enterovirus in the subject comprises preventing a decrease in the subject's lung function compared to the subject's lung function prior to treatment.
117. The method of embodiment 116, wherein the subject's lung function is measured by Forced Expiratory Volume −1 (FEV₋₁; in liters and % predicted), Forced Vital Capacity (FVC; in liters and % predicted) FEV₁/FVC ratio and peak expiratory flow (PEF) or any combination thereof.
118. The method of embodiment 87, wherein treating the respiratory enterovirus in the subject comprises reducing or preventing an increase in the subject's peak nasal lavage viral load compared to the subject's peak nasal lavage viral load prior to treatment.
119. The method of embodiment 87, wherein treating the respiratory enterovirus in the subject comprises reducing or preventing an increase in the subject's peak sputum lavage viral load compared to the subject's peak sputum lavage viral load prior to treatment.
120. The method of embodiment 87, wherein treating the respiratory enterovirus in the subject comprises reducing or preventing an increase in the subject's AUC nasal viral load compared to the subject's AUC nasal viral load prior to treatment.
121. The method of embodiment 87, wherein treating the respiratory enterovirus in the subject comprises reducing or preventing an increase in the subject's AUC sputum viral load compared to the subject's AUC sputum viral load prior to treatment.
122. The method of embodiment 87, wherein treating the respiratory enterovirus in the subject comprises reducing the duration of viral shedding.
123. The method of embodiment 87, wherein treating the respiratory enterovirus in the subject comprises reducing or preventing an increase in the subject's peak sputum bacterial load compared to the subject's peak sputum lavage bacterial load prior to treatment.
124. The method of embodiment 87, wherein treating the respiratory enterovirus in the subject comprises reducing or preventing an increase in the subject's AUC sputum bacterial load compared to the subject's AUC sputum bacterial load prior to treatment.
125. The method of embodiment 87, wherein treating the respiratory enterovirus in the subject comprises reducing the number of days wherein the subject is positive for bacteria in the sputum.
126. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days,
   wherein orally administering the loading dose to the subject results in a Cₘₐₓ of about 1600 ng/mL to about 2500 ng/mL of vapendavir;
   wherein the subject is in a fasted state when orally administering the loading dose and the maintenance doses; and
   wherein the subject is taking an acid reducer when orally administering the loading dose and the maintenance doses;
   wherein the respiratory enterovirus in the subject is treated.
127. The method of embodiment 126, wherein orally administering the loading dose to the subject results in a Cₘₐₓ of about 2000 ng/mL.
128. The method of embodiment 126, wherein the subject has COPD.
129. The method of embodiment 126, wherein the subject has asthma.
130. The method of embodiment 126, wherein a Tₘₐₓ is about 2 hours.
131. The method of embodiment 126, wherein the loading dose is administered within about 1 to about 7 days of the subject displaying symptoms of the respiratory enterovirus.
132. The method of embodiment 126, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.
133. The method of embodiment 126, wherein the subject has COPD, and wherein treating the respiratory condition in the subject comprises decreasing a Evaluating Respiratory Symptoms in COPD Score (E-RS) in the subject compared to a Evaluating Respiratory Symptoms in COPD Score (E-RS) in the subject prior to treatment.
134. The method of embodiment 126, wherein the subject has COPD, and wherein treating the respiratory condition in the subject comprises preventing an increase in a Evaluating Respiratory Symptoms in COPD Score (E-RS) in the subject compared to a Evaluating Respiratory Symptoms in COPD Score (E-RS) in the subject prior to treatment.
135. The method of embodiment 126, wherein the subject has COPD, and wherein treating the respiratory condition in the subject comprises decreasing a peak lower respiratory symptom score (LRSS) in the subject compared to a peak lower respiratory symptom score (LRSS) in the subject prior to treatment.
136. The method of embodiment 126, wherein the subject has COPD, and wherein treating the respiratory condition in the subject comprises preventing an increase in a peak lower respiratory symptom score (LRSS) in the subject compared to a peak lower respiratory symptom score (LRSS) in the subject prior to treatment.
137. The method of embodiment 126, wherein the subject has COPD, and wherein treating the respiratory condition in the subject comprises decreasing a peak upper respiratory symptom score (URSS) in the subject compared to a peak upper respiratory symptom score (URSS) in the subject prior to treatment.
138. The method of embodiment 126, wherein the subject has COPD, and wherein treating the respiratory condition in the subject comprises preventing an increase in a peak upper respiratory symptom score (URSS) in the subject compared to a peak upper respiratory symptom score (URSS) in the subject prior to treatment.
139. The method of embodiment 126, wherein the subject has COPD, and wherein treating the respiratory condition in the subject comprises decreasing or preventing an increase in an EXAcerbation of Chronic Pulmonary Disease Tool—Patient Reported Outcome (EXACT-PRO) in the subject compared to an EXAcerbation of Chronic Pulmonary Disease Tool—Patient Reported Outcome (EXACT-PRO) in the subject prior to treatment.
140. The method of embodiment 126, wherein the subject has COPD, and wherein treating the respiratory condition in the subject comprises decreasing or preventing an increase in an EXAcerbation of Chronic Pulmonary Disease Tool—Patient Respiratory Symptoms (EXACT-RS) in the subject compared to an EXAcerbation of Chronic Pulmonary Disease Tool—Patient Respiratory Symptoms (EXACT-RS) in the subject prior to treatment.
141. The method of embodiment 126, wherein the subject has asthma, and wherein treating the respiratory enterovirus in the subject comprises decreasing the subject's Asthma Control Questionnaire (ACQ) score compared to the subject's ACQ score prior to treatment.
142. The method of embodiment 126, wherein the subject has asthma, and wherein treating the respiratory enterovirus in the subject comprises preventing an increase in the subject's Asthma Control Questionnaire (ACQ) score compared to the subject's ACQ score prior to treatment.
143. The method of embodiment 126, wherein the subject has asthma, and wherein treating the respiratory enterovirus in the subject comprises decreasing the subject's Asthma Control Test (ACT) score compared to the subject's ACT score prior to treatment.
144. The method of embodiment 126, wherein the subject has asthma, and wherein treating the respiratory enterovirus in the subject comprises preventing an increase in the subject's Asthma Control Test (ACT) score compared to the subject's ACT score prior to treatment.
145. The method of embodiment 126, wherein the subject has asthma, and wherein treating the respiratory enterovirus in the subject comprises decreasing the subject's Pediatric Asthma Symptom Scale (PASS) score compared to the subject's PASS score prior to treatment.
146. The method of embodiment 126, wherein the subject has asthma, and wherein treating the respiratory enterovirus in the subject comprises preventing an increase in the subject's Pediatric Asthma Symptom Scale (PASS) score compared to the subject's PASS score prior to treatment.
147. The method of embodiment 126, wherein the subject has asthma, and wherein treating the respiratory enterovirus in the subject comprises decreasing the subject's Asthma Severity Scoring System (ASSESS) score compared to the subject's ASSESS score prior to treatment.
148. The method of embodiment 126, wherein the subject has asthma, and wherein treating the respiratory enterovirus in the subject comprises preventing an increase in the subject's Asthma Severity Scoring System (ASSESS) score compared to the subject's ASSESS score prior to treatment.
149. The method of embodiment 126, wherein the subject has asthma, and wherein treating the respiratory enterovirus in the subject comprises decreasing the subject's Pediatric Asthma Score (PAS) score compared to the subject's PAS score prior to treatment.
150. The method of embodiment 126, wherein the subject has asthma, and wherein treating the respiratory enterovirus in the subject comprises preventing an increase in the subject's Pediatric Asthma Score (PAS) score compared to the subject's PAS score prior to treatment.
151. The method of embodiment 126, wherein the subject has asthma, and wherein treating the respiratory enterovirus in the subject comprises decreasing the subject's Pediatric Respiratory Assessment Measure (PRAM) score compared to the subject's PRAM score prior to treatment.
152. The method of embodiment 126, wherein the subject has asthma, and wherein treating the respiratory enterovirus in the subject comprises preventing an increase in the subject's Pediatric Respiratory Assessment Measure (PRAM) score compared to the subject's PRAM score prior to treatment.
153. The method of embodiment 126, wherein the subject has asthma, and wherein treating the respiratory enterovirus in the subject comprises decreasing the subject's Pediatric Asthma Risk Score (PARS) score compared to the subject's PARS score prior to treatment.
154. The method of embodiment 126, wherein the subject has asthma, and wherein treating the respiratory enterovirus in the subject comprises preventing an increase in the subject's Pediatric Asthma Risk Score (PARS) score compared to the subject's PARS score prior to treatment.
155. The method of embodiment 126, wherein treating the respiratory enterovirus in the subject comprises preventing a decrease in the subject's lung function compared to the subject's lung function prior to treatment.
156. The method of embodiment 155, wherein the subject's lung function is measured by Forced Expiratory Volume −1 (FEV₋₁; in liters and % predicted), Forced Vital Capacity (FVC; in liters and % predicted) FEV₁/FVC ratio and peak expiratory flow (PEF) or any combination thereof.
157. The method of embodiment 126, wherein treating the respiratory enterovirus in the subject comprises reducing or preventing an increase in the subject's peak nasal lavage viral load compared to the subject's peak nasal lavage viral load prior to treatment.
158. The method of embodiment 126, wherein treating the respiratory enterovirus in the subject comprises reducing or preventing an increase in the subject's peak sputum lavage viral load compared to the subject's peak sputum lavage viral load prior to treatment.
159. The method of embodiment 126, wherein treating the respiratory enterovirus in the subject comprises reducing or preventing an increase in the subject's AUC nasal viral load compared to the subject's AUC nasal viral load prior to treatment.
160. The method of embodiment 126, wherein treating the respiratory enterovirus in the subject comprises reducing or preventing an increase in the subject's AUC sputum viral load compared to the subject's AUC sputum viral load prior to treatment.
161. The method of embodiment 126, wherein treating the respiratory enterovirus in the subject comprises reducing the duration of viral shedding.
162. The method of embodiment 126, wherein treating the respiratory enterovirus in the subject comprises reducing or preventing an increase in the subject's peak sputum bacterial load compared to the subject's peak sputum lavage bacterial load prior to treatment.
163. The method of embodiment 126, wherein treating the respiratory enterovirus in the subject comprises reducing or preventing an increase in the subject's AUC sputum bacterial load compared to the subject's AUC sputum bacterial load prior to treatment.
164. The method of embodiment 126, wherein treating the respiratory enterovirus in the subject comprises reducing the number of days wherein the subject is positive for bacteria in the sputum.
165. A method of treating a respiratory enterovirus in a subject in need thereof, comprising orally administering to the subject a loading dose of vapendavir sulfate in an amount sufficient to provide about 1000 mg of vapendavir free base, followed by a maintenance dose of vapendavir sulfate in an amount sufficient to provide about 1000 mg of vapendavir free base daily for about 5 days to about 7 days, wherein the subject is in a fed state when administering the loading dose and the maintenance doses, and wherein the respiratory enterovirus in the subject is treated.
166. The method of embodiment 165, wherein orally administering the loading dose to the subject results in a Cₘₐₓ of about 6400 ng/mL to about 10000 ng/mL of vapendavir.
167. The method of embodiment 165, wherein orally administering the loading dose to the subject results in a Cₘₐₓ of about 8000 ng/mL of vapendavir.
168. The method of embodiment 165, wherein the subject has COPD.
169. The method of embodiment 165, wherein the subject has asthma.
170. The method of embodiment 165, wherein the subject is not taking an acid reducer when orally administering the loading dose and the maintenance doses.
171. The method of embodiment 165, wherein the loading dose is first administered within about 1 to about 7 days of the subject displaying symptoms of the respiratory enterovirus.
172. The method of embodiment 165, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.
173. The method of embodiment 165, wherein the subject has COPD, and wherein treating the respiratory condition in the subject comprises decreasing a Evaluating Respiratory Symptoms in COPD Score (E-RS) in the subject compared to a Evaluating Respiratory Symptoms in COPD Score (E-RS) in the subject prior to treatment.
174. The method of embodiment 165, wherein the subject has COPD, and wherein treating the respiratory condition in the subject comprises preventing an increase in a Evaluating Respiratory Symptoms in COPD Score (E-RS) in the subject compared to a Evaluating Respiratory Symptoms in COPD Score (E-RS) in the subject prior to treatment.
175. The method of embodiment 165, wherein the subject has COPD, and wherein treating the respiratory condition in the subject comprises decreasing a peak lower respiratory symptom score (LRSS) in the subject compared to a peak lower respiratory symptom score (LRSS) in the subject prior to treatment.
176. The method of embodiment 165, wherein the subject has COPD, and wherein treating the respiratory condition in the subject comprises preventing an increase in a peak lower respiratory symptom score (LRSS) in the subject compared to a peak lower respiratory symptom score (LRSS) in the subject prior to treatment.
177. The method of embodiment 165, wherein the subject has COPD, and wherein treating the respiratory condition in the subject comprises decreasing a peak upper respiratory symptom score (URSS) in the subject compared to a peak upper respiratory symptom score (URSS) in the subject prior to treatment.
178. The method of embodiment 165, wherein the subject has COPD, and wherein treating the respiratory condition in the subject comprises preventing an increase in a peak upper respiratory symptom score (URSS) in the subject compared to a peak upper respiratory symptom score (URSS) in the subject prior to treatment.
179. The method of embodiment 165, wherein the subject has COPD, and wherein treating the respiratory condition in the subject comprises decreasing or preventing an increase in an EXAcerbation of Chronic Pulmonary Disease Tool—Patient Reported Outcome (EXACT-PRO) in the subject compared to an EXAcerbation of Chronic Pulmonary Disease Tool—Patient Reported Outcome (EXACT-PRO) in the subject prior to treatment.
180. The method of embodiment 165, wherein the subject has COPD, and wherein treating the respiratory condition in the subject comprises decreasing or preventing an increase in an EXAcerbation of Chronic Pulmonary Disease Tool—Patient Respiratory Symptoms (EXACT-RS) in the subject compared to an EXAcerbation of Chronic Pulmonary Disease Tool—Patient Respiratory Symptoms (EXACT-RS) in the subject prior to treatment.
181. The method of embodiment 165, wherein the subject has asthma, and wherein treating the respiratory enterovirus in the subject comprises decreasing the subject's Asthma Control Questionnaire (ACQ) score compared to the subject's ACQ score prior to treatment.
182. The method of embodiment 165, wherein the subject has asthma, and wherein treating the respiratory enterovirus in the subject comprises preventing an increase in the subject's Asthma Control Questionnaire (ACQ) score compared to the subject's ACQ score prior to treatment.
183. The method of embodiment 165, wherein the subject has asthma, and wherein treating the respiratory enterovirus in the subject comprises decreasing the subject's Asthma Control Test (ACT) score compared to the subject's ACT score prior to treatment.
184. The method of embodiment 165, wherein the subject has asthma, and wherein treating the respiratory enterovirus in the subject comprises preventing an increase in the subject's Asthma Control Test (ACT) score compared to the subject's ACT score prior to treatment.
185. The method of embodiment 165, wherein the subject has asthma, and wherein treating the respiratory enterovirus in the subject comprises decreasing the subject's Pediatric Asthma Symptom Scale (PASS) score compared to the subject's PASS score prior to treatment.
186. The method of embodiment 165, wherein the subject has asthma, and wherein treating the respiratory enterovirus in the subject comprises preventing an increase in the subject's Pediatric Asthma Symptom Scale (PASS) score compared to the subject's PASS score prior to treatment.
187. The method of embodiment 165, wherein the subject has asthma, and wherein treating the respiratory enterovirus in the subject comprises decreasing the subject's Asthma Severity Scoring System (ASSESS) score compared to the subject's ASSESS score prior to treatment.
188. The method of embodiment 165, wherein the subject has asthma, and wherein treating the respiratory enterovirus in the subject comprises preventing an increase in the subject's Asthma Severity Scoring System (ASSESS) score compared to the subject's ASSESS score prior to treatment.
189. The method of embodiment 165, wherein the subject has asthma, and wherein treating the respiratory enterovirus in the subject comprises decreasing the subject's Pediatric Asthma Score (PAS) score compared to the subject's PAS score prior to treatment.
190. The method of embodiment 165, wherein the subject has asthma, and wherein treating the respiratory enterovirus in the subject comprises preventing an increase in the subject's Pediatric Asthma Score (PAS) score compared to the subject's PAS score prior to treatment.
191. The method of embodiment 165, wherein the subject has asthma, and wherein treating the respiratory enterovirus in the subject comprises decreasing the subject's Pediatric Respiratory Assessment Measure (PRAM) score compared to the subject's PRAM score prior to treatment.
192. The method of embodiment 165, wherein the subject has asthma, and wherein treating the respiratory enterovirus in the subject comprises preventing an increase in the subject's Pediatric Respiratory Assessment Measure (PRAM) score compared to the subject's PRAM score prior to treatment.
193. The method of embodiment 165, wherein the subject has asthma, and wherein treating the respiratory enterovirus in the subject comprises decreasing the subject's Pediatric Asthma Risk Score (PARS) score compared to the subject's PARS score prior to treatment.
194. The method of embodiment 165, wherein the subject has asthma, and wherein treating the respiratory enterovirus in the subject comprises preventing an increase in the subject's Pediatric Asthma Risk Score (PARS) score compared to the subject's PARS score prior to treatment.
195. The method of embodiment 165, wherein treating the respiratory enterovirus in the subject comprises preventing a decrease in the subject's lung function compared to the subject's lung function prior to treatment.
196. The method of embodiment 195, wherein the subject's lung function is measured by Forced Expiratory Volume −1 (FEV₋₁; in liters and % predicted), Forced Vital Capacity (FVC; in liters and % predicted) FEV₁/FVC ratio and peak expiratory flow (PEF) or any combination thereof.
197. The method of embodiment 165, wherein treating the respiratory enterovirus in the subject comprises reducing or preventing an increase in the subject's peak nasal lavage viral load compared to the subject's peak nasal lavage viral load prior to treatment.
198. The method of embodiment 165, wherein treating the respiratory enterovirus in the subject comprises reducing or preventing an increase in the subject's peak sputum lavage viral load compared to the subject's peak sputum lavage viral load prior to treatment.
199. The method of embodiment 165, wherein treating the respiratory enterovirus in the subject comprises reducing or preventing an increase in the subject's AUC nasal viral load compared to the subject's AUC nasal viral load prior to treatment.
200. The method of embodiment 165, wherein treating the respiratory enterovirus in the subject comprises reducing or preventing an increase in the subject's AUC sputum viral load compared to the subject's AUC sputum viral load prior to treatment.
201. The method of embodiment 165, wherein treating the respiratory enterovirus in the subject comprises reducing the duration of viral shedding.
202. The method of embodiment 165, wherein treating the respiratory enterovirus in the subject comprises reducing or preventing an increase in the subject's peak sputum bacterial load compared to the subject's peak sputum lavage bacterial load prior to treatment.
203. The method of embodiment 165, wherein treating the respiratory enterovirus in the subject comprises reducing or preventing an increase in the subject's AUC sputum bacterial load compared to the subject's AUC sputum bacterial load prior to treatment.
204. The method of embodiment 165, wherein treating the respiratory enterovirus in the subject comprises reducing the number of days wherein the subject is positive for bacteria in the sputum.
205. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days, wherein the subject is in a fed state when administering the loading dose and the maintenance doses, and wherein the respiratory enterovirus is treated.
206. The method of embodiment 205, wherein orally administering the loading dose to the subject results in a Cₘₐₓ of about 6400 ng/mL to about 10000 ng/mL of vapendavir.
207. The method of embodiment 205, wherein orally administering the loading dose to the subject results in a Cₘₐₓ of about 8000 ng/mL of vapendavir.
208. The method of embodiment 205, wherein the subject has COPD.
209. The method of embodiment 205, wherein the subject has asthma.
210. The method of embodiment 205, wherein the subject is not taking an acid reducer when orally administering the loading dose and the maintenance doses.
211. The method of embodiment 205, wherein the loading dose is administered within about 1 to about 7 days of the subject displaying symptoms of the respiratory enterovirus.
212. The method of embodiment 205, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.
213. The method of embodiment 205, wherein the subject has COPD, and wherein treating the respiratory condition in the subject comprises decreasing a Evaluating Respiratory Symptoms in COPD Score (E-RS) in the subject compared to a Evaluating Respiratory Symptoms in COPD Score (E-RS) in the subject prior to treatment.
214. The method of embodiment 205, wherein the subject has COPD, and wherein treating the respiratory condition in the subject comprises preventing an increase in a Evaluating Respiratory Symptoms in COPD Score (E-RS) in the subject compared to a Evaluating Respiratory Symptoms in COPD Score (E-RS) in the subject prior to treatment.
215. The method of embodiment 205, wherein the subject has COPD, and wherein treating the respiratory condition in the subject comprises decreasing a peak lower respiratory symptom score (LRSS) in the subject compared to a peak lower respiratory symptom score (LRSS) in the subject prior to treatment.
216. The method of embodiment 205, wherein the subject has COPD, and wherein treating the respiratory condition in the subject comprises preventing an increase in a peak lower respiratory symptom score (LRSS) in the subject compared to a peak lower respiratory symptom score (LRSS) in the subject prior to treatment.
217. The method of embodiment 205, wherein the subject has COPD, and wherein treating the respiratory condition in the subject comprises decreasing a peak upper respiratory symptom score (URSS) in the subject compared to a peak upper respiratory symptom score (URSS) in the subject prior to treatment.
218. The method of embodiment 205, wherein the subject has COPD, and wherein treating the respiratory condition in the subject comprises preventing an increase in a peak upper respiratory symptom score (URSS) in the subject compared to a peak upper respiratory symptom score (URSS) in the subject prior to treatment.
219. The method of embodiment 205, wherein the subject has COPD, and wherein treating the respiratory condition in the subject comprises decreasing or preventing an increase in an EXAcerbation of Chronic Pulmonary Disease Tool—Patient Reported Outcome (EXACT-PRO) in the subject compared to an EXAcerbation of Chronic Pulmonary Disease Tool—Patient Reported Outcome (EXACT-PRO) in the subject prior to treatment.
220. The method of embodiment 205, wherein the subject has COPD, and wherein treating the respiratory condition in the subject comprises decreasing or preventing an increase in an EXAcerbation of Chronic Pulmonary Disease Tool—Patient Respiratory Symptoms (EXACT-RS) in the subject compared to an EXAcerbation of Chronic Pulmonary Disease Tool—Patient Respiratory Symptoms (EXACT-RS) in the subject prior to treatment.
221. The method of embodiment 205, wherein the subject has asthma, and wherein treating the respiratory enterovirus in the subject comprises decreasing the subject's Asthma Control Questionnaire (ACQ) score compared to the subject's ACQ score prior to treatment.
222. The method of embodiment 205, wherein the subject has asthma, and wherein treating the respiratory enterovirus in the subject comprises preventing an increase in the subject's Asthma Control Questionnaire (ACQ) score compared to the subject's ACQ score prior to treatment.
223. The method of embodiment 205, wherein the subject has asthma, and wherein treating the respiratory enterovirus in the subject comprises decreasing the subject's Asthma Control Test (ACT) score compared to the subject's ACT score prior to treatment.
224. The method of embodiment 205, wherein the subject has asthma, and wherein treating the respiratory enterovirus in the subject comprises preventing an increase in the subject's Asthma Control Test (ACT) score compared to the subject's ACT score prior to treatment.
225. The method of embodiment 205, wherein the subject has asthma, and wherein treating the respiratory enterovirus in the subject comprises decreasing the subject's Pediatric Asthma Symptom Scale (PASS) score compared to the subject's PASS score prior to treatment.
226. The method of embodiment 205, wherein the subject has asthma, and wherein treating the respiratory enterovirus in the subject comprises preventing an increase in the subject's Pediatric Asthma Symptom Scale (PASS) score compared to the subject's PASS score prior to treatment.
227. The method of embodiment 205, wherein the subject has asthma, and wherein treating the respiratory enterovirus in the subject comprises decreasing the subject's Asthma Severity Scoring System (ASSESS) score compared to the subject's ASSESS score prior to treatment.
228. The method of embodiment 205, wherein the subject has asthma, and wherein treating the respiratory enterovirus in the subject comprises preventing an increase in the subject's Asthma Severity Scoring System (ASSESS) score compared to the subject's ASSESS score prior to treatment.
229. The method of embodiment 205, wherein the subject has asthma, and wherein treating the respiratory enterovirus in the subject comprises decreasing the subject's Pediatric Asthma Score (PAS) score compared to the subject's PAS score prior to treatment.
230. The method of embodiment 205, wherein the subject has asthma, and wherein treating the respiratory enterovirus in the subject comprises preventing an increase in the subject's Pediatric Asthma Score (PAS) score compared to the subject's PAS score prior to treatment.
231. The method of embodiment 205, wherein the subject has asthma, and wherein treating the respiratory enterovirus in the subject comprises decreasing the subject's Pediatric Respiratory Assessment Measure (PRAM) score compared to the subject's PRAM score prior to treatment.
232. The method of embodiment 205, wherein the subject has asthma, and wherein treating the respiratory enterovirus in the subject comprises preventing an increase in the subject's Pediatric Respiratory Assessment Measure (PRAM) score compared to the subject's PRAM score prior to treatment.
233. The method of embodiment 205, wherein the subject has asthma, and wherein treating the respiratory enterovirus in the subject comprises decreasing the subject's Pediatric Asthma Risk Score (PARS) score compared to the subject's PARS score prior to treatment.
234. The method of embodiment 205, wherein the subject has asthma, and wherein treating the respiratory enterovirus in the subject comprises preventing an increase in the subject's Pediatric Asthma Risk Score (PARS) score compared to the subject's PARS score prior to treatment.
235. The method of embodiment 205, wherein treating the respiratory enterovirus in the subject comprises preventing a decrease in the subject's lung function compared to the subject's lung function prior to treatment.
236. The method of embodiment 235, wherein the subject's lung function is measured by Forced Expiratory Volume −1 (FEV₋₁; in liters and % predicted), Forced Vital Capacity (FVC; in liters and % predicted) FEV₁/FVC ratio and peak expiratory flow (PEF) or any combination thereof.
237. The method of embodiment 205, wherein treating the respiratory enterovirus in the subject comprises reducing or preventing an increase in the subject's peak nasal lavage viral load compared to the subject's peak nasal lavage viral load prior to treatment.
238. The method of embodiment 205, wherein treating the respiratory enterovirus in the subject comprises reducing or preventing an increase in the subject's peak sputum lavage viral load compared to the subject's peak sputum lavage viral load prior to treatment.
239. The method of embodiment 205, wherein treating the respiratory enterovirus in the subject comprises reducing or preventing an increase in the subject's AUC nasal viral load compared to the subject's AUC nasal viral load prior to treatment.
240. The method of embodiment 205, wherein treating the respiratory enterovirus in the subject comprises reducing or preventing an increase in the subject's AUC sputum viral load compared to the subject's AUC sputum viral load prior to treatment.
241. The method of embodiment 205, wherein treating the respiratory enterovirus in the subject comprises reducing the duration of viral shedding.
242. The method of embodiment 205, wherein treating the respiratory enterovirus in the subject comprises reducing or preventing an increase in the subject's peak sputum bacterial load compared to the subject's peak sputum lavage bacterial load prior to treatment.
243. The method of embodiment 205, wherein treating the respiratory enterovirus in the subject comprises reducing or preventing an increase in the subject's AUC sputum bacterial load compared to the subject's AUC sputum bacterial load prior to treatment.
244. The method of embodiment 205, wherein treating the respiratory enterovirus in the subject comprises reducing the number of days wherein the subject is positive for bacteria in the sputum.

### EXAMPLES

The following examples were carried out according to embodiments of the present disclosure.

### Example 1A: Preparation of Vapendavir Bisulfate

Vapendavir bisulfate was prepared at scale by suspending 4 kg of vapendavir free base in 138.4 L of tetrahydrofuran. The suspension was heated to approximately 60° C. for at least 30 minutes. Seed crystals of vapendavir bisulfate (4 g) were added. 1.1 L of 1.25 M sulfuric acid was added, and the reaction mixture was maintained at 60° C. for approximately 1 hour. The reaction mixture was then cooled for approximately 2 hours at 20° C., yielding vapendavir bisulfate as a precipitate. The precipitated vapendavir bisulfate was filtered and washed with tetrahydrofuran and then with n-heptane. After washing, the vapendavir bisulfate was collected and dried at approximately 50° C. under vacuum. The crystal habit was observed to be well-defined needles or lathe.

### Example 1B: Single Crystal Structure of Vapendavir Bisulfate

The single crystal structure of vapendavir bisulfate was obtained. The vapendavir bisulfate starting material was recrystallized by slow evaporation from isopropanol. A plate-like single crystal with high diffraction quality was selected and immersed in Paratone-N (an oil based cryoprotectant). The crystal was mounted on a mylar loop in a random orientation and immersed in a stream of liquid nitrogen at 150 K. The X-ray intensity data were measured on a Bruker D8 VENTURE (1 μS microfocus X-ray source, Cu Kα, λ=1.54178 Å, PHOTON CMOS detector) diffractometer. The frames were integrated with the Bruker SAINT software package. The integration of the data using a monoclinic unit cell yielded a total of 59613 reflections to a maximum θ angle of 67.679° (0.83 Å resolution), of which 4626 were independent (average redundancy 12.89, completeness=99.2%, Rᵢₙₜ=4.94%) and were greater than 2σ(F²). The final unit cell dimensions of a=22.223(16) Å, b=13.306(7) Å, c=7.685(4) Å, α=γ=90°, β=94.43(4)°, cell volume=2266(2) Å³, are based upon the refinement of the XYZ-centroids of 383 reflections above 20 σ(I) with 5.194°<θ<42.469°. Data were corrected for absorption effects using the Multi-Scan method (SADABS). The absorption coefficient pa of this material is 1.712 mm⁻¹ at this wavelength (1.54178 Å). The calculated minimum and maximum transmission coefficients (based on crystal size) are 0.5985 and 0.7538. The agreement factor for the averaging was 3.69% based on intensity.

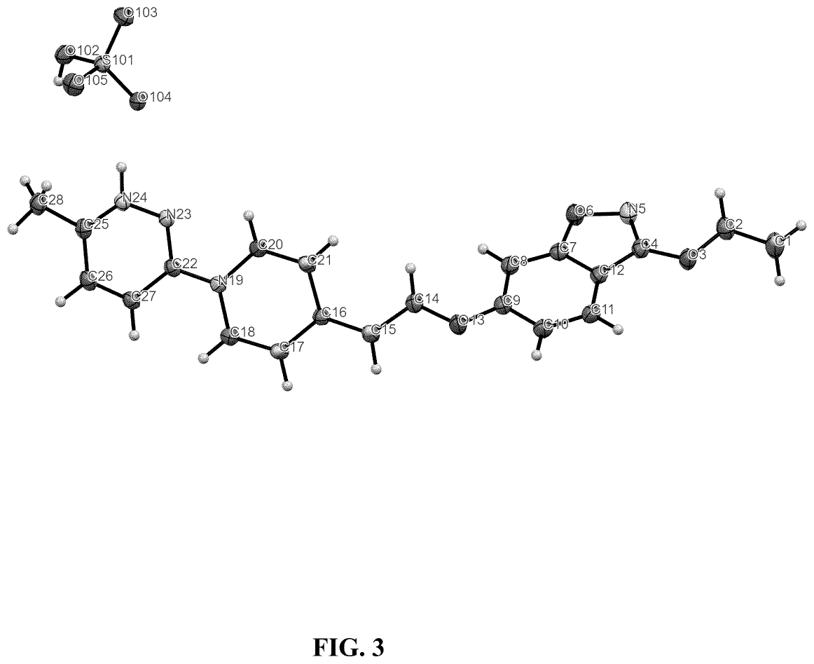

The structure was solved with the ShelXT structure solution program using Intrinsic Phasing and refined with ShelXL (Version 2014/7) refinement package using full-matrix least-squares on F² using the space group P2₁/c, with Z=4 for the formula unit, C₂₁H₂₇N₄O₃·HSO₄). All non-hydrogen atoms were refined anisotropically. The positions of the hydrogen atoms were calculated geometrically and refined using the riding model, with the exception of the hydrogen atoms bound to charged species, H24A and H991, which were located in the difference Fourier map and was allowed to refine freely to their final positions. The final anisotropic full-matrix least-squares refinement on F² with 306 variables converged at R₁=3.69%, for the observed data and wR₂=11.01% for all data. The goodness-of-fit was 1.098. The largest peak in the final difference electron density synthesis was 0.506 e⁻/Å³ and the largest hole was −0.496 e⁻/Å³. Based on the final model, the calculated density is 1.409 g/cm³ and F (000)′, 1020.56 e⁻. Two strong intermolecular interactions were observed in the structure. The first was between the protonated pyridazine nitrogen (N24) amine and bisulfate oxygen O104, associated with the ionic attraction between the bisulfate and the localized charge generated by the moiety it protonated. The donor-acceptor distance in this interaction is 2.741 Angstroms. The second interaction was hydrogen bonding between adjacent bisulfate moieties, with the protonated O102 and non-protonated O105, with a donor-acceptor distance of 2.538 Angstroms. FIG. 3 is a thermal ellipsoid rendering of the molecular structure of vapendavir bisulfate single crystal structure, displayed at 50% confidence interval, showing atomic numbering. The crystal structure indicates that the anionic species is a bisulfate (HSO₄⁻) rather than a sulfate (SO₄²⁻). For simplicity, the compound as a whole is referred to herein as a sulfate salt, and the counterion moiety is referred to as a bisulfate anion.

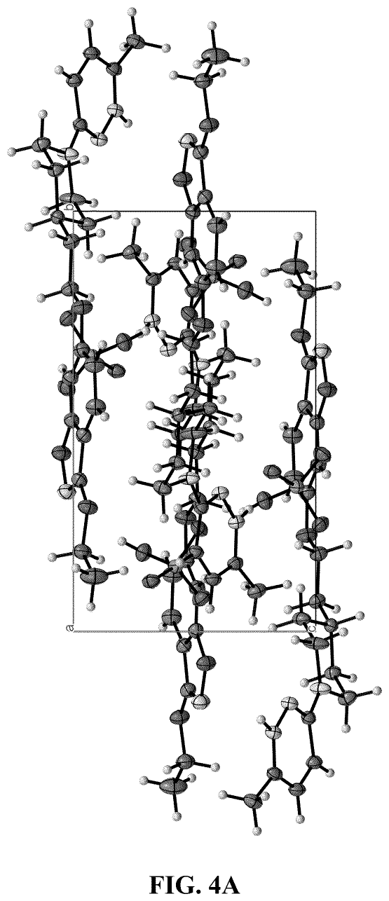

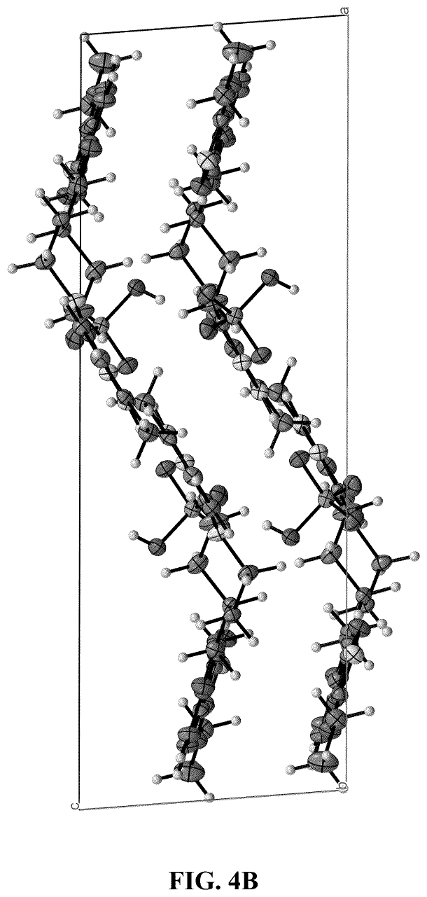

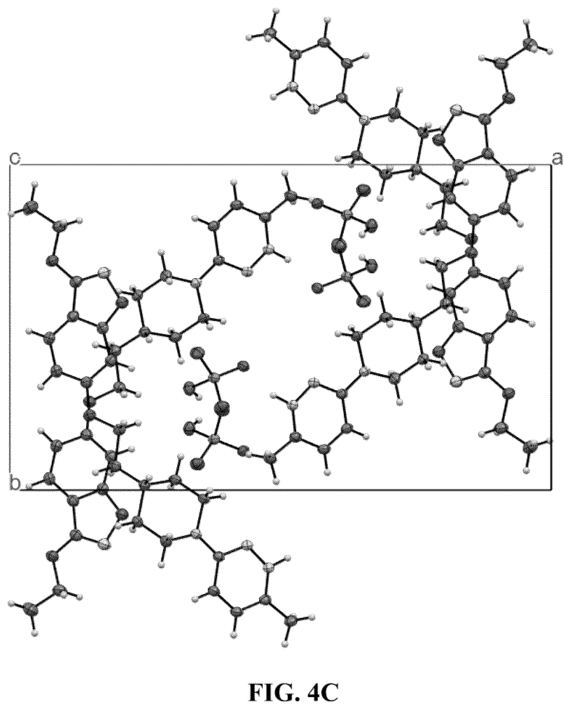

Packing diagrams of the vapendavir bisulfate crystal structure are shown in FIGS. 4A-E. FIG. 4A is the packing diagram of vapendavir bisulfate viewed along the a-axis. FIG. 4B is the packing diagram of vapendavir bisulfate viewed along the b-axis. FIG. 4C is the packing diagram of vapendavir bisulfate viewed along the c-axis. The intermolecular interactions which influence the packing arrangement are also shown. FIG. 4D shows the hydrogen-bonding network in vapendavir bisulfate, viewed down the a-axis, with hydrogen atoms omitted for clarity. FIG. 4E shows hydrogen-bonding network in vapendavir sulfate, viewed down the c-axis, with hydrogen atoms omitted for clarity. The hydrogen bonding of the bisulfate moieties in a one-dimensional chain forms a backbone with each anion coordinating with one vapendavir molecule, with symmetry giving the two equivalent orientations an appearance like ribs. These are the dominant interactions which create the packing, with the angle between the VPV sulfate molecules accommodating Van der Waals interactions between the adjacent pseudo-polymer wings, centering on the 1,2-benzisoxazole moieties. The distances and offset of these are too great to infer any π-π interactions, relegating the interactions between adjacent pseudo-polymer chains to be exclusively steric in nature. Without wishing to be bound by theory, it is contemplated that the packing of the presently disclosed crystal structure may lead to improved solubility relative to other packing motifs. Solubility, particularly is aqueous media, is an important parameter for pharmaceutical performance, and the disclosed crystalline form of vapendavir bisulfate achieves enhanced solubility relative to previously disclosed vapendavir salts.

### Example 1C: Particle Size Distribution and Dissolution of Vapendavir Bisulfate

The particle size distribution of the vapendavir bisulfate prepared according to Example 1A was evaluated by laser diffraction using a Malvern Mastersizer 3000 Particle Size Analyzer. A dispersant solution of 0.2% Span 80 (sorbitan oleate) in Isopar G (isoparaffinic hydrocarbon fluid) was prepared and used to disperse approximately 60.0 mg of vapendavir bisulfate. This dispersion was stirred and then analyzed using the Malvern Mastersizer 3000 Particle Size Analyzer, measuring first a background and then the sample in the dispersant. The % obscuration lower limit was 5.00% and upper limit was 20.00%, with a target of 13.00%±2.00%. The vapendavir bisulfate prepared by Example 1A (“as-is”) had a particle size distribution with a D90 of 34.2 μm, a D50 of 9.8 μm, and a D10 of 2.4 μm, and a specific surface area of 1.5 m²/g.

The vapendavir bisulfate of Example 1A was micronized to evaluate the effect of micronization on properties including dissolution. The micronized vapendavir bisulfate had a particle size distribution with a D90 of 2.3 μm, a D50 of 1.2 μm, and a D10 of 0.5 μm, and a specific surface area of 16.5 m²/g.

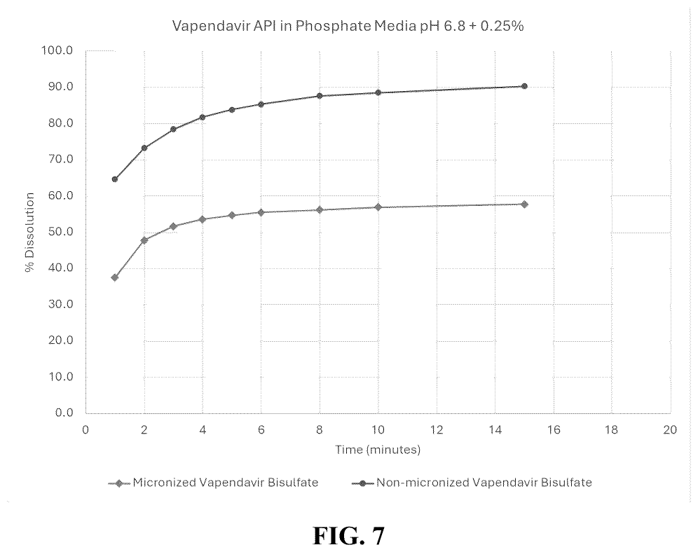

The dissolution of the “as-is” vapendavir bisulfate of Example 1A and the micronized vapendavir bisulfate were evaluated in phosphate media at pH 6.8. FIG. 7 is a graph of the solubility of vapendavir bisulfate as produced and micronized vapendavir bisulfate, according to embodiments of the present disclosure. It is commonly expected that micronized particles, having a higher surface area-to-volume ratio, will dissolve more quickly. However, as shown in FIG. 7, the “as-is” vapendavir bisulfate surprisingly dissolved more readily than the micronized vapendavir bisulfate. The “as-is” vapendavir bisulfate had a percent dissolution of approximately 90% after 15 minutes, while the micronized vapendavir bisulfate had a percent dissolution of approximately 55% after 15 minutes. This unexpected result may suggest the suitability of vapendavir bisulfate prepared by the methods disclosed herein for incorporation into pharmaceutical compositions.

Several batches of vapendavir bisulfate were prepared from various solvents and the particle size distribution analyzed as described herein. The results of this analysis are shown in TABLE A.

TABLE A

| | | | | | Bulk | Tap | |
|---|---|---|---|---|---|---|---|
| Process/ | D10 | D50 | D90 | SAA | Density | Density | |
| Solvent | (μm) | (μm) | (μm) | (m²/g) | (g/mL) | (g/mL) | Crystal Habit |
| Standard | 7.1 | 23.2 | 98.4 | | 0.04 | 0.08 | Well defined needle, lath |
| Standard + | 5 | 12.3 | 35.3 | | 0.27 | 0.33 | Small plate like primaries, |
| wet mill | | | | | | | agglomerates on the small |
| | | | | | | | side |
| THF | 5.5 | 12.8 | 27.4 | | 0.21 | 0.25 | Small plate-like primaries, |
| | | | | | | | agglomerates on the larger |
| | | | | | | | side. May disperse in |
| | | | | | | | measurement |
| MeOH | 13.1 | 54.3 | 148 | 1.644 | 0.24 | 0.28 | Larger plate like primaries, |
| | | | | | | | larger agglomerates. Not |
| | | | | | | | dispersing in measurement |
| EtOH | 11.3 | 39.8 | 101 | | 0.19 | 0.26 | Larger plate like |
| | | | | | | | primaries, larger agglomerates |
| | | | | | | | on. Not dispersing in |
| | | | | | | | measurement? |
| THF/IPA | 5.7 | 13.7 | 30.3 | 6.863 | 0.3 | 0.35 | Small plate-like primaries, |
| | | | | | | | agglomerates on the medium |
| | | | | | | | side. Probably some dispersing |
| | | | | | | | during measurement. |
| lAcetic | 2.8 | 6.2 | 14.2 | 4.401 | 0.2 | 0.29 | Very small needles, not |
| Acid/IPAc | | | | | | | highly agglomerated |

### Example 2: Absorption of Vapendavir Bisulfate

A dog PK study was performed to analyze the absorption of vapendavir bisulfate with administration of a single 200 mg/kg oral dose in a fasted state. It is contemplated that subjects with COPD may take acid reducers and requiring that subjects stop taking these medications may be undesirable. Accordingly, the study investigated whether absorption of vapendavir bisulfate is maintained with high or low pH, using famotidine (which raises pH and simulates a subject taking an acid reducer) and pentagastrin (which lowers pH and simulates a subject not taking an acid reducer), respectively.

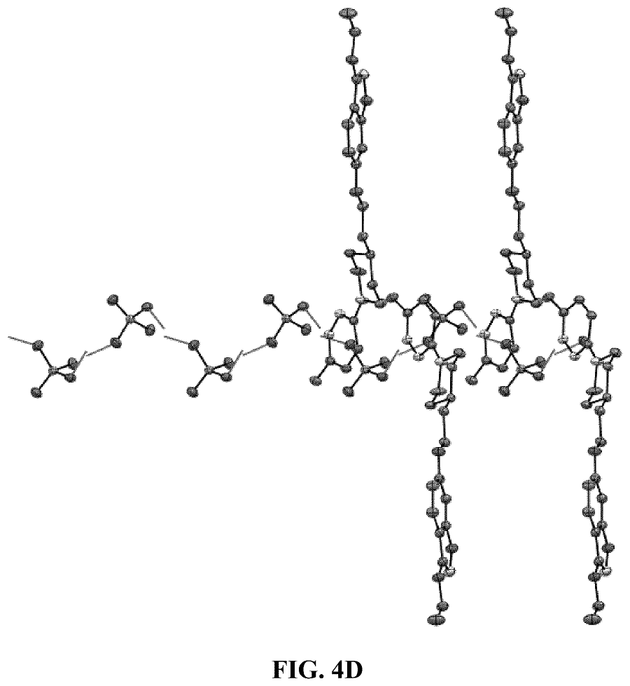

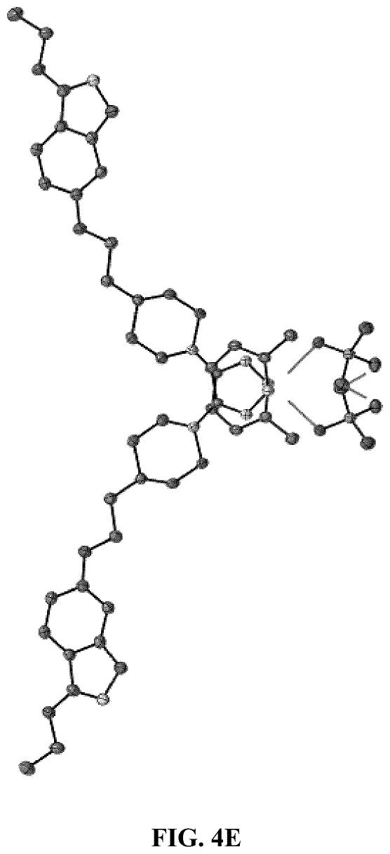

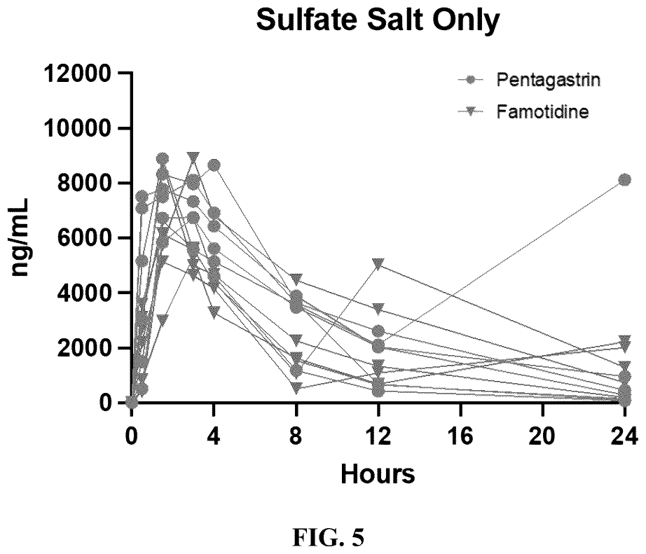

FIG. 5 is a graph of absorption of vapendavir bisulfate when co-administered with famotidine or pentagastrin. As shown in FIG. 5, the maximum absorption (in ng/mL), as well as area under the curve (AUC) and half-life, of vapendavir bisulfate was approximately the same when co-administered with famotidine and pentagastrin. This study indicates that vapendavir bisulfate can be effectively administered to subjects taking an acid reducers without negatively affecting the absorption.

### Example 3: Pharmacokinetics of Vapendavir Bisulfate in Humans

Vapendavir bisulfate was orally administered to human subjects at a loading dose of 1000 mg and concentration was evaluated over 24 hours. The resulting concentrations were evaluated in comparison to the concentrations after orally administering 1000 mg of vapendavir free base.

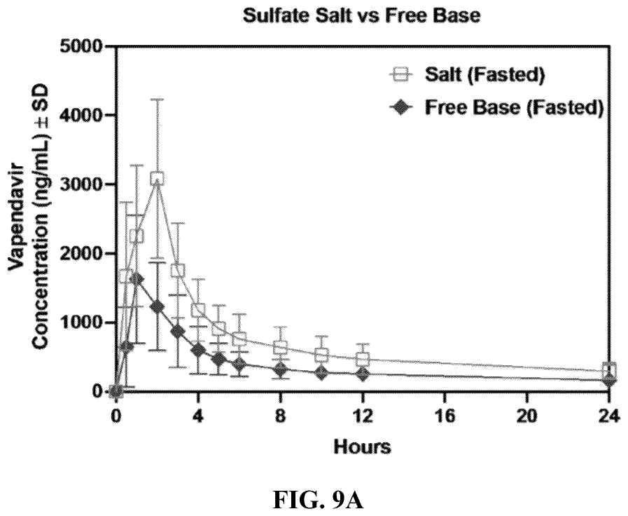

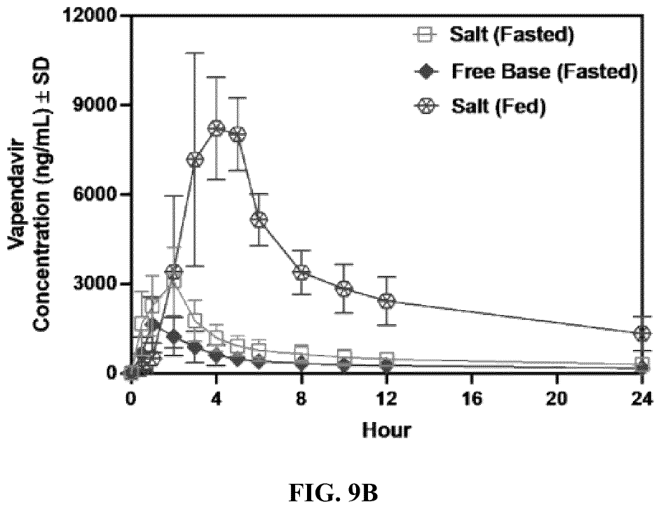

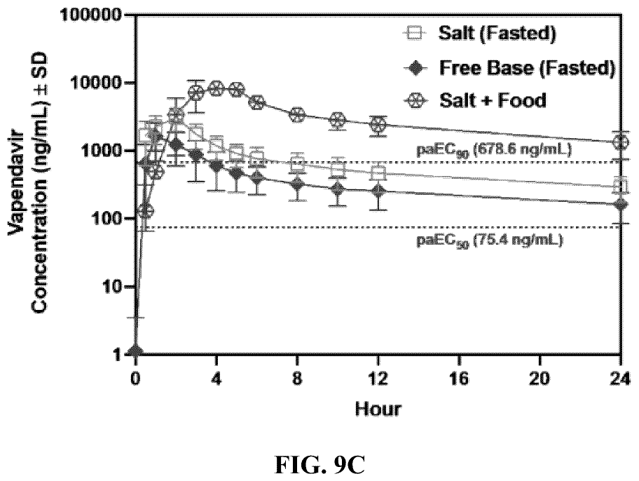

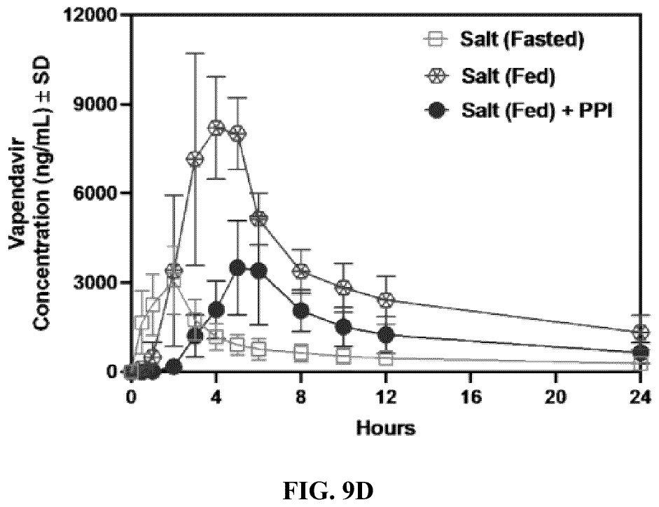

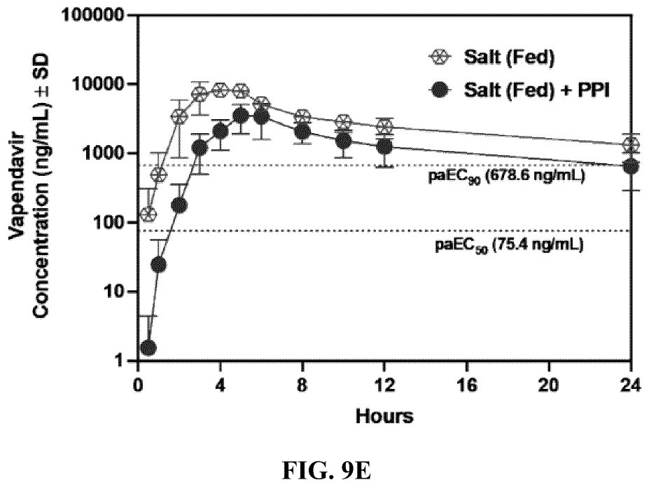

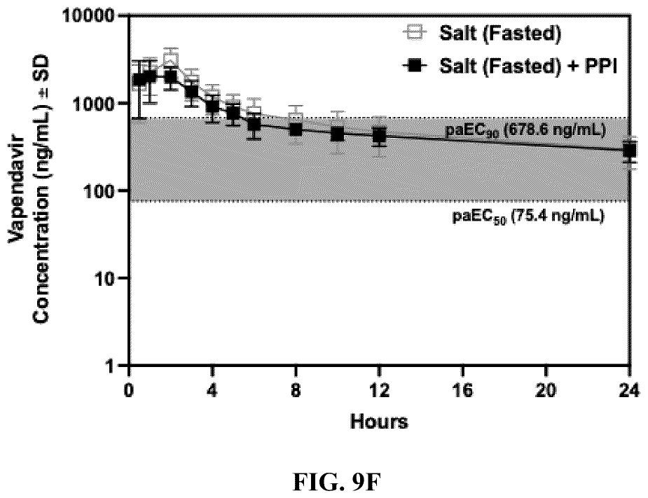

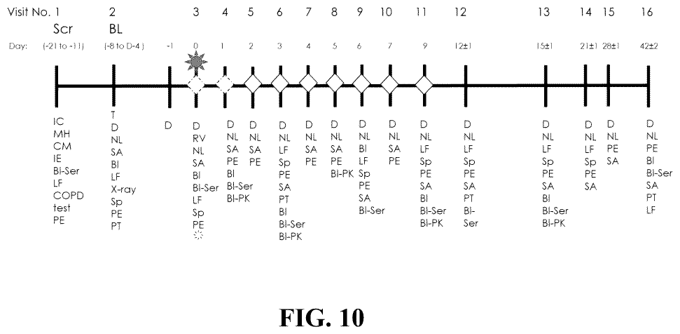

FIG. 9A shows the concentration of vapendavir bisulfate administered to a fasted subject, according to an embodiment of the present disclosure, compared to the concentration of vapendavir free base administered to a fasted subject. FIG. 9B shows the concentration of vapendavir bisulfate administered to a fasted subject and a fed subject, according to embodiments of the present disclosure, compared to the concentration of vapendavir free base administered to a fasted subject. FIG. 9C shows the concentration of vapendavir bisulfate administered to a fasted subject and a fed subject, according to embodiments of the present disclosure, compared to the concentration of vapendavir free base administered to a fasted subject, plotted on a logarithmic scale. FIG. 9D shows the concentration of vapendavir bisulfate administered to a fasted subject, a fed subject, and a fed subject taking a PPI, according to embodiments of the present disclosure. FIG. 9E shows the concentration of vapendavir bisulfate administered to a fed subject and a fed subject taking a PPI, according to embodiments of the present disclosure, plotted on a logarithmic scale. FIG. 9F shows the concentration of vapendavir bisulfate administered to a fasted subject and a fasted subject taking a PPI, according to embodiments of the present disclosure, plotted on a logarithmic scale.

As shown in FIG. 9A, orally administering vapendavir bisulfate to a fasted subject resulted in a higher concentration (Cₘₐₓ of approximately 3000 ng/mL) than orally administering vapendavir free base to a fasted subject (Cₘₐₓ of approximately 1800 ng/mL). The Tₘₐₓ for vapendavir bisulfate was approximately 2 hours, while the Tₘₐₓ for vapendavir free base was approximately 1 hour.

FIG. 9B shows that orally administering vapendavir bisulfate to a fed subject resulted in a higher Cₘₐₓ (approximately 8000 ng/mL) than oral administration of vapendavir bisulfate to a fasted subject and oral administration of vapendavir free base to a fasted subject. As further shown in FIG. 9C, oral administration of vapendavir bisulfate to both fed and fasted subjects resulted in a Cₘₐₓ above the paEC90 value of 678.6 ng/mL.

FIG. 9D compares the oral administration of vapendavir bisulfate to a fasted subject, a fed subject, and a fed subject taking a proton pump inhibitor (PPI). With vapendavir free base, it is understood that taking a PPI can result in significantly reduced drug concentrations, such that subjects may be advised to avoid PPIs when taking vapendavir free base. While taking a PPI with vapendavir bisulfate does result in reduced drug concentration (Cₘₐₓ of approximately 3000 ng/mL), the concentration is similar to the concentration in a fasted subject. As further shown in FIG. 9E, orally administering vapendavir bisulfate to a fed subject with or without a PPI resulted in drug levels above the paEC90 value. FIG. 9F shows that in fasted subjects, PPIs have very little effect on vapendavir levels. Administering vapendavir bisulfate to fasted subjects both with and without a PPI resulted in a Cₘₐₓ above the paEC₅₀ level.

TABLE 1 summarizes the Cₘₐₓ and Tₘₐₓ for the administration of vapendavir free base and vapendavir bisulfate in accordance with Example 3.

TABLE 1

| | Cₘₐₓ | Tₘₐₓ |
|---|---|---|
| | (ng/ml) | (hr) |
| Vapendavir free base, fasted, no PPI | 1800 | 1 |
| Vapendavir bisulfate, fasted, no PPI | 3000 | 2 |
| Vapendavir bisulfate, fasted, with PPI | 2000 | 2 |
| Vapendavir bisulfate, fed, no PPI | 8000 | 4 |
| Vapendavir bisulfate, fed, with PPI | 3000 | 6 |

### Example 4: a Double-Blind, Randomized, Placebo-Controlled Trial in Participants with Chronic Obstructive Pulmonary Disease (COPD) to Evaluate the Impact of Vapendavir on the Development of Lower Respiratory Tract Symptoms Following Rhinovirus Challenge

Introduction: Rhinoviruses and COPD. RVs are small RNA viruses that are ubiquitous and thought to number >180 different serotypes. RVs can be divided into RV-A, RV-B and RV-C families based on sequence homology. While normally associated with mild, self-limiting illnesses in otherwise healthy individuals, RVs can cause significant morbidity and mortality via exacerbating established chronic respiratory diseases such as asthma, COPD, or cystic fibrosis, causing complications in immunocompromised individuals such as transplant recipients, and precipitating moderate or severe illness in elderly people. Currently, there is no approved treatment or vaccine for RV infection, and very few RV specific antiviral drugs are in late-stage clinical development.

Importantly, approximately 40 to 50% of COPD exacerbations (range 10 to 70%) are linked to RV infection. The current therapies for managing COPD and treating acute exacerbations (e.g., long acting muscarinic receptor antagonists (LAMAs), long acting 32 receptor agonists (LABAs), inhaled corticosteroids (ICS), and antibacterial drugs) are again only partially effective, have significant side effects and often fail to address the underlying mechanisms of disease or triggers of exacerbation. Exacerbations are associated with increased airway inflammation; increased mucus production, delayed or deficient antiviral host defenses and increases in pathogenic respiratory bacteria.

The complex biology of COPD exacerbations has recently been illustrated using RV challenge models in patients with COPD. Mallia et al. demonstrated that RV infection induces the symptomatic, physiologic, and inflammatory features reported in naturally occurring COPD exacerbations, and that infection is associated with impaired antiviral host factors and excessive neutrophilic inflammation. This study also highlighted the temporal relationship between symptoms, virus load and mechanisms of disease, with upper respiratory symptoms beginning shortly after viral challenge, and peak lower respiratory symptoms, neutrophil inflammation, and secondary bacterial infection following at time points >1 week from onset of initial symptoms. The use of the RV challenge model in COPD participants has therefore revealed a potential treatment window for intervention of acute COPD exacerbations associated with RVs by using potent specific antiviral treatments.

Dosing. The dose selected for this study is a 1,000 mg loading dose followed by daily dosing at 500 mg BID for 7 days. The second dose may be given on day 1 (12 hr later) if the loading dose is given in the morning. The 2nd dose on day 2 (12 h later) will be 500 mg, and Days 2 through 7 (only 1st dose given on Day 7) will be 500 mg BID (12 h apart).

Study Rationale. An effective, easily administered therapy for RV infection would have the potential to reduce the serious health effects in vulnerable populations, such as those with COPD. Treatment of RV infection could reduce the severity and frequency of acute COPD exacerbations, preserve pulmonary function, prevent secondary bacterial infections, and mitigate the need for costly medical interventions, including inhaled agents, steroids, antibacterial drugs, emergency treatment, hospitalization, and mechanical ventilation. Thus, early treatment of an RV infection could potentially improve both the quality of life and longevity of COPD patients.

### Objectives

To evaluate the effectiveness of vapendavir vs. placebo
To evaluate the safety and tolerability of vapendavir vs. placebo
To evaluate the PK/PD relationship between vapendavir exposures, viral load, and other outcomes of interest

Primary Endpoint. In participants with COPD, the primary endpoint will be evaluation of the peak total lower respiratory symptom score (LRSS) in participants administered with vapendavir relative to/versus (vs) placebo as determined by Mallia et al. from day of treatment commencement until Day 42.

Secondary Endpoints. In participants with COPD, the secondary endpoints are to:
Evaluate the impact of vapendavir vs placebo on signs and symptoms of RV infection.
Evaluate the impact of vapendavir vs placebo on RV virus load and secondary bacterial infection frequency and severity.
Evaluate the impact of vapendavir vs placebo on pulmonary function tests (PFTs).
Evaluate the safety and tolerability of vapendavir vs placebo.

Exploratory Endpoints. In participants with COPD, the exploratory endpoints are to:
Evaluate the PK/PD relationship between vapendavir exposure and virus load and other outcomes of interest.
Evaluate the impact of vapendavir vs placebo on medication changes (e.g., need for antibacterial drugs, rescue medications for pulmonary exacerbations).
Evaluate the impact of vapendavir vs placebo on utilization of health care resources (e.g., urgent care visits, hospitalization).
Evaluate the impact of vapendavir vs placebo on markers of inflammation/other biomarkers.
Evaluate the impact of vapendavir vs placebo on measures of molecular quantification of respiratory viruses and bacteria.

Overall Study Design. Randomization will be 1:1 vapendavir: placebo. Participants will undergo 7 days of twice daily treatment. Treatment will commence after symptom onset or when the participant determines they have a clinical cold. After 20±4 participants have completed 7 days of treatment and undergone a further 7 days of follow up; the DSMB and Sponsor will review the data from the first 20 participants (10±2 vapendavir: 10±2 placebo) in an interim analysis comprised of available safety and efficacy endpoints and make one of the following recommendations regarding the trial:
Continue with the study
Continue with the study, but with modifications
Terminate the study

The planned end of this clinical trial is defined as the last visit of the last participant.

### Participant Selection

### Inclusion Criteria:

Male or female age ≥40 years and ≤75 years at the time of signing the informed consent form.
Confirmed diagnosis of Global Initiative for Chronic Obstructive Pulmonary Disease (GOLD) stage II COPD as defined by % predicted Forced expiratory volume in 1 second (FEV₁)≥50% and FEV₁/Forced vital capacity (FVC)<70%.
History of acute exacerbations of COPD as defined by the participant answering “yes” to the question “do your COPD symptoms get noticeably worse when you catch a cold?”
If on maintenance therapy, be medically stable for at least 2 months prior to enrolment.
Clinically stable with no exacerbations within 2 months prior to enrolment.
Absence of other viral pathogens at baseline anterior nares sample by a respiratory diagnostic panel, for example Genmark, Roche, molecular assay or similar respiratory panel.

### Exclusion Criteria:

Participants with other causes of chronic airflow limitation:
Including but not limited to: Asthma (mixed COPD and asthma is acceptable); cystic fibrosis (CF); bronchiolitis obliterans; and fibrosis such as tuberculosis (TB), idiopathic pulmonary fibrosis (IPF), or other major respiratory diagnosis (e.g., pneumonia, aspergillosis), etc.
Non-CF bronchiectasis
Any disorder, for example, cardiovascular, gastrointestinal, hepatic, renal, neurological, musculoskeletal, infectious, endocrine, metabolic, hematological, psychiatric impairment that is not medically stable, or other major physical impairment that is not considered by the investigator medically stable/controlled.
Medications that could be impacted by CYP3A4 induction and have serious complications for the participant within the treatment period without the ability to discontinue safely with a sufficient washout period before initiating vapendavir.
History of clinically significant infection (respiratory or non-respiratory) requiring antibiotic or systemic steroids >10 mg/day within 30 days prior to planned RV challenge.
Pregnant, planning to become pregnant or nursing females during and within 30 days of treatment.
Any cold symptom within the last 6 weeks such as sore throat, sneezing, rhinorrhea, malaise, nasal obstruction, or cough.
Presence (at screening) of serum rhinovirus 16 neutralizing antibody titers at greater than or equal to one in four (≥1/4) dilution.
Active allergic rhinitis, active nasal disease such as nasal polyposis, chronic rhinosinusitis etc.
Active alcohol and/or drug misuse, at the discretion of the Investigator.
Use of any over the counter cold prophylaxis products including nasal sprays, C-vitamins, zinc or Echinacea or estrogen-based hormone replacement therapy within 1 month prior to the enrolment.
Participation in other clinical trial with medical investigational product within 30 days or 5 drug half-lives (whatever is longer) prior to enrolment.
Hypersensitivity/allergy to any of the active or placebo ingredients/components.
Patients with immunosuppression (e.g., human immunodeficiency virus (HIV), transplant recipients on anti-rejection medications, those undergoing chemo- or immuno-therapy).
Other factors that in the opinion of the investigator are considered a risk.

Study Integrity. This will be a double-blind study, placebo-controlled study.

Number of Participants. There will be up to 50 enrolled participants who will complete the study procedures (up to 25 per arm).

Lung Function. British Thoracic Society guidelines will be followed in the conduct of all lung function (spirometry) assessments. On the days of clinic visits, trained personnel utilizing calibrated equipment will conduct pulmonary function tests and measure FEV₁ (in liters and % predicted), FVC (in liters and % predicted) FEV₁/FVC ratio and PEF.

Intranasal RV-A16 Challenge. Participants will be challenged with 100 Tissue Culture Infective dose causing tissue culture infective dose causing 50% cytotoxicity (TCID50) of rhinovirus RV-A16 in a negative pressure chamber within ICRRU at the Imperial College Healthcare NHS Trust. This process will be performed as per the SOP or Site Operational Manual. RV-A16 will be diluted and using an atomizer (De Vilbiss Co.) and introduced into both nostrils by four inhalations. The participants will be encouraged to sit still without swallowing for several minutes to aid introduction of the challenge virus into the nasal passage.

Placebo. Placebo will be dispensed by the study team on Day 0 at the clinical visit at the study site. Participants will be given enough Placebo for the 7-day treatment phase, (e.g., Days 2 to 8). Treatment is twice daily, am and pm, 12 h apart±2 h. Placebo tablets will be matched to the active treatment and will be administered orally for 7 days as follows: 4 tablets for the initial dose as soon as symptoms are present or the subject answers “yes” to “do you think you have a cold” on the first day of dosing (e.g. Day 2), and 2 tablets, either on the same day (e.g., Day 2) or the next day. Treatment will therefore be for a total of 7 days (consisting of an initial 4-tablet dose followed by thirteen 2-tablet doses). Treatment may occur within the home, on non-clinical study days. All tablets to be swallowed with ample water and taken within 30 minutes of consuming a meal with solid food.

Vapendavir. Vapendavir will be dispensed by the study team on Day 0 at the clinical study visit at the study site. Participants will be given enough vapendavir for the 7-day treatment phase, (e.g., Days 2 to 8). Treatment is twice daily, am and pm, 12 h apart±2 h. vapendavir micronized free base tablets (250 mg each) will be given orally beginning with a 1,000 mg loading dose (4 tablets) as soon as symptoms are present or the subject answers “yes” to “do you think you have a cold” followed by 500 mg (2 tablets) either on the same day (e.g., day 2) or the next day. Treatment will therefore be for a total of 7 days consisting of an initial 1,000 mg dose (4-tablets) followed by thirteen 500 mg doses (2-tablets). Treatment may occur within the home, on non-clinical study days. All tablets to be swallowed with ample water and taken within 30 minutes of consuming a meal with solid food.

### Concomitant Medications, Treatments, and Procedures

Standard of care medications for the management of chronic COPD are allowed; examples are provided in TABLE 2. Oral steroids >10 mg/day are not allowed at screening/baseline.

TABLE 2

| Drug Class/Type | Examples |
|---|---|
| Short-acting bronchodilators | Albuterol (Proair HFA, Ventolin HFA) |
| | Levalbuterol (Xopenex) |
| | Ipratropium (Atrovent HFA) |
| | Albuterol/ipratropium (Combivent |
| | Respimat) |
| Long-acting bronchodilators | Aclidinium (Tudorza) |
| | Arformoterol (Brovana) |
| | Formoterol (Foradil, Perforomist) |
| | Glycopyrrolate (Seebri Neohaler, |
| | Lonhala Magnair) |
| | Indacaterol (Arcapta) |
| | Olodaterol (Striverdi Respimat) |
| | Revefenacin (Yupelri) |
| | Salmeterol (Serevent) |
| | Tiotropium (Spiriva) |
| | Umeclidinium (Incruse Ellipta) |
| Corticosteroids | Fluticasone (Flovent) |
| | Budesonide (Pulmicort) |
| | Prednisolone (<10 mg/day for chronic |
| | maintenance therapy; not for acute |
| | exacerbation) |
| | Beclometasone |
| LABA, LAMA and | Aclidinium/formoterol (Duaklir) |
| combination therapies: | |
| LABA and LAMA | Glycopyrrolate/formoterol (Bevespi |
| combination bronchodilator | Aerosphere) |
| therapies | Tiotropium/olodaterol (Stiolto Respimat) |
| | Umeclidinium/vilanterol (Anoro Ellipta) |
| | Glycopyrronium/indacaterol |
| Combinations of an ICS and a | Budesonide/formoterol (Symbicort) |
| long-acting bronchodilator | Fluticasone/salmeterol (Advair) |
| | Fluticasone/vilanterol (Breo Ellipta) |
| | Beclometasone/formoterol |
| Triple Combination Therapies | Fluticasone/umeclidinium/vilanterol |
| (ICS/LAMA/LABA) | Budesonide/glycopironium/formoterol |
| | Beclometasone/formoterol/ |
| | glycopyrronium |
| Methylxanthines | Theophylline |
| Phosphodiesterase-4 inhibitor | Roflumilast (Daliresp) |
| Mucoactive drugs | Carbocysteine |
| | Erdosteine |
| | N-acetylcysteine |
| Biologics/anti-eosinophilia | Mepolizumab (Nucala) |
| drugs | Benralizumab (Fasenra) |
| | Reslizumab (Cinqair) |
| | Dupilumab (Dupixent |
| Biologics/anti-eosinophilia | Mepolizumab (Nucala) |
| drugs | Benralizumab (Fasenra) |
| | Reslizumab (Cinqair) |
| | Dupilumab (Dupixent) |
| Other prescribed for COPD | With Sponsor/designee approval |

### Statistical Methods

Primary Endpoint. The primary statistical endpoint is the difference in change from baseline peak total LRSS as determined by Mallia et al. from day of treatment commencement until Day 42. Total LRSS each day is calculated as the mean of the total LRSS scored in the am and pm each day. The total LRSS is composed of the sum of the individual symptom scores at each scoring. Peak is defined as the highest value of the total symptom scores on any day. Change from baseline will be calculated by subtracting the mean baseline total LRSS score from every other day (Day 0 to Day 42).

Secondary Endpoints. Some, but not all secondary objectives will be adequately powered with 50 participants per arm. The secondary objectives are:
1. Evaluate the impact of vapendavir vs placebo on signs and symptoms of RV infection. All will be calculated as a change from baseline:
   Difference in peak total URSS; day of treatment commencement to Day 42
   Difference in peak EXACT-RS; day of treatment commencement to Day 42
   Difference in peak EXACT-PRO; day of treatment commencement to Day 42
   Difference in AUC LRSS; day of treatment commencement to Day 42
   Difference in AUC URSS; day of treatment commencement to Day 42
   Difference in AUC EXACT-RS; day of treatment commencement to Day 42
   Difference in AUC EXACT-PRO; day of treatment commencement to Day 42
   Number of days any URSS; day of treatment commencement to Day 42
   Number of days any LRSS; day of treatment commencement to Day 42
   Difference in peak total EXACT-RS and EXACT-PRO score day of treatment commencement to Day 14
   Difference in peak total EXACT-RS and EXACT-PRO score day of treatment commencement to Day 28
   Difference in peak total EXACT-RS score day of treatment commencement to Day 42
   Proportion of participants with change in total EXACT-RS≥2 points from pre-dose to Day 7
   Proportion of participants with change in total EXACT-RS≥2 points from pre-dose to Day 14
   Proportion of participants with change in total EXACT-RS≥2 points from pre-dose to Day 28
   Mean change in AUC ER-S from pre-dose through 7 days of treatment and an additional 7 days of follow-up
   Mean change from baseline in to peak LRSS
   Difference in peak total EXACT-PRO score from pre-dose baseline to Day 7
   Difference in peak total EXACT-PRO score from pre-dose baseline to Day 14
   Difference in peak total SGRQ from pre-dose baseline to Day 28.
2. Evaluate the impact of vapendavir vs placebo on RV viral load and secondary bacterial infection
   Peak nasal lavage viral load (day of treatment commencement to to Day 42).
   Peak sputum virus load (day of treatment commencement to to Day 42).
   AUC nasal viral load (day of treatment commencement to to Day 42).
   AUC sputum viral load (day of treatment commencement to to Day 42).
   Duration of viral shedding (no. of days virus positive, Day 1 to Day 42)
   Proportion of participants positive for sputum bacteria (NHS microbiology, day of treatment commencement to to Day 42).
   Peak sputum bacterial load (NHS microbiology, day of treatment commencement to to Day 42).
   AUC sputum bacterial load (NHS microbiology, day of treatment commencement to to Day 42).
   Number of days positive for bacteria (NHS microbiology, day of treatment commencement to to Day 42).
3. Evaluate the impact of vapendavir vs placebo on lung function tests
   Maximum % change in each lung function test (FEV₁, FVC, FEV₁/FVC % predicted FEV₁, % predicted FVC) from baseline to after 7 days of treatment. and at least 7 additional days of follow-up after completing treatment.
   Difference in AUC of each lung function test through 7 days of treatment and at least 7 additional days of follow-up after completing treatment.
4. Evaluate the safety of vapendavir vs placebo
   Difference in any blood hematology test result on any day; day of treatment commencement to Day 42
   Difference in any LFTs result on any day; day of treatment commencement to Day 42
   Difference in any blood biochemistry result on any day; day of treatment commencement to Day 42
   Difference in no. of AEs or SAEs; day of treatment commencement to Day 42
   Difference in no. of participants reporting an AE or SAE; day of treatment commencement to Day 42
   Difference in no. of concomitant medications; day of treatment commencement to Day 42
   Difference in any vital signs on any day; day of treatment commencement to Day 42
   Difference in the number of participants discontinuing due to safety or tolerability reasons

Exploratory Endpoint. The exploratory objectives are not all powered by 50 participants per arm. They will include:
1. Evaluate the relationship between vapendavir exposure and viral load and other outcomes of interest.
   Correlation of nasal lavage virus load with vapendavir PK-PD
   Correlation of sputum virus load with vapendavir PK-PD
2. Evaluate the impact of vapendavir vs placebo on medication changes in participants with COPD.
   % of participants requiring increases in the frequency and/or dose of their chronic COPD medications (e.g., LAMA, LABA) from day 1 through 28 days
   Changes in beta-agonist or anti-muscarinic agent inhaler or other respiratory medications (dose/frequency of use) from day 1 through 28 days
   % of participants requiring new treatment with systemic corticosteroids from day 1 through 28 days
   % of participants requiring new treatment with antibiotics from day 1 through 28 days
   % of participants requiring new treatment with both systemic corticosteroids and antibiotics from day 1 to 28 days
3. Evaluate the impact of vapendavir vs placebo on utilization of health care resources in participants with COPD.
   % of participants diagnosed with pneumonia from day of treatment commencement to Day 28
   % of participants requiring ED evaluation
   % of participants requiring hospitalization
4. Evaluate the impact of vapendavir vs placebo on markers of inflammation/other biomarkers in participants with COPD.
   Peak serum CRP (day of treatment commencement to Day 42)
   AUC serum CRP (day of treatment commencement to Day 42)
   Peak nasal cytokine (each cytokine, day of treatment commencement to Day 42)
   AUC nasal cytokine (each cytokine, day of treatment commencement to Day 42)
   Peak sputum cytokine (each cytokine, day of treatment commencement to Day 42)
   AUC sputum cytokine (each cytokine, day of treatment commencement to Day 42)
   Peak total sputum cells (day of treatment commencement to Day 42)
   AUC total sputum cells (day of treatment commencement to Day 42)
   Peak any specific sputum cell subset (day of treatment commencement to Day 42)
   AUC any specific sputum cell subset (day of treatment commencement to Day 42)
5. Evaluate the impact of vapendavir vs placebo on measures of molecular quantification of microbes.
Peak 16S rRNA values in sputum (day of treatment commencement to Day 42)
   AUC 16S rRNA values in sputum (day of treatment commencement to Day 42)
   Peak specific pathogenic bacteria load in sputum (qPCR, day of treatment commencement to Day 42)
   AUC specific pathogenic bacteria load in sputum (qPCR, day of treatment commencement to Day 42)
   No. of pathogens in sputum or nasal lavage (respiratory diagnostic panel day of treatment commencement to Day 42)
   No. of participants positive for any pathogens in sputum or nasal lavage (respiratory diagnostic panel day of treatment commencement to Day 42)
   Compare nasal lavage and sputum RV qPCR results with respiratory diagnostic panel RV results.

### Example 5—Interim Pharmacokinetic Results from Normal Healthy Volunteers and Participants with a Diagnosis of COPD

Group A—Normal Healthy Volunteer—(Period 1/Period 2—Single dose Fast/Fed—500 mg; Period 3—MAD BID dosing for 7 days)

Group B—Participant with a diagnosis of COPD (BID dosing for 7 days)

General Considerations: Normal Healthy Volunteers (Group A) received a 500 mg dose during Periods 1 and 2 (fast/fed part). Normal Healthy Volunteers (Group A) and participants with a diagnosis of COPD (Group B) received a loading dose of 1000 mg on the morning on Day 1 followed by a 500 mg pm dose and then received 500 mg BID up to Day 7. For calculation of Pharmacokinetic (PK) parameters, BLQ values were replaced by zero (0.00) before the first measurable concentration and by missing for any other time point. PK parameters related to the elimination phase are not reliable when RSQ-Adjusted was <0.80. Whenever it was the case, AUC_{0-inf}, Residual area, T_{1/2 el}, Cl/F, and V/F were not presented.

Group A concentration Data—Normal Healthy Volunteers (Group A—Periods 1 and 2 (Fast/Fed))

Table 3 shows descriptive statistics of vapendavir plasma concentrations vs time (Group A—Periods 1 and 2 (Fast/Fed)). Measurable concentrations are observed up to 72.0 hours post-dose for most subjects under fed conditions while measurable concentrations are observed up to 48 hours post-dose for eight subjects, and 72 hours post-dose for 4 subjects under fasting conditions. Based on visual evaluation: Fed: Mean concentrations increased from pre-dose to 8 hours post-dose and decreased up to 72 h. Fasted: Mean concentrations increased from pre-dose to 1 hour post-dose and decreased up to 72 h. Higher concentrations are observed following the administration under fed conditions when compared to the fasted administration.

TABLE 3

| | | | | Relative Nominal Time (h) | | | | | |
|---|---|---|---|---|---|---|---|---|---|
| | | | | 0.00 | 0.250 | 0.500 | 1.00 | 1.50 | 2.00 |
| Group | Day | Treatment | Statistics | Concentration (ng/mL) | | | | | |
| A | 1 | Single dose of | N | 8.00 | 8.00 | 8.00 | 8.00 | 8.00 | 8.00 |
| | | 500 mg VPV, | Mean | 0.00 | 0.709 | 25.5 | 256 | 1080 | 1700 |
| | | after a high- | SD | 0.00 | 2.00 | 43.2 | 186 | 753 | 1240 |
| | | fat meal | CV % | NC | 283 | 177 | 72.5 | 70.0 | 72.5 |
| | | | Geometric | NC | NC | NC | 195 | 873 | 1340 |
| | | | Mean | | | | | | |
| | | | Geometric | NC | NC | NC | 98.6 | 82.7 | 86.9 |
| | | | CV % | | | | | | |
| A | 1 | Single dose of | N | 9.00 | 9.00 | 9.00 | 9.00 | 9.00 | 9.00 |
| | | 500 mg VPV, | Mean | 0.00 | 13.8 | 489 | 687 | 653 | 612 |
| | | fasted | SD | 0.00 | 21.3 | 538 | 642 | 525 | 433 |
| | | | CV % | NC | 155 | 110 | 93.5 | 80.4 | 70.7 |
| | | | Geometric | NC | NC | NC | 354 | 400 | 446 |
| | | | Mean | | | | | | |
| | | | Geometric | NC | NC | NC | 309 | 206 | 122 |
| | | | CV % | | | | | | |

| | Relative Nominal Time (h) | | | | | | | |
|---|---|---|---|---|---|---|---|---|
| | 3.00 | 4.00 | 8.00 | 12.0 | 24.0 | 36.0 | 48.0 | 72.0 |
| Group | Concentration (ng/mL) | | | | | | | |
| A | 8.00 | 8.00 | 8.00 | 8.00 | 8.00 | 8.00 | 8.00 | 7.00 |
| | 2040 | 2060 | 867 | 582 | 266 | 144 | 83.0 | 30.9 |
| | 1270 | 1220 | 602 | 439 | 279 | 141 | 93.4 | 35.1 |
| | 62.2 | 59.4 | 69.4 | 75.4 | 85.8 | 97.7 | 112 | 114 |
| | 1650 | 1620 | 663 | 444 | 203 | 101 | 54.6 | 70.1 |
| | 86.8 | 101 | 102 | 97.4 | 89.4 | 106 | 116 | 123 |
| A | 9.00 | 9.00 | 9.00 | 9.00 | 9.00 | 9.00 | 8.00 | 4.00 |
| | 539 | 334 | 233 | 168 | 96.1 | 55.2 | 35.0 | 24.7 |
| | 379 | 271 | 143 | 110 | 75.2 | 64.0 | 33.8 | 13.5 |
| | 70.3 | 70.5 | 61.8 | 65.9 | 83.4 | 97.8 | 96.7 | 54.3 |
| | 423 | 309 | 191 | 136 | 64.5 | 25.0 | 22.7 | 21.8 |
| | 90.7 | 81.2 | 76.6 | 80.5 | 111 | 138 | 132 | 64.4 |

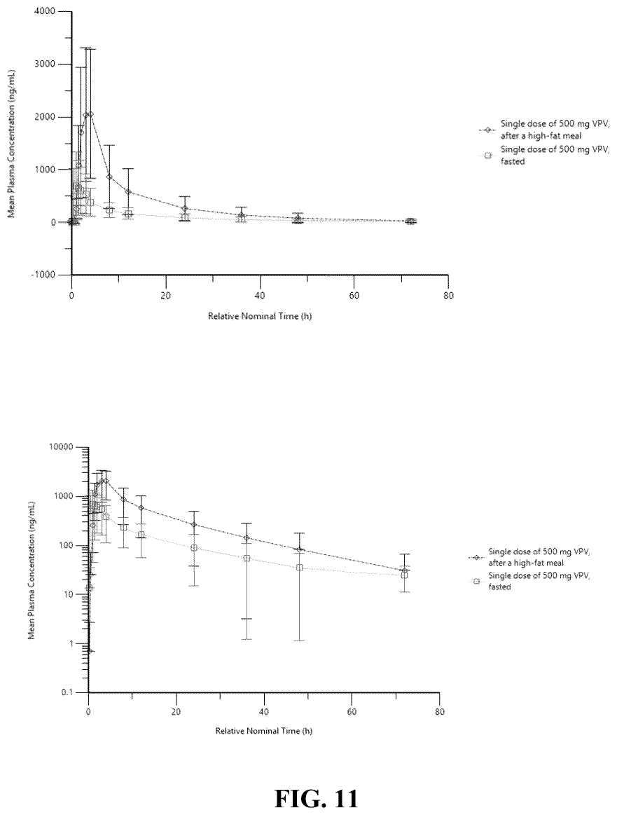

FIG. 11 depicts mean vapendavir plasma concentrations vs time profiles (Left=Linear Scale, Right=Log Scale) for Group A (Periods 1 and 2 (Fast/Fed).

Table 4 depicts descriptive statistics for PK parameters and discussion (Normal Healthy Volunteers (Group A—Periods 1 and 2 (Fast/Fed). The standard high-fat meal food intake prior to a single 500 mg VPV dose resulted in higher plasma concentrations for VPV relative to a single 500 mg dose under fasted conditions. The geometric LS mean ratio obtained are 325.36%, 329.51%, 316.11% and 274.03%. The intake of food highly increases the overall exposure of VPV. The mean peak concentration (Tₘₐₓ) was delayed under fed conditions (3.06 h) when compared to fasting conditions (1.67 h). VPV plasma elimination half-lives were similar between fed and fasted dose groups. However, CL/F and V_{z}/F are higher under fasted conditions.

TABLE 4

| | | | | AUC [symbol] | AUC [symbol] | AUC [symbol] | AUC [symbol] | C [symbol] |
|---|---|---|---|---|---|---|---|---|
| Group | Day | Treatment | Statistics | (h*ng/mL) | (h*ng/mL) | (h*ng/mL) | (h*ng/mL) | (ng/mL) |
| A | 1 | Single dose | N | 8 | 8 | 8 | 8 | 8 |
| | | of 500 mg | Mean | 23066.93 | 23694.22 | [symbol] | 18183.56 | 2460 |
| | | VPV, after | CV % | 69.30 | 70.54 | 58.55 | 62.36 | 50.47 |
| | | a high-fat | Geometric | 18538.30 | 18934.95 | 11031.35 | 14821.28 | 2130 |
| | | meal | Mean | | | | | |
| | | | Geometric | 83.04 | 83.24 | 81.09 | 82.31 | 67.67 |
| | | | CV % | | | | | |
| A | 1 | Single dose | N | 9 | 9 | 9 | 9 | 9 |
| | | of 500 mg | Mean | [symbol] | 7656.54 | 4621.14 | 5513.98 | 998 |
| | | VPV, fasted | CV % | [symbol] | 73.42 | 61.29 | 63.65 | 56.15 |
| | | | Geometric | 5614.64 | [symbol] | 3255.92 | 4461.49 | 777 |
| | | | Mean | | | | | |
| | | | Geometric | 93.71 | 92.36 | 86.43 | 83.79 | 110.99 |
| | | | CV % | | | | | |

| | | | | Residual | [symbol] | [symbol] | K_{d} | CL/F | [symbol] |
|---|---|---|---|---|---|---|---|---|---|
| Group | Day | Treatment | Statistics | Area (%) | (h) | (h) | [symbol] | (L/h) | (L) |
| A | 1 | Single dose | N | 8 | 8 | 8 | 8 | 8 | 8 |
| | | of 500 mg | Mean | 2.34 | 5.06 | 13.47 | 0.0556 | 32.85 | 655.72 |
| | | VPV, after | CV % | 62.41 | 30.78 | 24.49 | 36.09 | 70.13 | 81.47 |
| | | a high-fat | Geometric | 1.97 | 2.91 | 13.03 | 0.0532 | 26.34 | 495.07 |
| | | meal | Mean | | | | | | |
| | | | Geometric | 70.81 | 36.74 | 30.25 | 30.25 | 83.24 | 92.93 |
| | | | CV % | | | | | | |
| A | 1 | Single dose | N | 9 | 9 | 9 | 9 | 9 | 9 |
| | | of 500 mg | Mean | 4.95 | 1.67 | 15.55 | 0.0562 | 109.95 | 1942.30 |
| | | VPV, fasted | CV % | 48.65 | 61.85 | 30.01 | 38.59 | 76.05 | 78.09 |
| | | | Geometric | 4.35 | 3.40 | 12.90 | 0.0537 | 84.62 | 1574.55 |
| | | | Mean | | | | | | |
| | | | Geometric | 62.79 | 70.93 | 35.41 | 35.41 | 92.36 | 72.65 |
| | | | CV % | | | | | | |

| | | Reference | Test | Ratio¹ | | |
|---|---|---|---|---|---|---|
| PK | | Geometric | Geometric | Fed/Fast | 90% Lower | 90% Upper |
| Parameter | Units | LSM | LSM | (%) | CI (%)² | CI (%)² |
| AUC [symbol] | h*ng/mL | 5614.64 | 18267.98 | 325.36 | 173.52 | 610.07 |
| AUC₀₋₂₄ | h*ng/mL | 4461.49 | 14700.89 | 329.51 | 178.77 | 607.34 |
| AUC_{0-inf} | h*ng/mL | 5908.53 | 18677.63 | 316.11 | 169.98 | 587.88 |
| Cₘₐₓ | ng/mL | 776.94 | 2129.02 | 274.03 | 135.19 | 535.42 |

[symbol]

indicates data missing or illegible when filed

Table 5 depicts descriptive statistics of plasma concentrations vs time (Group A—Treatment C (Days 1-7). Measurable concentrations are observed up to 12.0 hours post-dose on Day 1 for all subjects. The first measurable concentration was observed at 0.500 hours for most subjects. Measurable concentrations are observed from 0.00 up to 12.0 hours post-dose on Day 4. On Day 7, measurable concentrations are observed up to 48.0 hours post-dose (end of sampling schedule for Day 7) for all subjects. Based on visual evaluation: Lower concentrations are obtained on Day 1 when compared to Day 4 and 7. Lower concentrations are obtained on Day 4 when compared to Day 7.

TABLE 5

| | | | | Relative Nominal Time (h) | | | | | | | | | |
|---|---|---|---|---|---|---|---|---|---|---|---|---|---|
| | | | | 0.00 | 0.250 | 0.500 | 1.00 | 1.50 | 2.00 | 3.00 | 4.00 | 5.00 | 12.0 |
| Group | Day | Treatment | Statistics | Concentration (ng/mL) | | | | | | | | | |
| A | 1 | VPV 1000 | N | 8 | 8 | 8 | 8 | 8 | 8 | 8 | 8 | 8 | 8 |
| | | mg am and | Mean | 0.00 | 1.59 | 21.5 | 988 | 1060 | 977 | 705 | 554 | 277 | 215 |
| | | 500 mg pm | SD | 0.00 | 4.49 | 284 | 1210 | [symbol] | 724 | 466 | 360 | 171 | 143 |
| | | | CV % | NC | 283 | 132 | 122 | 106 | 74.1 | 66.1 | 65.1 | 61.8 | 66.3 |
| | | | Geometric | NC | NC | NC | 418 | 588 | 644 | 539 | 453 | 233 | 176 |
| | | | Mean | | | | | | | | | | |
| | | | Geometric | NC | NC | NC | 359 | 239 | 177 | 115 | [symbol] | 69.4 | 78.2 |
| | | | CV % | | | | | | | | | | |
| A | 4 | VPV 500 | N | 8 | 8 | 8 | 8 | 8 | 8 | 8 | 8 | 8 | 8 |
| | | mg BID | Mean | [symbol] | 1260 | 2080 | 2059 | 1770 | 1680 | 1530 | 1400 | 1080 | 833 |
| | | | SD | 422 | 414 | 1330 | 1159 | 820 | 715 | 631 | 518 | 442 | 410 |
| | | | CV % | 33.3 | 32.9 | 63.9 | 56.0 | 46.3 | 42.5 | 41.3 | 37.0 | 40.9 | 49.3 |
| | | | Geometric | 1390 | 1190 | 1750 | 1750 | 1580 | 1540 | 1460 | 1519 | 996 | 747 |
| | | | Mean | | | | | | | | | | |
| | | | Geometric | 41.0 | 38.5 | 72.3 | 70.8 | 58.3 | 48.8 | 50.2 | 44.0 | 47.2 | 53.8 |
| | | | CV % | | | | | | | | | | |

| | | | | Relative Nominal Time (h) | | | | | | | | | | | |
|---|---|---|---|---|---|---|---|---|---|---|---|---|---|---|---|
| | | | | 0.00 | 0.250 | 0.500 | 1.00 | 1.50 | 2.00 | 3.00 | 4.00 | 8.00 | 12.0 | 24.0 | 48.0 |
| Group | Day | Treatment | Statistics | Concentration (ng/mL) | | | | | | | | | | | |
| A | 7 | VPV 500 | N | 7 | 7 | 7 | 7 | 7 | 7 | 7 | 7 | 7 | 7 | 7 | 7 |
| | | mg BID | Mean | 1770 | 1730 | 2490 | 2730 | [symbol] | 3250 | 2020 | 1880 | [symbol] | 1120 | 673 | 235 |
| | | | SD | 508 | 554 | 1370 | 1520 | 1120 | 868 | 723 | 747 | 605 | 554 | 410 | 195 |
| | | | CV % | 28.8 | 32.1 | 55.0 | 54.6 | 45.9 | 38.6 | 35.8 | 39.8 | 40.8 | 49.4 | 61.1 | 84.3 |
| | | | Geometric | 1700 | 1650 | 2180 | 2420 | 2190 | 2090 | 1910 | 1750 | 1370 | 1000 | 560 | 165 |
| | | | Mean | | | | | | | | | | | | |
| | | | Geometric | 31.5 | 34.3 | 60.2 | 63.4 | 56.1 | 43.7 | 38.2 | 41.8 | 45.9 | 55.1 | 75.7 | 118 |
| | | | CV % | | | | | | | | | | | | |

[symbol]

indicates data missing or illegible when filed

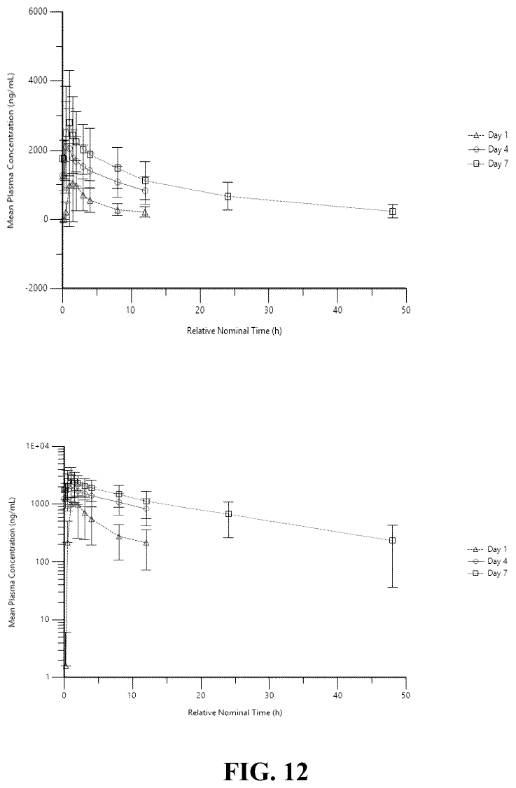

FIG. 12 shows mean vapendavir plasma concentrations vs time profiles (Left=Linear Scale, Right=Log Scale) for Group A (Treatment C) (Days 1-7). Day 1 VPV 1000 mg am and 500 mg pm; Day 4=VPV 500 mg D; Day 7=VPV 500 mg BD.

Table 6 shows descriptive statistics for PK parameters and discussion (Group A—Treatment C (Days 1 to 7)). Tmax is similar between all days. Concentrations obtained, and consequently the exposures observed, are lower following the administration of the loading dose (1000 mg) on Day 1 when compared to the data obtained following the morning dose (500 mg) on Day 4 and Day 7. For Day 7 vs Day 4, a slight accumulation was observed based RA_{AUCtau} and RA_{Cmax} with the geometric LS mean ratio obtained of 132.28 and 126.08%, respectively.

TABLE 6

| | | | | AUC [symbol] | AUC [symbol] | AUC [symbol] | AUC [symbol] | C [symbol] |
|---|---|---|---|---|---|---|---|---|
| Group | Day | Treatment | Statistics | (h*ng/mL) | (h*ng/mL) | (h*ng/mL) | (h*ng/mL) | (ng/mL) |
| A | 1 | VPV 1000 | N | 8 | 5 | 8 | 7 | 8 |
| | | mg am and | Mean | 5372.41 | 8124.70 | 5372.41 | 7508.45 | 1350 |
| | | 500 mg pm | CV % | 65.70 | 61.15 | 65.70 | 58.12 | 81.34 |
| | | | Geometric | 4270.39 | 6782.91 | 4270.89 | 6238.29 | 935 |
| | | | Mean | | | | | |
| | | | Geometric | 80.76 | 78.32 | 90.76 | [symbol] | [symbol] |
| | | | CV % | | | | | |

| | | | | Residual | [symbol] | [symbol] | K [symbol] | CL/F | [symbol] |
|---|---|---|---|---|---|---|---|---|---|
| Group | Day | Treatment | Statistics | Area (%) | (h) | (h) | [symbol] | (L/h) | (L) |
| A | 1 | VPV 1000 | N | 6 | 8 | 6 | 6 | 6 | 6 |
| | | mg am and | Mean | 23.46 | 1.94 | 5.32 | 0.1533 | 180.61 | 1396.50 |
| | | 500 mg pm | CV % | 16.10 | 48.65 | 16.98 | 16.55 | 73.70 | 79.85 |
| | | | Geometric | 23.20 | 1.77 | 5.26 | 0.1317 | 147.43 | 1119.01 |
| | | | Mean | | | | | | |
| | | | Geometric | 16.27 | 47.15 | [symbol] | 16.92 | 78.12 | 79.07 |
| | | | CV % | | | | | | |

| | | | | AUC [symbol] | AUC [symbol] | AUC [symbol] | [symbol] | [symbol] | Residual | [symbol] | [symbol] | [symbol] |
|---|---|---|---|---|---|---|---|---|---|---|---|---|
| Group | Day | Treatment | Statistics | (h*ng/mL) | (h*ng/mL) | (h*ng/mL) | (ng/mL) | (ng/mL) | Area (%) | (h) | (h) | (l/h) |
| A | 4 | VPV 500 | N | 8 | 8 | 8 | 8 | 8 | 8 | 8 | 8 | 8 |
| | | mg BID | Mean | 15366.24 | 15366.24 | 22546.40 | 2320 | 832 | 45.73 | 1.19 | 11.42 | 0.0677 |
| | | | CV % | 41.81 | 41.81 | 46.42 | 55.35 | 49.31 | 24.18 | 74.43 | 32.13 | 40.10 |
| | | | Geometric | 14095.07 | 14095.07 | 20402.07 | 2020 | 747 | 44.30 | 0.95 | 10.86 | 0.0638 |
| | | | Mean | | | | | | | | | |
| | | | Geometric | 49.18 | 49.18 | 52.54 | 65.35 | 53.77 | 29.20 | 74.92 | 36.41 | 36.41 |
| | | | CV % | | | | | | | | | |
| A | 7 | VPV 500 | N | 7 | 7 | 7 | 7 | 7 | 7 | 7 | 7 | 7 |
| | | mg BID | Mean | [symbol] | [symbol] | 31186.67 | 2930 | 1120 | 9.78 | 1.14 | 14.51 | 0.0506 |
| | | | CV % | 51.59 | 41.96 | 46.13 | 50.68 | 49.41 | [symbol] | 54.84 | 25.21 | [symbol] |
| | | | Geometric | 56547.62 | [symbol] | [symbol] | 2590 | 3000 | 8.35 | 1.00 | 14.11 | 0.0491 |
| | | | Mean | | | | | | | | | |
| | | | Geometric | 57.57 | 46.23 | 51.01 | 50.65 | 55.09 | 75.65 | 61.44 | 76.35 | 26.35 |
| | | | CV % | | | | | | | | | |

| | | | Test | Reference | Ratio³ | | |
|---|---|---|---|---|---|---|---|
| PK | | Treatment | Geometric | Geometric | Day 7/ | 90% Lower | 90% Upper |
| Parameter | Units | Comparisons | LSM | LSM | Day 4 (%) | CI (%)² | CI (%)² |
| AUC [symbol] | h*ng/mL | Day 7/Day 4 | 18645.23 | 14095.07 | 132.28 | 110.22 | 158.76 |
| C [symbol] | ng/mL | Day 7/Day 4 | 2540.79 | 2015.26 | 126.08 | 99.29 | 160.09 |

[symbol]

indicates data missing or illegible when filed

Table 7 shows descriptive statistics of plasma concentrations vs time for Group B—Treatment C (Days 1 to 7). Measurable concentrations are observed up to 12.0 hours post-dose on Day 1 for all subjects. The first measurable concentration was observed at 0.500 hours for most subjects. Measurable concentrations are observed from 0.00 up to 12.0 hours post-dose on Day 4. On Day 7, measurable concentrations are observed up to 48.0 hours post-dose (end of sampling schedule for Day 7) for all subjects. Based on visual evaluation: Lower concentrations are obtained on Day 1 when compared to Day 4 and 7. Lower concentrations are obtained on Day 4 when compared to Day 7.

TABLE 7

| | | | | Relative Nominal Time (h) | | | | | | | | | |
|---|---|---|---|---|---|---|---|---|---|---|---|---|---|
| | | | | 0.00 | 0.250 | 0.500 | 1.00 | 1.50 | 2.00 | 3.00 | 4.00 | 8.00 | 12.0 |
| Group | Day | Treatment | Statistics | Concentration (ng/mL) | | | | | | | | | |
| B-1 | 1 | VPV 1000 | N | 6 | 6 | 6 | 6 | 6 | 6 | 6 | 6 | 6 | 6 |
| | | mg am and | Mean | 0.00 | 2.35 | 499 | 1250 | 1930 | 2150 | 1740 | 1190 | 503 | 426 |
| | | 500 mg pm | SD | 0.00 | 5.76 | 495 | 772 | 1300 | 1570 | 1670 | 1080 | 603 | 438 |
| | | | CV % | NC | 245 | 99.3 | 61.6 | 67.4 | 73.1 | 95.9 | 90.6 | 120 | 103 |
| | | | Geometric | NC | NC | NC | 1020 | 1560 | 1620 | 1220 | 863 | 313 | 305 |
| | | | Mean | | | | | | | | | | |
| | | | Geometric | NC | NC | NC | 90.1 | [symbol] | 111 | 121 | 110 | 137 | 104 |
| | | | CV % | | | | | | | | | | |
| B-1 | 4 | VPV 500 | N | 6 | 6 | 6 | 6 | 6 | 6 | 6 | 6 | 6 | 6 |
| | | mg BID | Mean | 1670 | 1670 | 2430 | 2920 | 2680 | 2460 | 3270 | 1860 | 1550 | 1190 |
| | | | SD | 1260 | 1240 | 1310 | 1180 | 1220 | 1220 | 1330 | 1090 | 971 | 794 |
| | | | CV % | 75.4 | 76.2 | 53.7 | 40.5 | 45.6 | 49.8 | 56.4 | 58.8 | 62.8 | 66.6 |
| | | | Geometric | 1419 | 1370 | 2170 | 2700 | 2450 | 2250 | 2030 | 1660 | 1370 | 1040 |
| | | | Mean | | | | | | | | | | |
| | | | Geometric | 63.0 | 63.8 | 55.3 | 47.7 | 50.1 | 47.7 | 54.3 | 52.1 | 54.2 | 59.2 |
| | | | CV % | | | | | | | | | | |

| | | | | Relative Nominal Time (h) | | | | | | | | | | | |
|---|---|---|---|---|---|---|---|---|---|---|---|---|---|---|---|
| | | | | 0.00 | 0.250 | 0.500 | 1.00 | 1.50 | 2.00 | 3.00 | 4.00 | 8.00 | 12.0 | 24.0 | 48.0 |
| Group | Day | Treatment | Statistics | Concentration (ng/mL) | | | | | | | | | | | |
| B-1 | 7 | VPV 580 | N | 6 | 6 | 6 | 6 | 6 | 6 | 6 | 6 | 6 | 6 | 6 | 6 |
| | | mg BID | Mean | 1560 | 1510 | 1930 | 2800 | [symbol] | 2680 | 2160 | [symbol] | 1450 | 1040 | 760 | 285 |
| | | | SD | 665 | 637 | 499 | 1166 | 849 | 953 | 810 | 740 | 604 | 514 | 506 | 240 |
| | | | CV % | 42.5 | 45.6 | [symbol] | 41.4 | 50.0 | 35.5 | 37.5 | 40.5 | 41.6 | 49.4 | 66.7 | [symbol] |
| | | | Geometric | 1470 | 1400 | 1880 | 2630 | 2730 | 2540 | 2050 | 1730 | 1370 | 952 | 661 | 223 |
| | | | Mean | | | | | | | | | | | | |
| | | | Geometric | 38.7 | 41.4 | 24.2 | 38.4 | 29.7 | 37.8 | 33.9 | 56.0 | 37.7 | 47.3 | 57.9 | [symbol] |
| | | | CV % | | | | | | | | | | | | |

[symbol]

indicates data missing or illegible when filed

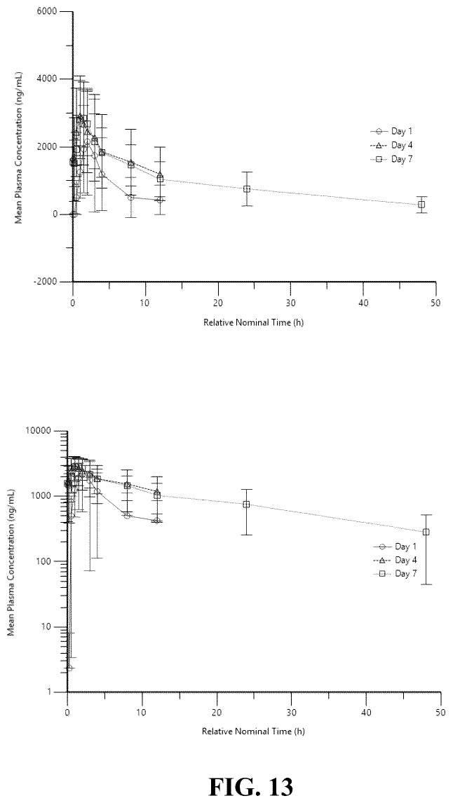

FIG. 13 depicts mean vapendavir plasma concentrations vs time profiles (Left=Linear Scale, Right=Log Scale) for Group B—Treatment C (Days 1-7). SD=Standard deviation Day 1=VPV 1000 mg am and 500 mg pm; Day 4=VPV 500 mg BID; Day 7=VPV 500 mg BID.

Table 8 shows descriptive statistics for PK parameters for Group B—Treatment C (Days 1 to 7). Tmax is similar between all days. Concentrations obtained, and consequently the exposure observed, are lower following the administration of the loading dose (1000 mg) on Day 1 when compared to the data obtained following the morning dose (500 mg) on Day 4 and Day 7. For Day 7 vs Day 4, no accumulation was observed based RA_{AUCtau} and RA_{Cmax} with the geometric LS mean ratio obtained of 100.57 and 95.38%, respectively.

TABLE 8

| | | | | AUC [symbol] | AUC [symbol] | AUC [symbol] | AUC [symbol] | C [symbol] |
|---|---|---|---|---|---|---|---|---|
| Group | Day | Treatment | Statistics | (h*ng/mL) | (h*ng/mL) | (h*ng/mL) | (h*ng/mL) | (ng/mL) |
| B-1 | 1 | VPV 1000 | N | 6 | 5 | 6 | 6 | 5 |
| | | mg am and | Mean | [symbol] | 14669.55 | 16667.31 | 13073.26 | 2340 |
| | | 500 mg am | CV % | 84.10 | 97.29 | 84.10 | 96.40 | 62.35 |
| | | | Geometric | [symbol] | 10582.42 | 8296.83 | 9954.27 | 2000 |
| | | | Mean | | | | | |
| | | | Geometric | 89.67 | 109.29 | 89.67 | 92.53 | 68.66 |
| | | | CV % | | | | | |

| | | | | Residual | T [symbol] | T [symbol] | | CL/F | |
|---|---|---|---|---|---|---|---|---|---|
| Group | Day | Treatment | Statistics | Area (%) | (h) | (h) | [symbol] | (L/h) | [symbol] |
| B-1 | 1 | VPV 1000 | N | 5 | 6 | 5 | 5 | 5 | 5 |
| | | mg am and | Mean | [symbol] | 1.75 | 5.38 | 0.1356 | 125.04 | 958.86 |
| | | 500 mg am | CV % | 24.25 | 43.33 | 25.47 | 24.53 | 80.15 | 30.75 |
| | | | Geometric | 22.26 | 1.62 | 5.24 | 0.1323 | 94.50 | 714.44 |
| | | | Mean | | | | | | |
| | | | Geometric | 24.17 | 45.48 | 25.51 | 25.51 | 109.29 | 109.35 |
| | | | CV % | | | | | | |

| | | | | AUC [symbol] | AUC [symbol] | AUC [symbol] | AUC [symbol] | C [symbol] | Residual | | | |
|---|---|---|---|---|---|---|---|---|---|---|---|---|
| Group | Day | Treauxent | Statistics | (h*ng/mL) | (h*ng/mL) | (h*ng/mL) | (h*ng/mL) | (ng/mL) | Area (%) | [symbol] | [symbol] | [symbol] |
| B-1 | 4 | TPV 500 | N | 6 | 6 | 6 | 6 | 6 | 6 | 6 | 6 | 6 |
| | | mg BID | Mean | [symbol] | 21581.16 | 32055.86 | 3280 | 1190 | 46.82 | 1.42 | 11.96 | 0.0631 |
| | | | CV % | 55.46 | 55.46 | 61.28 | 32.77 | 66.60 | 22.47 | 64.76 | 32.86 | 30.14 |
| | | | Geometric | 19521.95 | 19521.95 | 28401.17 | 3130 | 1040 | 45.81 | 1.20 | 11.45 | 0.0605 |
| | | | Mean | | | | | | | | | |
| | | | Geometric | 49.10 | 49.10 | 54.36 | 35.67 | 59.21 | 23.31 | 69.42 | 32.87 | 32.87 |
| | | | CV % | | | | | | | | | |
| B-1 | 7 | VPV 500 | N | 6 | 6 | 6 | 6 | 6 | 6 | 6 | 6 | 6 |
| | | mg BID | Mean | 42910.74 | 20613.50 | 31323.24 | 3160 | 1040 | 12.87 | 1.58 | 16.76 | 0.0427 |
| | | | CV % | 51.27 | 36.86 | 43.22 | 36.74 | 49.15 | 44.23 | 29.81 | 20.11 | 19.45 |
| | | | Geometric | [symbol] | 19634.14 | 29359.00 | 2930 | 952 | 11.75 | 1.44 | 16.49 | 0.0420 |
| | | | Mean | | | | | | | | | |
| | | | Geometric | 45.22 | 34.38 | 39.00 | 37.92 | 47.29 | 51.82 | 31.92 | 19.94 | 19.94 |
| | | | CV % | | | | | | | | | |

| | | | Reference | Test | Ratio [symbol] | | |
|---|---|---|---|---|---|---|---|
| PK | | Treatment | Geometric | Geometric | Day 7/ | 90% Lower | 90% Upper |
| Parameter | Units | Comparisons | LSM | LSM | Day 4 (%) | CI (%)² | CI (%)² |
| AUC [symbol] | h*ng/mL | Day 7/Day 4 | 19521.95 | 19634.14 | 100.57 | 83.84 | 120.65 |
| C [symbol] | ng/mL | Day 7/Day 4 | 3129.50 | 2984.94 | 95.38 | 77.61 | 117.21 |

[symbol]

indicates data missing or illegible when filed

Table 9 shows a comparison between normal healthy volunteers and participants with a diagnosis of COPD (Part A vs. Part B).

TABLE 9

| | | | | Test | Reference | Ratio¹ | | |
|---|---|---|---|---|---|---|---|---|
| | PK | | Treatment | Geometric | Geometric | HVS/ | 90% Lower | 90% Upper |
| Day | Parameter | Units | Comparisons | LSM | LSM | COPD (%) | CI (%)² | CI (%)² |
| 1 | AUC [symbol] | h*ng/mL | HVS/COPD | 4270.89 | 8296.85 | 51.48 | 20.18 | 108.65 |
| | C [symbol] | ng/mL | HVS/COPD | 935.19 | 1999.26 | 46.82 | 24.48 | 108.25 |
| 4 | AUC [symbol] | h*ng/mL | HVS/COPD | 19521.95 | 14095.07 | 72.20 | 46.14 | 112.98 |
| | C [symbol] | ng/mL | HVS/COPD | 3129.50 | 2015.26 | 64.40 | 39.52 | 104.94 |
| 7 | AUC [symbol] | h*ng/mL | HVS/COPD | 19634.14 | 19100.44 | 97.28 | 65.53 | 144.43 |
| | C [symbol] | ng/mL | HVS/COPD | 2984.94 | 2585.45 | 86.62 | 53.54 | 140.14 |

[symbol]

indicates data missing or illegible when filed

Day 1: A higher exposure was observed in participants with a diagnosis of COPD when compared to normal healthy volunteers. Indeed, the geometric LS mean ratio (normal healthy volunteers vs. participants with a diagnosis of COPD) obtained are 51.48% and 46.82%.

Day 4: A higher exposure was observed in participants with a diagnosis of COPD when compared to normal healthy volunteers. Indeed, the geometric LS mean ratio (HVS vs COPD) obtained are 72.20% and 64.40%.

For Day 7: A comparable exposure was observed in participants with a diagnosis of COPD when compared to normal healthy volunteers. Indeed, the geometric LS mean ratio and confidence interval ratios obtained are 97.28% (65.53% to 144.43%) and 86.62% (53.54% to 140.14%). However, confidence interval ratios obtained are wide demonstrating the high variability of the data obtained.

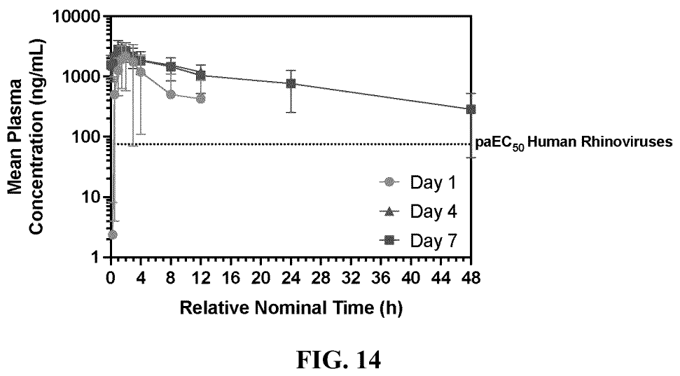

In participants with a diagnosis of COPD (Group B), administration of vapendavir for 7 days ((Day 1=VPV 1000 mg am and 500 mg pm; Day 4=VPV 500 mg BID; Day 7=VPV 500 mg BID)) results in a mean plasma concentration that exceeds the protein adjusted EC₅₀ for human rhinoviruses. See FIG. 14. This data demonstrates that the treatment regimen disclosed herein results in mean plasma concentration levels that exceed the protein adjusted EC₅₀ for human rhinoviruses suggesting it would be therapeutically effective.

Administration of 500 mg vapendavir in normal healthy volunteers that were in a fed state prior to administration results in a Cmax of 2060 ng/mL (CV % 5 ng/mL) compared to 687 ng/mL (CV % 93 ng/mL) in normal healthy volunteers that were in fasted state prior to administration.

Surprisingly, administration of a 1000 mg loading dose in COPD resulted in a Cmax of 2150 ng/mL (CV % 73 ng/mL) compared to a Cmax of 1060 ng/mL (CV % 106 ng/mL) in normal healthy volunteers suggesting that use of a loading dose of 1000 mg in participants with a diagnosis of COPD results in a higher plasma concentration and has a greater effect than in normal healthy volunteers. This is surprising, at least in part because COPD patients tend to be advanced in age (typically over the age of 70 years old) and are known to have decreased drug absorption.

This data suggests that use of a loading dose could have significant benefits for the treatment of COPD patients and in addition, treatment of patients that are in a fed state can further increase vapendavir levels compared to treatment of patients that are in a fasted state.

### Example 6—Supplementary Pharmacokinetic Results from Participants with a Diagnosis of COPD

This study was conducted to determine the food effect on vapendavir (VPV) exposure in participants with COPD including to determine the overall VPV exposure in participants with COPD, fed (loading dose followed by 7-day maintenance dose), and to determine the variation in VPV exposure in participants with COPD, fed.

Six new participants with COPD (no cross-over from previous study described in Example 5) were dosed with a 1,000 mg loading dose followed by 500 mg BID for a full 7-day course. Dosing occurred fed, with moderate fat diet, not high fat diet to better approximate actual diet of participants in their homes for the challenge study and P2/3 RCTs. PK sampling was performed on Days 2 and 3, removed Day 4; and PK sampling extended to 144 hours post-final dose on Day 7 to better characterize terminal elimination half-life.

Four participants experienced a total of eight adverse events (AEs): itching, lightheadedness, rash, increased urinary frequency (intermittent), abrasion, daytime sleepiness, bloating, and neck pain. All AEs were Grade 1 (mild) and were unrelated except for daytime sleepiness that was assessed by the investigator as possibly related.

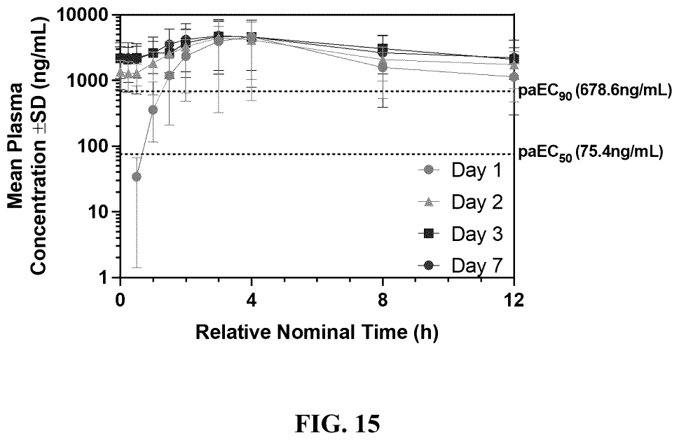

In participants with COPD dosed with a 1,000 mg loading dose followed by 500 mg BID for a full 7-day course, administration results in a mean plasma concentration that exceeds the protein adjusted EC₅₀ for human rhinoviruses. See FIG. 15 which shows VPV Concentrations over Time. This data demonstrates that the treatment regimen disclosed herein results in mean plasma concentration levels that exceed the protein adjusted EC₅₀ and EC₉₀ for human rhinoviruses suggesting it would be therapeutically effective.

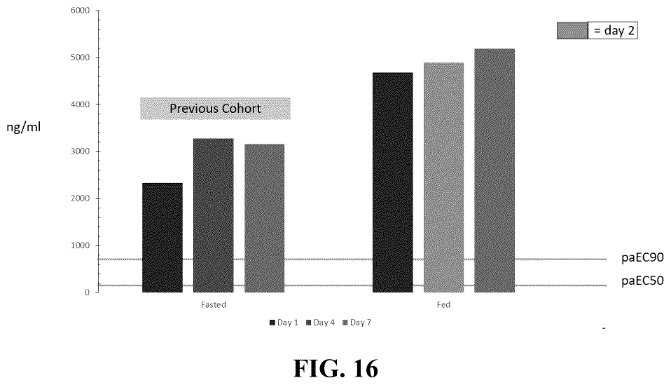

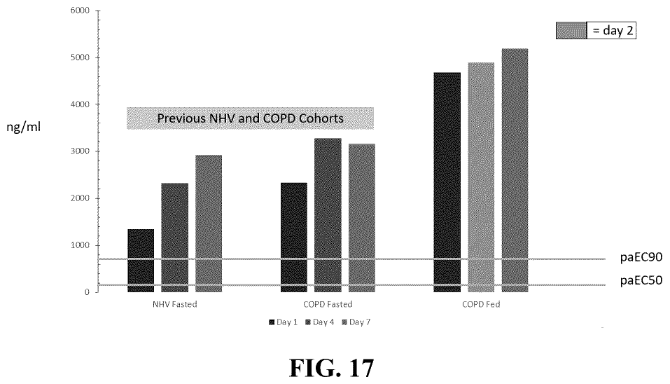

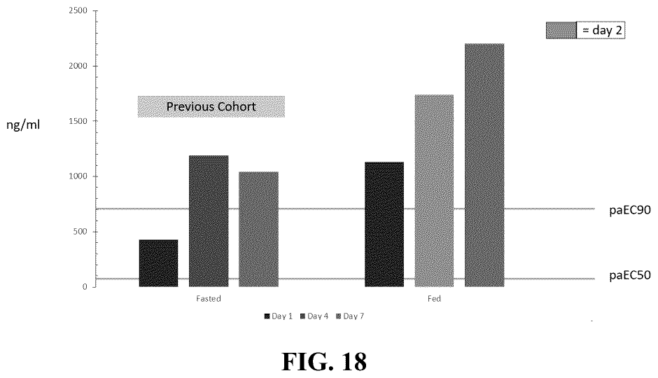

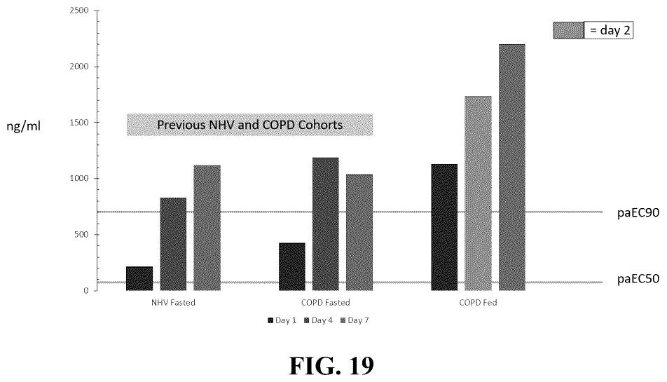

Administering VPV to participants that are fed results in increased Cmax concentrations compared with participants that were fasted. See FIG. 16 which shows the mean Cmax in fasted versus fed participants with COPD after 1, 4 and 7 days for the fasted group (Group B—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course). FIG. 17 shows the mean Cmax in fasted versus fed participants with COPD as well as normal healthy volunteers after 1, 4 and 7 days for the fasted group (Group A (Normal healthy volunteers) and Group B (COPD)—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course). FIG. 18 shows the mean Cmin (12 hours) in fasted versus fed participants with COPD after 1, 4 and 7 days for the fasted group (Group B—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course). FIG. 19 shows the mean Cmin (12 hours) in fasted versus fed participants with COPD as well as normal healthy volunteers after 1, 4 and 7 days for the fasted group (Group A (Normal healthy volunteers) and Group B (COPD)—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course).

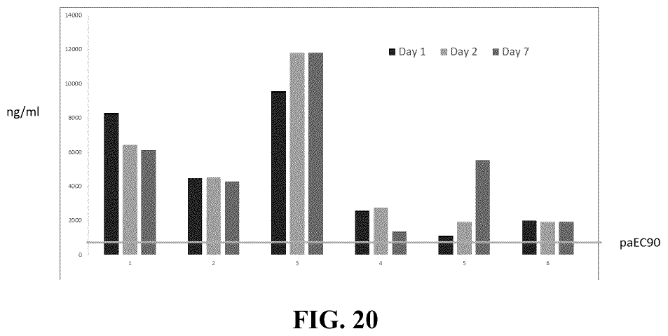

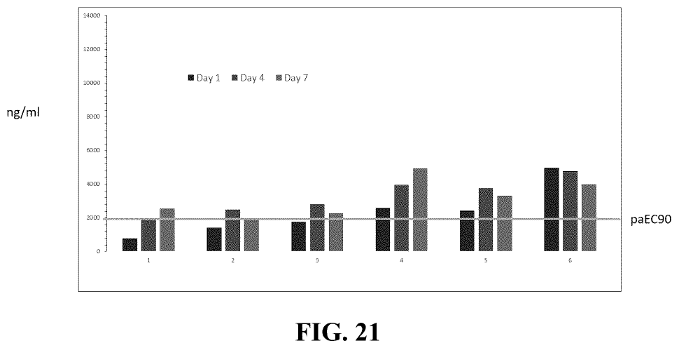

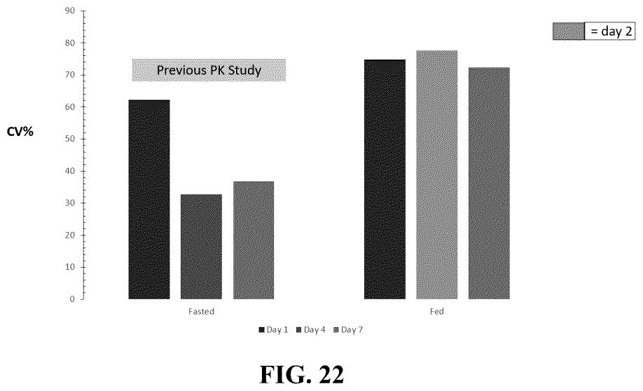

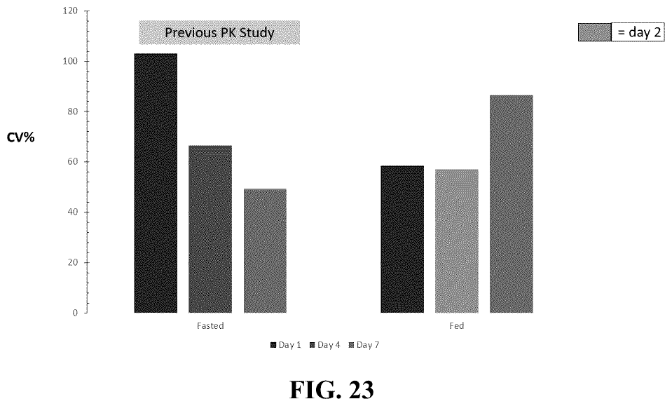

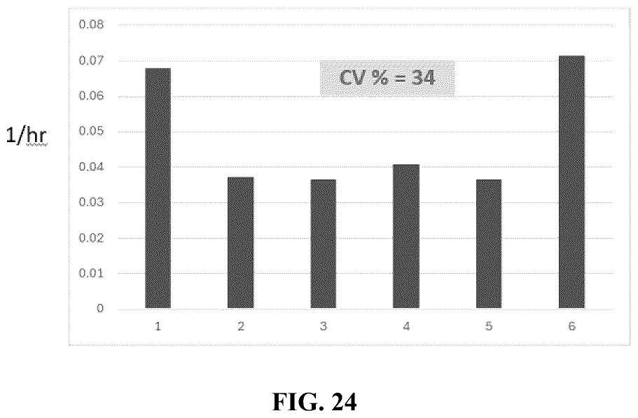

FIG. 20 shows Cmax for each day of treatment for each of the COPD subjects in the study where each subject was fed. FIG. 21 shows Cmax for each day of treatment for each of the COPD subjects in the study where each subject was fasted (Group B). FIG. 22 shows the coefficient of variation for the Cmax in fasted versus fed participants with COPD after 1, 4 and 7 days for the fasted group (Group B—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course). FIG. 23 shows the coefficient of variation for the Cmin in fasted versus fed participants with COPD after 1, 4 and 7 days for the fasted group (Group B—Treatment C (Days 1-7 (Day 1=VPV 1000 mg am and 500 mg pm; Day 4=VPV 500 mg BID; Day 7=VPV 500 mg BID)) and 1, 2, and 7 days for the fed group (1000 mg loading dose followed by 500 mg BID for a full 7-day course). FIG. 24 depicts Kₑₗ (Elimination Rate Constant) for each of the 6 Participants for the fed group (1000 mg loading dose followed by 500 mg BID for a full 7-day course) at day 7.

This data suggests that use of a loading dose could have significant benefits for the treatment of COPD patients and in addition, treatment of patients that are in a fed state can further increase vapendavir levels compared to treatment of patients that are in a fasted state.

### Example 7—Exemplary Dosing Regimens for 1000 mg Loading Dose and Thirteen Doses of 500 mg Every 12 Hours

| | |
|---|---|
| 1000 mg loading dose administered | 1000 mg loading dose administered |
| in the morning on Day 1 | in the afternoon or evening on Day 1 |
| First maintenance dose of 500 mg | First maintenance dose of 500 mg |
| administered in the evening on Day 1 | administered in the morning on Day 2 |
| Second maintenance dose of 500 mg | Second maintenance dose of 500 mg |
| administered in the morning on Day 2 | administered in the evening on Day 2 |
| Third maintenance dose of 500 mg | Third maintenance dose of 500 mg |
| administered in the evening on Day 2 | administered in the morning of Day 3 |
| Fourth maintenance dose of 500 mg | Fourth maintenance dose of 500 mg |
| administered in the morning on Day 3 | administered in the evening on Day 3 |
| Fifth maintenance dose of 500 mg | Fifth maintenance dose of 500 mg |
| administered in the evening on Day 3 | administered in the morning on Day 4 |
| Sixth maintenance dose of 500 mg | Sixth maintenance dose of 500 mg |
| administered in the morning on Day 4 | administered in the evening on Day 4 |
| Seventh maintenance dose of 500 mg | Seventh maintenance dose of 500 mg |
| administered in the evening on Day 4 | administered in the morning on Day 5 |
| Eighth maintenance dose of 500 mg | Eighth maintenance dose of 500 mg |
| administered in the morning on Day 5 | administered in the evening on Day 5 |
| Ninth maintenance dose of 500 mg | Ninth maintenance dose of 500 mg |
| administered in the evening on Day 5 | administered in the morning on Day 6 |
| Tenth maintenance dose of 500 mg | Tenth maintenance dose of 500 mg |
| administered in the morning on Day 6 | administered in the evening on Day 6 |
| Eleventh maintenance dose of 500 mg | Eleventh maintenance dose of 500 mg |
| administered in the evening on Day 6 | administered in the morning on Day 7 |
| Twelfth maintenance dose of 500 mg | Twelfth maintenance dose of 500 mg |
| administered in the morning on Day 7 | administered in the evening on Day 7 |
| Thirteenth maintenance dose of 500 mg | Thirteenth maintenance dose of 500 mg |
| administered in the evening on Day 7 | administered in the morning on Day 8 |

A DSMB review was conducted for safety of the above-described protocol. It was determined that the study continue with no changes in protocol, indicating no or minimal AE signal, and that no AEs were assessed to be a result of the study drug.

### Example 8—Challenge Study

An interim analysis of the challenge study was specified to occur after n=20+/−4 subjects completed the day 21 visit post RV-A16 challenge (of 42 total days of follow-up.) This study included data on participants having 7 days of treatment and at least 14 days of follow-up. The following population criteria were used:
Are enrolled and randomized into the study
Have been inoculated with rhinovirus serotype 16 (RV-A16) challenge virus on day 0
Have been dosed with 12/14 total VPV or placebo doses including the loading dose
Show no clinically relevant detectable rhinovirus load (less than 20 copies per microliter of cDNA) in nasal lavage or sputum at day −8 (baseline visit) and day 0
Are sampled until day 21+/−2

The efficacy population was as follows:
Randomized: n=26
   3 developed cold symptoms between baseline visit and challenge
   1 did not become symptomatic
   1 took study drug before illness
   1 had prohibited study drug discovered before challenge
Preliminary IA efficacy population: n=20
   1 had high illness scores on day 0 and PCR positive with high copy number for RV (i.e., had natural infection before challenge)
Final IA efficacy analysis population: n=19

The efficacy endpoints (to max day 21) for the interim analysis were as follows:
1. Difference peak total LRSS, day of treatment commencement to Day 21.
2. Difference in peak total URSS; day of treatment commencement to Day 21.
3. Difference in peak total EXACT-RS; day of treatment commencement to Day 21.
4. Difference in peak total EXACT-PRO; day of treatment commencement to Day 21.
5. Difference in AUC LRSS; day of treatment commencement to Day 21.
6. Difference in AUC URSS; day of treatment commencement to Day 21.
7. Difference in AUC EXACT-RS; day of treatment commencement to Day 21.
8. Difference in AUC EXACT-PRO; day of treatment commencement to Day 21.
9. Number of days any URSS; day of treatment commencement to Day 21.
10. Number of days any LRSS; day of treatment commencement to Day 21.
11. Number of days any EXACT-PRO symptoms; day of treatment commencement to day 21.
12. Number of days any EXACT-RS symptoms; day of treatment commencement to Day 21.
13. Difference in peak EXACT-RS score day of treatment commencement to Day 7
14. Difference in peak EXACT-PRO score day of treatment commencement to Day 7
15. Proportion of participants with change in total EXACT-RS≥2 points from baseline on any day from day of treatment commencement to Day 7.
16. Proportion of participants with change in total EXACT-RS≥2 points from baseline on any day from day of treatment commencement to Day 21.
17. Number of days with change in total EXACT-RS≥2 points from baseline on any day from day of treatment commencement to Day 7.
18. Number of days with change in total EXACT-RS≥2 points from baseline on any day from day of treatment commencement to Day 21.
19. Time to resolution of URSS; day of treatment commencement to Day 21.
20. Time to resolution of LRSS; day of treatment commencement to Day 21.
21. Time to resolution of EXACT-PRO symptoms; day of treatment commencement to Day 21.
22. Time to resolution of EXACT-RS symptoms; day of treatment commencement to Day 21.

Demographics are shown in TABLE 10.

TABLE 10

| | | | | |
|---|---|---|---|---|
| Male % | 4/8 | (50%) | 8/11 | (73%) |
| Age (SD) | 62.6 | (9.8) | 64.6 | (6.8) |
| Current Cigarette Use | 2/8 | (25%) | 1/11 | (9%) |
| White | 8/8 | (100%) | 9/11 | (82%) |
| FEV1 (%) (SD) | 59.8 | (11.4) | 66.1 | (14.0) |
| FEV 1/FVC (SD) | 0.52 | (0.06) | 0.58 | (0.06) |
| Baseline E-RS | 12.7 | (6.3) | 6.3 | (5.0) |

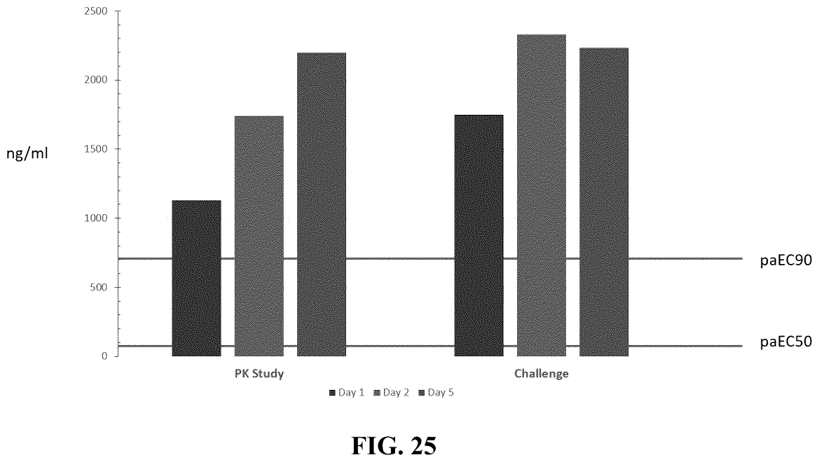

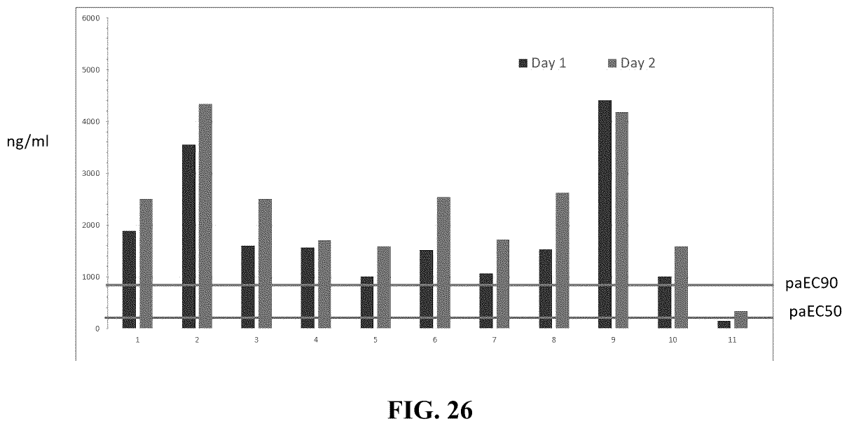

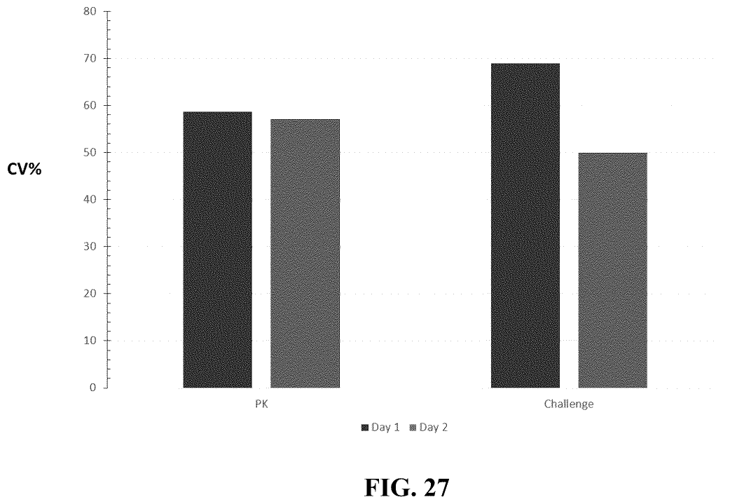

FIG. 25 shows the results of a PK study vs. challenge study in fed COPD subjects. As shown, plasma concentration in ng/mL was above paEC₅₀ and paEC₉₀ for day 1, day 2, and day 5. FIG. 26 shows challenge study results for each of the 11 VPV participants at day 1 and day 2. Plasma concentration was above paEC₅₀ and paEC₉₀ for 10 of the 11 VPV participants. FIG. 27 shows the coefficient of variation for the PK and challenge studies.

Lower respiratory tract infections were also evaluated. The primary outcome variable was peak total lower respiratory symptom score (LRSS) in participants administered with VPV relative to/versus (vs) placebo from day of treatment commencement until Day 42. This evaluation was calculated as corrected values which have the baseline value subtracted from each day's value (change from baseline).

Power calculations were performed on LRSS from two previous COPD studies using the rhinovirus challenge model and from a third, unpublished study. From these three studies power calculations showed that for a 30% difference in peak total LRSS, a minimum of 21 participants per arm are required (n=42 participants total); 25 per arm allowing for withdrawals.

LRSS (LRT) was chosen because it had been used by the investigators in all previous studies since the onset of the model in 2006. According to previous data, the peak change from baseline LRSS had the highest power to observe a statistically significant effect in a low number of patients. LRSS is not recognized by the FDA, is not used in large industry-sponsored RCTs in COPD, and has only 5 parameters. EXACT-RS (E-RS) is the preferred PRO for COPD, because it is recommended unanimously by SAB as the most relevant PRO for this indication; supported by literature, has 11 parameters versus 5 parameters in LRSS; and is more quantitative. Further, EXACT-RS is more discriminatory because it has more questions that delve into specifics around symptoms than LRSS and is qualified by FDA as an exploratory endpoint for use in randomized superiority studies to evaluate new treatments for COPD. In addition, preliminary data from the challenge model suggested that the EXACT-RS had higher power to detect a statistically significant difference than the LRSS. Finally, based on totality of FDA precedent and COVID-19 antiviral guidance, a likely pivotal endpoint will be time to sustained resolution of symptoms (based on E-RS). Evaluating Respiratory Symptoms (E-RS) is a derivative instrument of the EXACT used to assess the severity of respiratory symptoms and the effect of treatment in patients with stable COPD.

E-RS was evaluated using the following questionnaire shown in TABLE 11.

TABLE 11

| | |
|---|---|
| 1. Did your chest feel congested today? | 0. Not at all |
| | 1. Slightly |
| | 2. Moderately |
| | 3. Severely |
| | 4. Extremely |
| 2. How often did you cough today? | 0. Not at all |
| | 1. Rarely |
| | 2. Occasionally |
| | 3. Frequently |
| | 4. Almost constantly |
| 3. How much mucus (phlegm) did you bring | 0. None at all |
| up when coughing today? | 1. A little |
| | 1. Some |
| | 2. A great deal |
| | 3. A very great deal |
| | Note: |
| | score “a little” and “some” the same. |
| 4. How difficult was it to bring up mucus | 0. Not at all |
| (phlegm) today? | 1. Slightly |
| | 2. Moderately |
| | 3. Quite a bit |
| | 4. Extremely |
| 5. Did you have chest discomfort today? | 0. Not at all |
| | 1. Slight |
| | 2. Moderate |
| | 3. Severe |
| | 4. Extreme |
| 6. Did your chest feel tight today? | 0. Not at all |
| | 1. Slightly |
| | 2. Moderately |
| | 3. Severely |
| | 4. Extremely |
| 7. Were you breathless today? | 0. Not at all |
| | 1. Slightly |
| | 2. Moderately |
| | 3. Severely |
| | 4. Extremely |
| 8. Describe how breathless you were today: | 0. Unaware of breathlessness |
| | 1. Breathless during strenuous activity |
| | 2. Breathless during light activity |
| | 3. Breathless when washing or dressing |
| | 3. Present when resting |
| | Note: |
| | score “Breathless when washing or |
| | dressing” and “Present when resting” the |
| | same. |
| 9. Were you short of breath today when | 0. Not at all |
| performing your usual personal case activities | 1. Slightly |
| like washing or dressing? | 2. Moderately |
| | 3. Severely |
| | 3. Extremely |
| | 4. Too breathless to do these |
| | Note: |
| | score “Severely” and “Extremely” the |
| | same. |
| 10. Were you short of breath today when | 0. Not at all |
| performing your usual indoor activities like | 1. Slightly |
| cleaning or household work? | 2. Moderately |
| | 3. Severely |
| | 3. Extremely |
| | 3. Too breathless to do these |
| | Note: |
| | score “Severely,” “Extremely,” and |
| | “Too breathless to do these” the same. |
| 11. Were you short of breath today when | 0. Not at all |
| performing your usual activities outside the | 1. Slightly |
| home such as yard work or errands? | 2. Moderately |
| | 3. Severely |
| | 3. Extremely |
| | 3. Too breathless to do these |
| | Note: |
| | score “Severely,” “Extremely,” and |
| | “Too breathless to do these” the same. |

LRSS was evaluated using the following questionnaire in TABLE 12.

URSS was evaluated using the following questionnaire in TABLE 13.

Baseline E-RS vs baseline LRSS were evaluated and were well-correlated. However, peak change from study baseline in E-RS and LRSS were less well-correlated when measuring changes from baseline at this stage of enrollment.

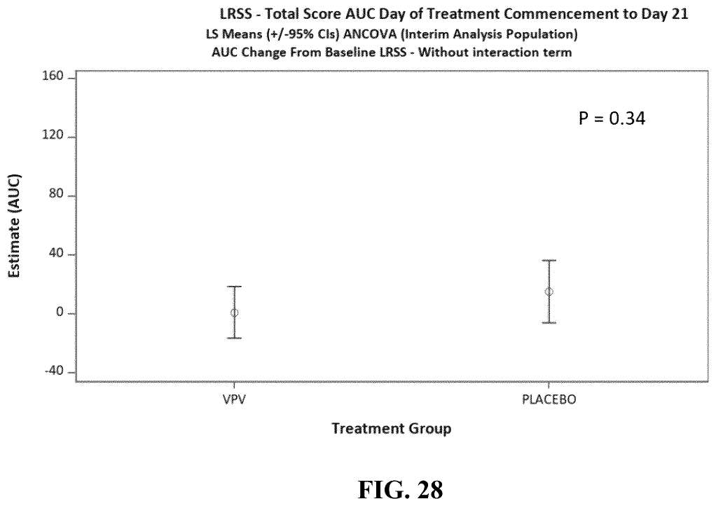

FIG. 28 shows LRSS—Total Score AUC from day of treatment commencement to day 21 for VPV subjects and placebo subjects.

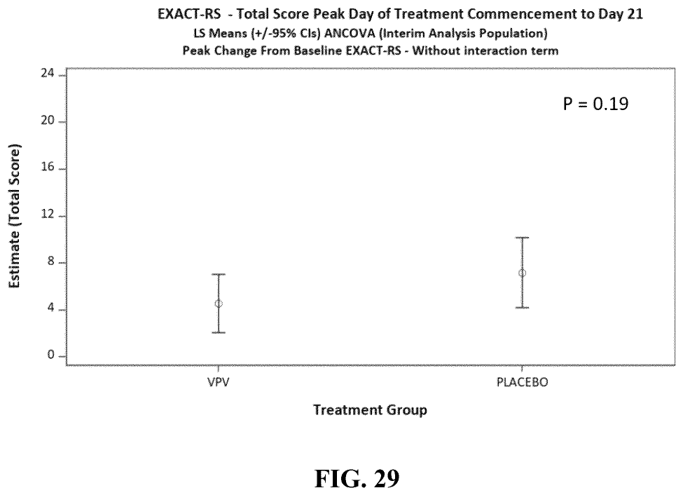

FIG. 29 shows EXACT-RS—Total Score Peak from day of treatment commencement to day 21 for VPV subjects and placebo subjects.

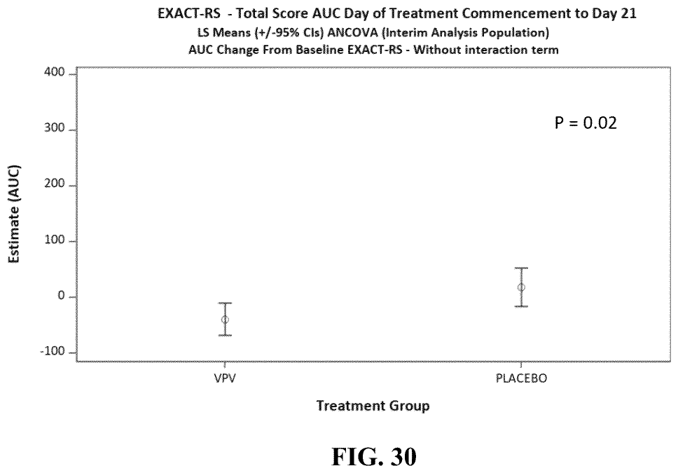

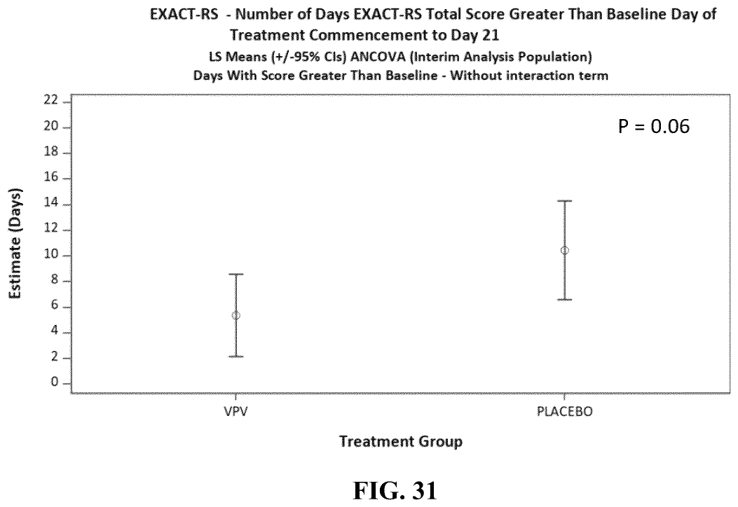

FIG. 30 shows EXACT-RS—Total Score AUC from day of treatment commencement to day 21 for VPV subjects and placebo subjects. FIG. 31 shows EXACT-RS—Number of days that EXACT-RS total score was greater than baseline from day of treatment commencement to day 21 for VPV subjects and placebo subjects.

Baseline E-RS compared to peak change in E-RS for the total efficacy population was found to be impacted by randomization imbalance and a strong effect was not observed at this stage.

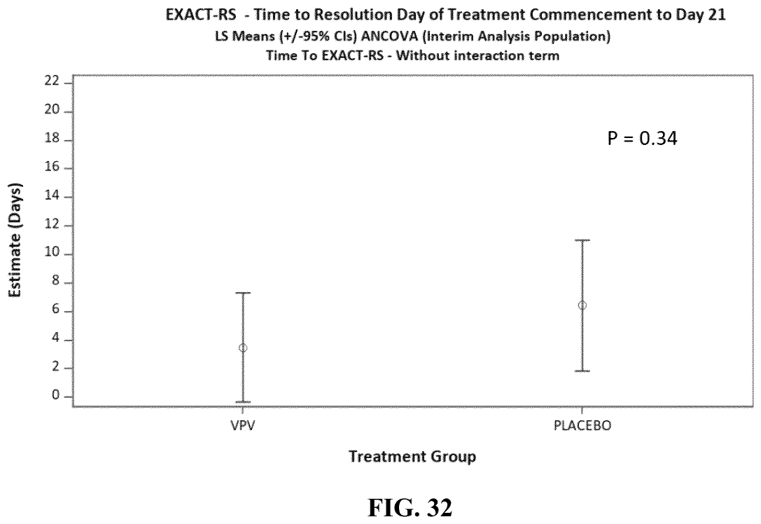

FIG. 32 shows EXACT-RS—Time to resolution from day of treatment commencement to day 21 for VPV subjects and placebo subjects. It was determined that VPV resulted in a quicker time to resolution (less than about 3.5 days) compared to placebo.

TABLE 14 shows the efficacy trend in time to resolution for VPV compared to placebo.

TABLE 14

| | Treatment | | | |
|---|---|---|---|---|
| | Effects and | | Lower | Upper |
| | Difference | LS | 95% | 95% |
| | from Placebo | Mean | CI | CI |
| Time to EXACT- | Placebo | 6.45 | 1.86 | 11.04 |
| RS without | VPV | 3.49 | −0.34 | 7.31 |
| interaction term | VPV − | −2.96 | −9.36 | 3.42 |
| | Placebo | | | |

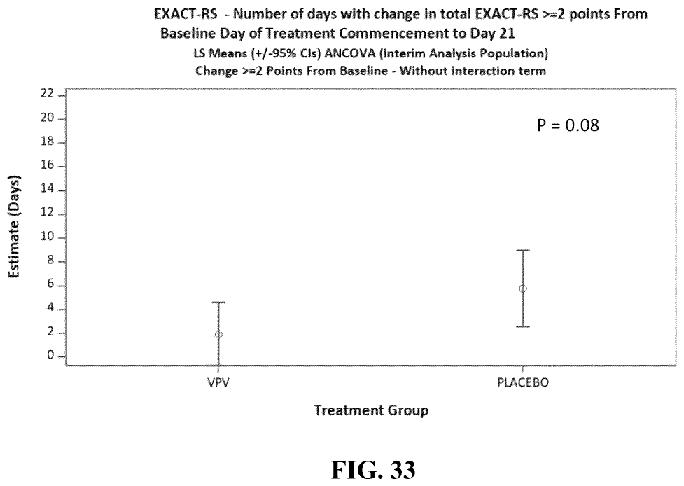

FIG. 33 shows EXACT-RS—Number of days with change in total EXACT-RS>=2 points from baseline from day of treatment commencement to day 21 for VPV subjects and placebo subjects.

TABLE 15 shows the efficacy trend in number of days with clinically significant deterioration for VPV compared to placebo. It was determined that VPV resulted in fewer days with clinically significant deterioration (less than about 2 days) compared to placebo.

TABLE 15

| | Treatment | | | |
|---|---|---|---|---|
| | Effects and | | Lower | Upper |
| | Difference | LS | 95% | 95% |
| | from Placebo | Mean | CI | CI |
| Change >= 2 | Placebo | 5.81 | 2.60 | 9.02 |
| Points from | VPV | 1.96 | −0.71 | 4.63 |
| Baseline without | VPV − | −3.85 | −8.32 | 0.62 |
| interaction term | Placebo | | | |

Upper respiratory tract infections were also investigated. In particular, it was considered whether VPV was effective for colds in COPD patients.

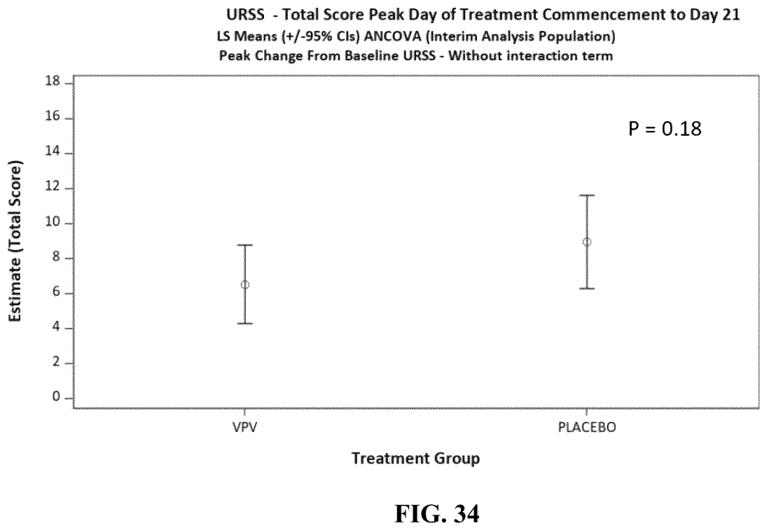

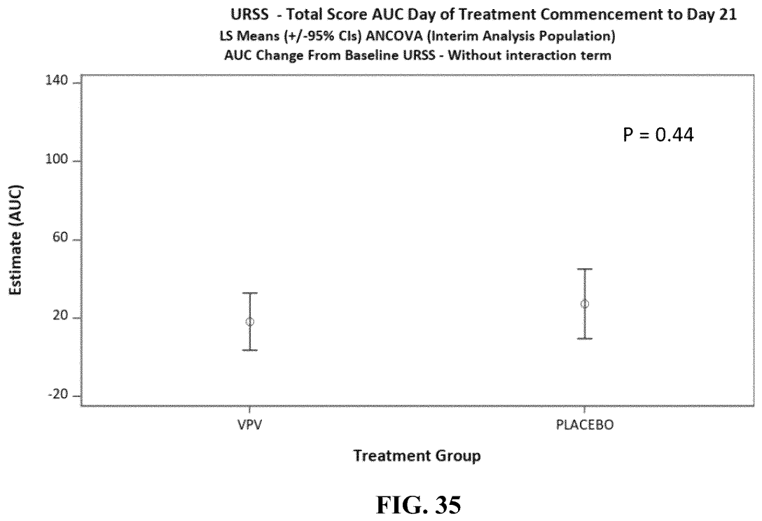

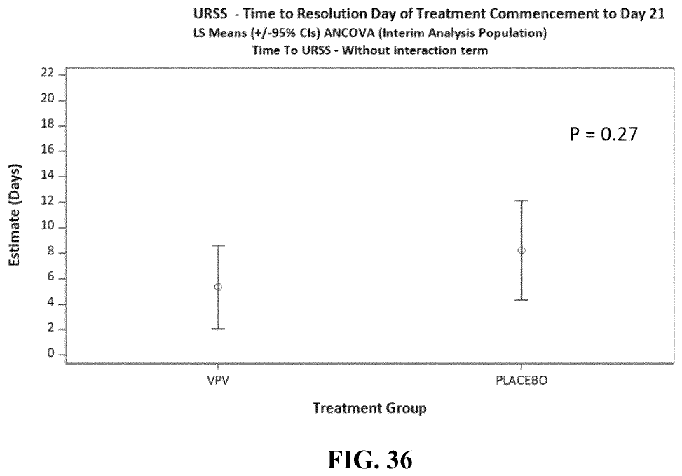

FIG. 34 shows URSS—Total Score Peak from day of treatment commencement to day 21 for VPV subjects and placebo subjects. FIG. 35 shows URSS—Total Score AUC from day of treatment commencement to day 21 for VPV subjects and placebo subjects. FIG. 36 shows URSS—Time to resolution from day of treatment commencement to day 21 for VPV subjects and placebo subjects.

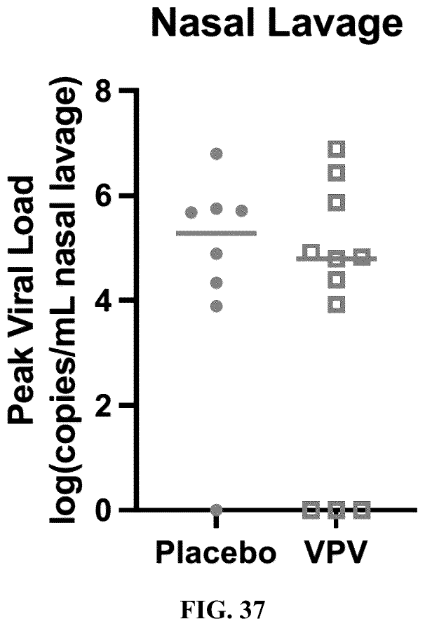

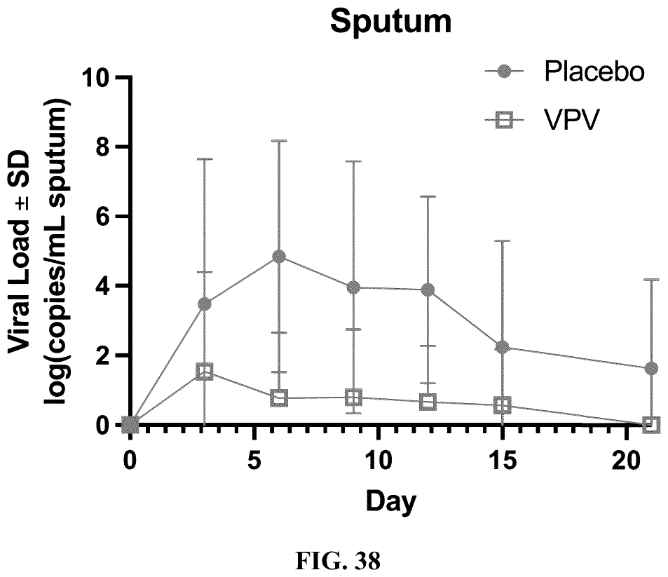

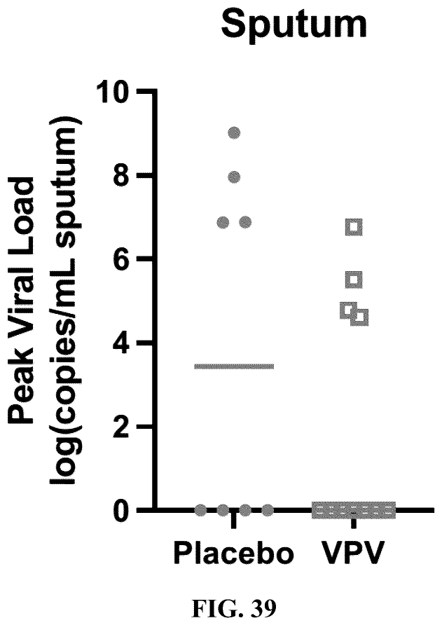

Viral load in nasal lavage was measured, but a significant change between VPV and placebo was not observed at this point in enrollment. It is contemplated that improved sample collection procedures and internal controls may reveal a larger change between VPV and placebo. FIG. 37 shows the peak viral load in nasal lavage for VPV and placebo, demonstrating that VPV lowers viral load and contributes to relief of symptoms. FIG. 38 and FIG. 39 show the viral load and peak viral load, respectively, in sputum for VPV and placebo. As shown in FIG. 38 and FIG. 39, VPV significantly lowers viral load in the lower airway which is predicted to contribute to symptom relief.

### Example 9—Challenge Study

A double-blind, placebo-controlled, randomized study was performed in 40 healthy volunteers challenged intranasally with rhinovirus (HRV 39). Dosing began two days before viral challenge.

An experimental model of RV infection in COPD patients demonstrated a favorable treatment window, with upper respiratory infection (URI) symptoms beginning on day 1-2 with peak symptoms on day 3. Lower airway symptoms and viral load peak were observed on day 9. A potentially broad treatment window (days 2-9) was contemplated, with symptoms being worse in COPD patients (matching viral load).

Parameters evaluated included Upper Respiratory Symptom Score (URSS), Lower Respiratory Symptom Score (LRSS), and EXACT-RS.

Subjects were administered a 1000 mg loading dose of vapendavir free base followed by twice daily maintenance doses of 500 mg each for 7 days. Outcomes relative to baseline scores starting from day of treatment commencement were assessed at 21 days and 42 days.

342 potential subjects were screened in person, with 52 enrolled in the study and baselined. Of these, 44 subjects were randomized and challenged with RV, and 40 were determined to be evaluable subjects.

In comparing vapendavir to placebo, the dosage regime of this study achieved trough blood levels that were substantially greater than protein adjusted EC₉₀. There was no evidence of safety or tolerability issues and strong evidence of antiviral activity. Vapendavir's antiviral activity led to improved symptoms, including a consistent trend towards benefit in time to resolution of both upper and lower airway symptoms and consistent evidence for improvement in upper and lower airway symptoms especially at the 21-day time point.

The final efficacy population demographics are shown in TABLE 16.

TABLE 16

| | Placebo, | | VPV, | |
|---|---|---|---|---|
| | n = 20 | | n = 20 | |
| Male % | 11/20 | (55%) | 13/20 | (65%) |
| Age (SD) | 64.8 | (6.2) | 65.0 | (6.45) |
| Current Cigarette Use | 6/20 | (30%) | 5/20 | (25%) |
| White | 19/20 | (95%) | 17/20 | (85%) |
| FEV1 % (SD) | 60.9 | (10.3) | 63.7 | (12.3) |
| FEV1/FVC (SD) | 0.52 | (0.08) | 0.58 | (0.07) |
| Baseline E-RS | 11.7 | (4.8) | 6.8 | (4.3) |
| Baseline CAT | 20.2 | (8.9) | 15.3 | (7.3) |
| Day of Treatment | 1.9 | (0.9) | 2.4 | (1.14) |

No serious adverse events (SAEs) were observed, nor were there any drug discontinuations due to adverse events (AEs). For the placebo group, there were 34 AEs in 15 participants, compared to 20 AEs in 10 participants in the VPV group. Nasopharyngitis was the only AE that occurred in more than one VPV subject.

TABLE 17 shows the median time to resolution to baseline value of the placebo compared to vapendavir. As shown, vapendavir resulted in a significantly shorter time to resolution.

TABLE 17

| | Time to | Time to |
|---|---|---|
| | Resolution (Days) | Resolution (Days) |
| Score | Placebo | Vapendavir |
| URSS | 21.5 | 8.0 |
| LRSS | 24.0 | 9.5 |
| E-RS | 10 | 5.0 |

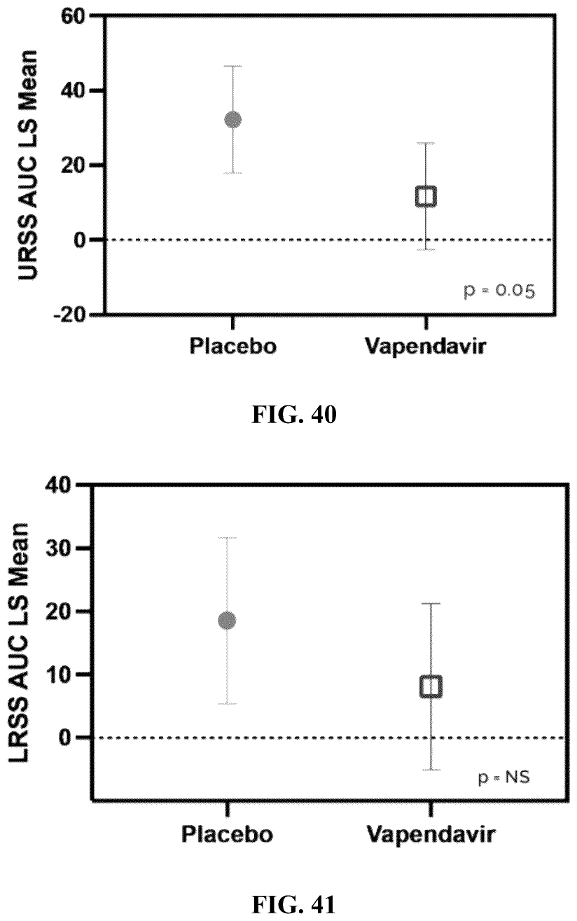

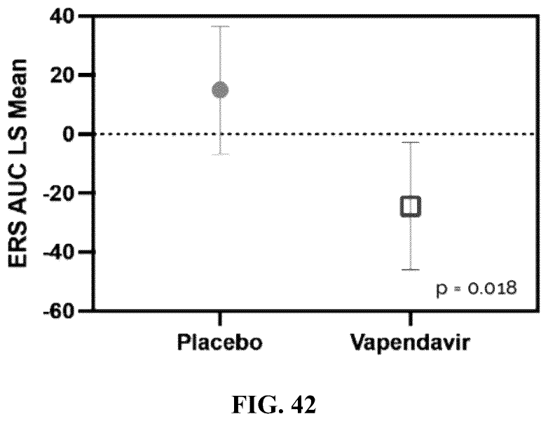

FIG. 40 shows the URSS AUC mean change from baseline (day of treatment commencement to day 21, at 95% confidence intervals) of placebo vs. vapendavir, according to an embodiment of the present disclosure. FIG. 41 shows the LRSS AUC mean change from baseline (day of treatment commencement to day 21, at 95% confidence intervals) of placebo vs. vapendavir, according to an embodiment of the present disclosure. FIG. 42 shows the E-RS AUC mean change from baseline (day of treatment commencement to day 21, at 95% confidence intervals) of placebo vs. vapendavir, according to an embodiment of the present disclosure.

The study aimed to determine if VPV could decrease the severity and duration of symptoms. Trough PK samples were obtained from all subjects pre-dose on Days 2, 3, 4, 5, 6, 7 and 8. Sparse PK samples were obtained after the last dose for Cₘₐₓ, C_{trough}, and elimination phase. Safety assessments of vital signs, physical examinations, and laboratories were collected frequently.

Twenty-one subjects receiving VPV had PK samples available. Mean+SD trough values ranged from 1630 ng/mL+1140 predose on Day 2 to 2460+1130 ng/mL on Day 7. Mean+SD Cₘₐₓ after the final dose was 2600+1190 ng/mL; C_{trough} was 1860+1100 ng/mL; and during the elimination phase the exposure was 1270+720 ng/mL. Overall, the PK results demonstrated good exposures with C_{trough} results that are well above the median protein adjusted EC₉₀ of 678.6 ng/mL for diverse RV serotypes and 845 ng/mL for RV-A16, concentrations necessary to achieve beneficial outcomes of VPV treatment.

Compared to placebo, VPV had a statistically significant reduction in area under the curve (AUC) of EXACT-RS scores (p=0.018), Upper Respiratory Symptom Scores (URSS, p=0.05), and a favorable trend in AUC of Lower Respiratory Symptom Score (LRSS). The median time to resolution of illness also favored VPV for EXACT-RS (10 versus 5 days), LRSS (24 versus 9.5 days) and URSS (20 versus 8 days). VPV treatment was well tolerated, with a favorable adverse event profile.

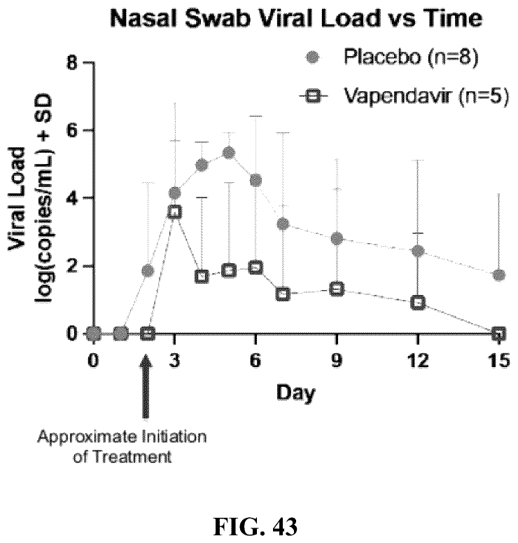

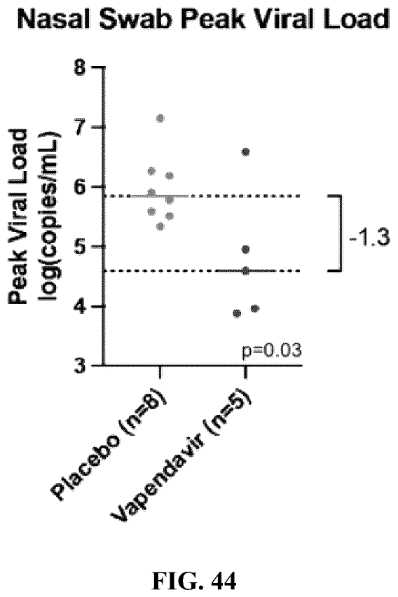

FIG. 43 shows the nasal swab viral load over time with placebo (n=8) vs. vapendavir (n=5), according to an embodiment of the present disclosure. As shown, vapendavir lowered viral load quickly after treatment. FIG. 44 is a scatter plot of nasal swab viral load over time with placebo (n=8) vs. vapendavir (n=5), according to an embodiment of the present disclosure.

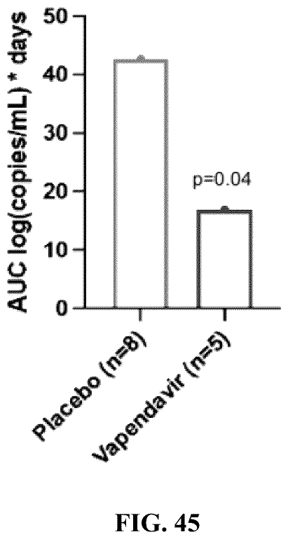

FIG. 45 is a bar graph of AUC virus load (log(copies/mL)*days) of placebo (n=8) compared to vapendavir (n=5), according to an embodiment of the present disclosure.

Compared to placebo, VPV treated subjects had reduced upper and lower respiratory viral load (VL) AUC, peak VL, and time to undetectable VL. VPV shortened the time to undetectable nasal swab VL by 6 days (median 12 days for PBO; p=0.09), reduced peak VL (by 1.3 log copies/mL; p=0.03), and AUC VL (by 25 log copies/mL*days; p=0.04). Trough exposures to VPV exceeded the protein-adjusted EC₉₀ and treatment was well tolerated in this population.

These results demonstrate a consistent beneficial effect of VPV on upper and lower respiratory symptoms, as well as the potent antiviral activity of VPV after symptom onset, in COPD patients challenged with RV and support the advancement of VPV into large scale randomized trials. Treatment of RV may be a transformative therapeutic approach for people living with COPD and other chronic lung conditions.

### Example 10—Phase 2 Clinical Trial Protocol

A multi-center, randomized, double-blind, placebo-controlled clinical trial evaluating the safety and efficacy of oral vapendavir free base compared with placebo in patients with COPD and RV infection will be conducted. Approximately 600 participants will be evaluated to achieve approximately 225 subjects with RV respiratory infections. Two VPV doses will be compared with placebo over a 7-day treatment course. The primary outcome variable will be area under the curve, mean change from baseline of the EXACT-RS (E-RS) from day of treatment commencement to day 28. The secondary outcome variables will be time to resolution of lower and upper airway symptoms, need for medical intervention (steroids, antibiotics, hospitalizations, deaths), viral loads, and pulmonary function. It is contemplated that vapendavir will show improved results over placebo.

### Example 11—Rhinovirus Inhibition in Epithelial Cells

The anti-viral activity of vapendavir free base was evaluated in primary human target cells from asthmatic and COPD donors. People with chronic lung diseases such as asthma and chronic obstructive pulmonary disease (COPD) are especially vulnerable to the effects of rhinovirus infection, which may cause acute exacerbations of asthma, emphysema, or chronic bronchitis in susceptible individuals. Rhinovirus is currently the number one cause of asthma exacerbations and there are potential benefits to treating rhinovirus infection in these individuals. However, when epithelial cells from individuals with asthma were cultured in vitro, deficiencies in the innate immune response to rhinovirus infection were identified compared with cultured epithelial cells from non-asthmatic individuals. Furthermore, epithelial cell genes have been identified from donors with asthma that are differentially expressed compared with those in normal subjects when the cells are exposed to HRV, with gene functions that are related to inflammatory pathways, and regulation of airway repair and the extracellular matrix. Airway epithelial cells from patients with COPD are also different to those from normal subjects, with increased baseline expression of cytokines and increased susceptibility to HRV infection, despite an increased interferon response. Cells from donors with asthma or COPD have previously been cultured in the EpiAirway™ system and have been shown to develop pseudostratified epithelium, with a mucociliary phenotype similar to in vivo airway epithelium. They have also been shown to exhibit barrier properties similar to in vivo tracheal/bronchial epithelium, which includes the development of trans-epithelial electrical resistance, conferred by functional tight junctions. The cultures have been demonstrated to exhibit in-vivo like responses including stimulation of mucus production by TH2 cytokines and secretion of various cytokines and chemokines after stimulation with innate immune challenge agents and TH1/TH2 cytokines. The consequences of differences between epithelial cell responses to rhinovirus infection in individuals with asthma or COPD and those of normal individuals are not fully understood. The antiviral activity of vapendavir free base against HRV 14 was therefore determined in the EpiAirway™ system using cells obtained from human donors with asthma or COPD, and measured using a virus yield reduction assay.

### EpiAirway™ System

The microporous membranes onto which the primary cells are cultured can be found on inserts that are placed inside the wells of cell culture plates. The stratified cells are grown on the membranes at the air-liquid interface (ALI). Cell growth is maintained by addition of assay media to the well. At a specific point in the culturing process, all liquid is removed from the apical (top) surface of the tissue, and the tissues are then fed only through the basolateral (bottom) surface, which remains in contact with MatTek's proprietary assay media. Thus, the tissues are partially exposed to air. Virus can be added to the apical surface of the tissue to mimic exposure of the respiratory tract to HRV via the air. Compounds can be added to assay media that comes in contact with the basolateral surface of the tissue. For this study, cells from two different donors were used; TBE15 cells were cultured from a donor with COPD, while TBE27 cells were cultured from a donor with asthma.

### Cell Lines

The human cervical carcinoma (HeLa Ohio) cell line is a well-characterized host cell line for the propagation of HRV.15HeLa Ohio cells were sourced from the European Collection of Cell Cultures (ECACC, Wiltshire, UK; catalogue number 84121901). Cells were subcultured to generate cell bank stocks in Dulbecco's Modified Eagle's Medium: Nutrient Mix F12 (DMEM-F12; Gibco, Mount Waverly, Australia; catalogue number 11330-032) supplemented with 10% (v/v) fetal bovine serum (FBS; Gibco, Mount Waverly, Australia; catalogue number 16050-122). Cell stocks were frozen at −20° C. for two hours, followed by −80° C. overnight, before transfer to liquid nitrogen for long-term storage.

HeLa Ohio cells were passaged for a maximum of 20 passages, after which a new working cell bank stock was retrieved from liquid nitrogen for further use.

### Viruses

HRV 14 was sourced from the ATCC (Manassas, VA, USA; catalogue number VR-284). Documentation received with the parent stock indicated that prior to receipt, HRV 14 was passaged in the following cell lines: three passages in human kidney cells (HK/3); seven passages in human oral epidermoid carcinoma cells (Kb/7); one passage in human diploid cells (HD/1); ten passages in HeLa cells (HeLa/10) followed by an additional passage in human cervical carcinoma derivative cells (H1-HeLa/1).

Working stocks of HRV 14 were generated by a further three passages in HeLa Ohio cells. Cells were cultured in roller bottles in DMEM-F12 containing 2% (v/v) FBS and 10 mM magnesium chloride (MgCl2; Sigma, Sydney, Australia; catalogue number M-2393) at 33° C. in a humidified 5% CO2 atmosphere (Sanyo MCO-17AIC incubator; Quantum Scientific, Milton, Australia) until more than 80% of the monolayer exhibited cytopathic effects. Virus was then harvested by freeze-thawing and centrifugation at 5000×g for ten minutes (Sigma 3K18C centrifuge; Sigma, Sydney, Australia). The supernatant, containing virus, was aliquoted and frozen at −80° C. for long-term storage.

### Experimental Methods

### Test and Control Article Preparation

Stock solutions of vapendavir free base were made by dissolving the compound in 100% cell culture grade dimethylsulfoxide (DMSO; AppliChem, Darmstadt, Germany, catalogue number A36720100) to a concentration of 4 mg/mL (10.5 mM). The solubility of vapendavir precluded testing of concentrations higher than 4 μg/mL (10.5 mM).

Working solutions were created by diluting the stock solutions in assay media. The assay media used was specific to the host cell. EpiAirway™ serum-free media was used with the EpiAirway™ system. DMEM-F12 supplemented with 2% (v/v) FBS and 10 mM MgCl2 was used for HeLa Ohio cells.

### Antiviral Activity of Vapendavir Free Base in the EpiAirway™ System

All assay conditions were performed in triplicate and experiments were repeated twice. The 96-well plates of the EpiAirway™ system (MatTek Corporation, Ashland, MA; catalogue numbers AIR-196-HTS-D2 TBE15 and AIR-196-HTS-D2 TBE27) were equilibrated according to the manufacturer's protocol. Briefly, 250 μL of the EpiAirway™ serum-free media (MatTek Corporation; catalogue number AIR-100-MM-ASY) was added to a feeder tray that allows the media to come into contact with the basolateral surface of the tissue. The plate was incubated for at least 18 hours at 37° C. in a humidified 5% CO2 atmosphere (Sanyo MCO-17AIC incubator; Quantum Scientific, Milton, Australia). Assay media was then removed from each well.

Nine concentrations of vapendavir free base ranging from 610 ng/mL to 4 mg/mL (1.59 μM to 10.46 mM), were prepared by threefold serial dilution in 100% DMSO. This panel was further diluted in assay media containing a final concentration of 0.04% (v/v) DMSO, to give a range of vapendavir free base from 0.24 ng/mL to 1.6 μg/mL (0.63 nM to 4.18 μM). 250 μL of each dilution was added to the basal surface of the membrane in duplicate wells. The negative control (250 μL assay media) was added to each of the wells designated as negative control wells. Assay media (130 μL) was added to the apical surface of cells to assist transport of compound from the basolateral to the apical compartments, and assay plates were incubated at 37° C. in a humidified 5% CO2 atmosphere for 18 hours. The final concentration of BTA798 in the wells of the assay plate (apart from negative control wells) ranged from 0.16 ng/mL to 1 μg/mL (0.4 nM to 2.61 μM).

The apical surface of the tissue in the EpiAirway™ system was then inoculated with 1.8×105 pfu per well (20 μL) of HRV 14. Assay plates were incubated at 33° C. in a humidified 5% CO2 atmosphere for eight hours, then 125 μL of media was removed from the apical surface of duplicate wells and combined. Samples were stored at −80° C. prior to quantification by virus yield reduction assay.

### Quantification of Virus Titer: Virus Yield Assay

HeLa Ohio cells were seeded in 96 well plates (Corning; catalogue number 3595) in 200 μL of assay media (DMEM-F12 supplemented with 10% FBS) at a concentration of 1.0×104 cells per well and incubated overnight at 37° C. in a humidified 5% CO2 atmosphere. After this incubation period, the cells were approximately 50% confluent.

Of the virus samples harvested from the apical surface of the tissue in each well of the EpiAirway™ system, 10 μL of each was diluted 1:100 in assay media. A volume of 100 μL of each dilution was added to each of three wells in a new assay plate, and then these were serially diluted three-fold across the plate from column 2 to 10, resulting in a total of nine different virus sample concentrations. The remaining wells contained assay media alone (i.e., no virus), serving as controls. Plates were incubated for five days at 33° C. in a humidified 5% CO₂ atmosphere, during which time cytopathic effects (CPE) was allowed to develop.

Virus-induced CPE of the cell monolayer was scored visually and the TCID₅₀ of the virus suspension was determined using the method of Reed-Muench. Resulting TCID₅₀ values for vapendavir free base were expressed as a percentage of the negative control TCID₅₀ value. EC₅₀ values were calculated from the percentage of the negative control value results by non-linear regression analysis. IDBS XLFit4 Excel Add-in Version 4.2.0 (ID Business Solutions Inc., Alameda, CA) was used to perform the calculations.

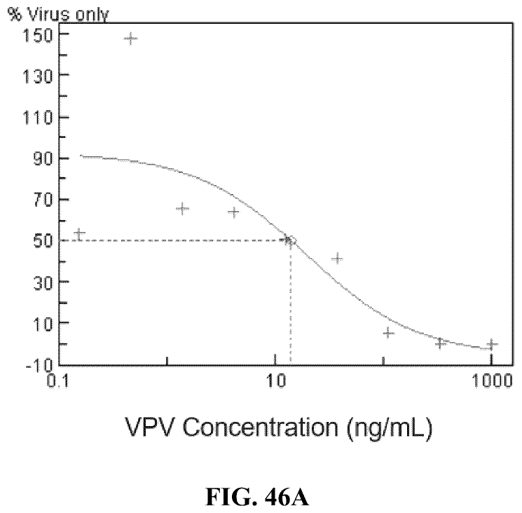

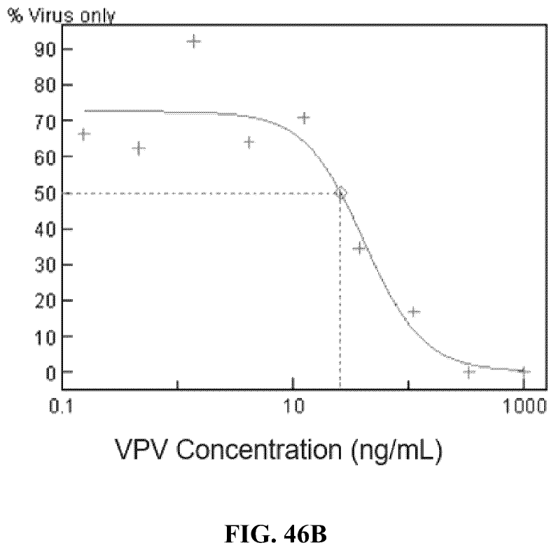

FIG. 46A and FIG. 46B show % virus as a function of vapendavir free base concentration in cells from asthmatic donors, according to an embodiment of the present disclosure. The curves demonstrate a dose-dependent inhibition of virus concentration, as would be expected for a direct acting anti-viral agent. An EC₅₀ value of 13.91 ng/mL is shown in FIG. 46A and an EC₅₀ value of 25.68 ng/mL is shown in FIG. 46B. These EC₅₀ concentrations were exceeded in pharmacokinetic studies in normal humans and in subjects with COPD, indicating that Vapendavir will display potent antiviral activity in humans with asthma.

### Results

Vapendavir free base significantly reduced the titer of infectious HRV 14 in the EpiAirway™ system regardless of the source of donor epithelial cells, suggesting that vapendavir is active in cells with a phenotype that more closely mimics those in vivo. The mean EC₅₀ value in cells from a donor with asthma was 19.80 ng/mL±8.32 (51.78 nM). For cells from a donor with COPD, the mean EC₅₀ value was 33.22±4.51 (86.88 nM). These values are broadly comparable with a mean EC₅₀ of 50.0 ng/mL±22.5 (130.7 nM) in cells from healthy donors. This study demonstrates vapendavir potently inhibits human rhinovirus 14 in cells sourced from donors with asthma or COPD, or ‘normal’ donors, in an in vitro system that closely resembles the epithelial tissue and environment of the human respiratory tract.

This disclosure is not limited to the particular systems, devices and methods described, as these may vary. The terminology used in the description is for the purpose of describing the particular versions or embodiments only and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 compounds refers to groups having 1, 2, 3, 4, or 5 compounds, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

What is claimed is:

1. A pharmaceutical composition comprising a therapeutically effective amount of vapendavir sulfate and one or more pharmaceutically acceptable excipients,
wherein the vapendavir sulfate is in the form of Crystalline Form A,
wherein the vapendavir sulfate has a particle size distribution with a D90 of about 35 μm to about 140 μm, a D50 of about 10 μm to about 25 μm, and a D10 of about 2.5 μm to about 10 μm, and
wherein the pharmaceutical composition is a solid oral dosage form.

2. The pharmaceutical composition of claim 1, wherein the therapeutically effective amount of vapendavir sulfate is about 100 mg to about 1000 mg.

3. The pharmaceutical composition of claim 1, wherein the therapeutically effective amount of vapendavir sulfate is about 250 mg to about 800 mg.

4. The pharmaceutical composition of claim 1, wherein the therapeutically effective amount of vapendavir sulfate is about 500 mg.

5. The pharmaceutical composition of claim 1, wherein the vapendavir sulfate has a polymorphic purity of at least about 90%.

6. The pharmaceutical composition of claim 1, wherein the vapendavir sulfate has a particle size distribution with a D90 of about 100 μm, a D50 of about 25 μm, and a D10 of about 10 μm.

7. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days,
wherein orally administering the loading dose to the subject results in a Cₘₐₓ of about 2400 ng/mL to about 3750 ng/mL of vapendavir; and
wherein the subject is in a fasted state when orally administering the loading dose and the maintenance doses;
wherein the respiratory enterovirus in the subject is treated.

8. The method of claim 7, wherein orally administering the loading dose to the subject results in a Cₘₐₓ of about 3000 ng/mL.

9. The method of claim 7, wherein the subject has COPD.

10. The method of claim 7, wherein the subject has asthma.

11. The method of claim 7, wherein a Tₘₐₓ is about 2 hours.

12. The method of claim 7, wherein the subject is not taking an acid reducer when orally administering the loading dose and the maintenance doses.

13. The method of claim 7, wherein the loading dose is administered within about 1 to about 7 days of the subject displaying symptoms of the respiratory enterovirus.

14. The method of claim 7, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.

15. The method of claim 7, wherein treating the respiratory enterovirus in the subject comprises reducing the duration of viral shedding.

16. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days,
wherein orally administering the loading dose to the subject results in a Cₘₐₓ of about 6400 ng/mL to about 10000 ng/mL of vapendavir; and
wherein the subject is in a fed state when orally administering the loading dose and the maintenance doses;
wherein the respiratory enterovirus in the subject is treated.

17. The method of claim 16, wherein orally administering the loading dose to the subject results in a Cₘₐₓ of about 8000 ng/mL.

18. The method of claim 16, wherein the subject has COPD.

19. The method of claim 16, wherein the subject has asthma.

20. The method of claim 16, wherein a Tₘₐₓ is about 4 hours.

21. The method of claim 16, wherein the subject is not taking an acid reducer when orally administering the loading dose and the maintenance doses.

22. The method of claim 16, wherein the loading dose is administered within about 1 to about 7 days of the subject displaying symptoms of the respiratory enterovirus.

23. The method of claim 16, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.

24. The method of claim 16, wherein treating the respiratory enterovirus in the subject comprises reducing the duration of viral shedding.

25. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days,
wherein orally administering the loading dose to the subject results in a Cₘₐₓ of about 2400 ng/mL to about 3750 ng/mL of vapendavir;
wherein the subject is in a fed state when orally administering the loading dose and the maintenance doses; and
wherein the subject is taking an acid reducer when orally administering the loading dose and the maintenance doses;
wherein the respiratory enterovirus in the subject is treated.

26. The method of claim 25, wherein orally administering the loading dose to the subject results in a Cₘₐₓ of about 3000 ng/mL.

27. The method of claim 25, wherein the subject has COPD.

28. The method of claim 25, wherein the subject has asthma.

29. The method of claim 25, wherein a Tₘₐₓ is about 6 hours.

30. The method of claim 25, wherein the loading dose is administered within about 1 to about 7 days of the subject displaying symptoms of the respiratory enterovirus.

31. The method of claim 25, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.

32. The method of claim 25, wherein treating the respiratory enterovirus in the subject comprises reducing the duration of viral shedding.

33. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg to about 1000 mg of vapendavir free base daily for about 5 days to about 10 days,
wherein orally administering the loading dose to the subject results in a Cₘₐₓ of about 1600 ng/mL to about 2500 ng/mL of vapendavir;
wherein the subject is in a fasted state when orally administering the loading dose and the maintenance doses; and
wherein the subject is taking an acid reducer when orally administering the loading dose and the maintenance doses;
wherein the respiratory enterovirus in the subject is treated.

34. The method of claim 33, wherein orally administering the loading dose to the subject results in a Cₘₐₓ of about 2000 ng/mL.

35. The method of claim 33, wherein the subject has COPD.

36. The method of claim 33, wherein the subject has asthma.

37. The method of claim 33, wherein a Tₘₐₓ is about 2 hours.

38. The method of claim 33, wherein the loading dose is administered within about 1 to about 7 days of the subject displaying symptoms of the respiratory enterovirus.

39. The method of claim 33, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.

40. The method of claim 33, wherein treating the respiratory enterovirus in the subject comprises reducing the duration of viral shedding.

41. A method of treating a respiratory enterovirus in a subject in need thereof, comprising orally administering to the subject a loading dose of vapendavir sulfate in an amount sufficient to provide about 1000 mg of vapendavir free base, followed by orally administering a maintenance dose of vapendavir sulfate in an amount sufficient to provide about 1000 mg of vapendavir free base daily for about 5 days to about 10 days, wherein the subject is in a fed state when administering the loading dose and the maintenance doses, and wherein the respiratory enterovirus in the subject is treated.

42. The method of claim 41, wherein orally administering the loading dose to the subject results in a Cₘₐₓ of about 6400 ng/mL to about 10000 ng/mL of vapendavir.

43. The method of claim 41, wherein orally administering the loading dose to the subject results in a Cₘₐₓ of about 8000 ng/mL of vapendavir.

44. The method of claim 41, wherein a Tₘₐₓ is about 4 hours.

45. The method of claim 41, wherein the subject has COPD.

46. The method of claim 41, wherein the subject has asthma.

47. The method of claim 41, wherein the subject is not taking an acid reducer when orally administering the loading dose and the maintenance doses.

48. The method of claim 41, wherein the loading dose is first administered within about 1 to about 7 days of the subject displaying symptoms of the respiratory enterovirus.

49. The method of claim 41, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.

50. The method of claim 41, wherein treating the respiratory enterovirus in the subject comprises reducing the duration of viral shedding.

51. A method of treating a respiratory enterovirus in a subject in need thereof, comprising orally administering to the subject a loading dose of vapendavir sulfate in an amount sufficient to provide about 1000 mg of vapendavir free base followed by orally administering a maintenance dose of vapendavir sulfate in an amount sufficient to provide about 500 mg of vapendavir free base daily for about 5 to about 10 days, wherein the subject is in a fed state when administering the loading dose and the maintenance doses, and wherein the respiratory enterovirus is treated.

52. The method of claim 51, wherein orally administering the loading dose to the subject results in a Cₘₐₓ of about 6400 ng/mL to about 10000 ng/mL of vapendavir.

53. The method of claim 51, wherein orally administering the loading dose to the subject results in a Cₘₐₓ of about 8000 ng/mL of vapendavir.

54. The method of claim 51, wherein a Tₘₐₓ is about 4 hours.

55. The method of claim 51, wherein the subject has COPD.

56. The method of claim 51, wherein the subject has asthma.

57. The method of claim 51, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.

58. The method of claim 51, wherein treating the respiratory enterovirus in the subject comprises reducing the duration of viral shedding.

59. The method of claim 51, wherein the subject is not taking an acid reducer when orally administering the loading dose and the maintenance doses.

60. The method of claim 51, wherein the loading dose is administered within about 1 to about 7 days of the subject displaying symptoms of the respiratory enterovirus.

61. The method of claim 51, wherein treating the respiratory enterovirus in the subject comprises decreasing a viral load in the sputum, nasal passages, or a combination thereof in the subject compared with a viral load in the sputum, nasal passages, or a combination thereof of the subject prior to treatment.

62. The method of claim 51, wherein treating the respiratory enterovirus in the subject comprises reducing the duration of viral shedding.
